SEQUENCE LISTING (21 sequences)

SEQ ID NO: 1 - DNA, 1653 bp, Homo sapiens
agggacttaggccctgggctgtctccccgagcctctcagtcatgcctgctgtaggggaggagagagtggcctccctctaccctctaaattccttggctgggctccacattaaatttacataaggcagattaacaagagaaaaaccatgtgtaactaagacctatgcatgggaatcccacaaaatacaagactggatgaaggctcagatgaataaagctcatataacatcctacaagggacaggaaacattccttcagaaaggaatagggcttgggtcaggtgtggtggttcacgcctgtaatcccagcactttgggagcctgaggcgggtggatcatgaggtcaggagttcaagaccagcctggtgaacatggcgaaaccccacctctactaaaaatacaaaaattagccaggcacggtggtgtgcacctatagtcccagctactcaggaggctgaggcaggagaattgcttgaacccaggaggctgaggctgtggtgagccaagatcacgccactgcactccagcctgggcaacagaacaagattcctcaaaataaacaaaacaaagaaacaaaacaacaaaaaacaaacaaaaaaacatgaaaatcagctgggtgtcgtggcgtgtgcctgtaatcccagctacttgggaggctgaggtaggagaattgcttgaacccaggatgcagagattgcagtgagccgagatggcaccactgcattccagcctggacaacagagcaagactgtctcaaaaaaaagaaaagaaaaaagaaaaagaaagaaaagaaaagaaaaaaaggaatagaggcttgggatcctggggaggtggtacacaagttaagggagagtgaggggagagagggtaaggtgaatgaagcttgtcttcttatgcggataaaatttctcaggtaagaaaagttagctctgagcagccctccgcctgatactaatactttaccaatggagattttccttttcttttctgtttttgagacagggtctcactttgtttcccaggctggagtgcagtggtgccatcatggatcactgcagcctccatttccctggctcaagccatcctcccacctcagcctcccgagtagctgggactacaaggtgtgcaccaccacgactggctaatttttaatttttttgtagagacgggggtttccctatgttgcccaggctggcttgaattcctgggctcaagtgatcctcccgcctcggcctcccaaattgctgggattgcaggagtgagccacagtgccaggcctggagattttctttatagatagaaatcattttacaaaaggacagcttttcagatctactcctgtgcctgcagtttctaagaataatcccttcaaaatatggcgaagaagtatattttggggtggcacacgctggtctcccayagtcacattttgggtgtgtgtcctgagccccaacactggtcacctcgcaatggcgcttggggtccctgggtgcctagagctgcaggttaccccctcttcttctcctgctctcagcctttctcccgctcctgctgaaattctttcctgcgtgaaggcggagcgcttaaaggcgcctaggacctttatttcgttgttttcctttgcataaaaagaaaacttgttttgtagaagggca

SEQ ID NO: 2 - DNA, 1176 bp, Homo sapiens
acaaacaaaaaaacatgaaaatcagctgggtgtcgtggcgtgtgcctgtaatcccagctacttgggaggctgaggtaggagaattgcttgaacccaggatgcagagattgcagtgagccgagatggcaccactgcattccagcctggacaacagagcaagactgtctcaaaaaaaagaaaagaaaaaagaaaaagaaagaaaagaaaagaaaaaaaggaatagaggcttgggatcctggggaggtggtacacaagttaagggagagtgaggggagagagggtaaggtgaatgaagcttgtcttcttatgcggataaaatttctcaggtaagaaaagttagctctgagcagccctccgcctgatactaatactttaccaatggagattttccttttcttttctgtttttgagacagggtctcactttgtttcccaggctggagtgcagtggtgccatcatggatcactgcagcctccatttccctggctcaagccatcctcccacctcagcctcccgagtagctgggactacaaggtgtgcaccaccacgactggctaatttttaatttttttgtagagacgggggtttccctatgttgcccaggctggcttgaattcctgggctcaagtgatcctcccgcctcggcctcccaaattgctgggattgcaggagtgagccacagtgccaggcctggagattttctttatagatagaaatcattttacaaaaggacagcttttcagatctactcctgtgcctgcagtttctaagaataatcccttcaaaatatggcgaagaagtatattttggggtggcacacgctggtctcccatagtcacattttgggtgtgtgtcctgagccccaacactggtcacctcgcaatggcgcttggggtccctgggtgcctagagctgcaggttaccccctcttcttctcctgctctcagcctttctcccgctcctgctgaaattcttycctgcgtgaaggcggagcgcttaaaggcgcctaggacctttatttcgttgttttcctttgcataaaaagaaaacttgttttgtagaagggcaagcaaggtgcagcttccagaggcacctgggatgatgcccctctagcggtaggcgaactggggagtgtgggcggcgggggaaggcgaggccctggaggacctgttgttt

SEQ ID NO: 3 - DNA, 983 bp, Homo sapiens
ccccaggacatggtatggggctggtggcactgggaggggtttggggagccccattaatcattgaagtttgtaggaggggtggataaggacatgggagaatgattgggtgaggtgctccagggctctctggaagggctgggccccccccttgcatcagccacagtgggggcattgctcttttctatgttggtcatgaaatcyggaagaagtgctttcttatcaatagagaagcataatcagccttgtttttgaaactttggaaaaccatttatggctgggcatggtggttcatgcttgtattcctaatgctttgggaagctaaggaaggaggatcacttgagcccaggagtttgagaccagcctgggcaacatagtaagacccccatctctgcaaaaaataaaaataaattagctgggccttgtgatgcacgcctgtagccccagctatgtggaagactgaggtgggaggattacttgagtccaggagtttaaggctgcagtgagctatggttgcaacactgcactctagcctgcgtgacagagcgagaccctgtctcaaaaaaaaaaaaaaaaaagtcatttatgtccaggtgagggtggggctgggtgaggtggtggttttagggggattgccacgtgggagaaaatgtactgtctttggagtcagctgaactgggttttttcccagccacatttttttttttttttttttttttggtgaggcagagtctccctgtgtcacccaggctggagtgcagtggtgggatcttggctcactgcaacctctgcctccaaggttcaggagattctcctgcctcagcctcccgagtagctgagattacaggcacatgccactatacctggctaatttttgtatttttagtagagacagggtttcgccatattggccaggctggtctcaaactcctgacctcaggtgatctacctgcctcggcctcccaaagtgctgggcctggccacca

SEQ ID NO: 4 - DNA, 1001 bp, Homo sapiens
tgaggcaggagaatggcgtgaacctgggaggcggagcttgcagtgagccgagattgcgccactgcactcccacctgggccacagagcgagactccgtctcaaaaaaaaaaaaaaaaaaaaaaagacaaccctgtttggtaagtactactgtgattcccactttacagatggggaaactgaggcacagaaaggggaagtaacttacctaagtcttacccagccagaaagtggcagaggctgctcctaaccctgcaccatgctgcctccaaacacgggcttgttcccaggaagaagtgctgggttgggggaggggagtccagatcccagggaggaggactcaggaggcctctgggagtcaggcggctatgcaggaggggcttcctggctctttttcacttgtcccctcacccccaccccccgtccccaccttcccaggacttagttggacttccaggcagagttccttgtttgtttttgttttgttttttcctttttttagaracagggtctcgctgtgtcacccagtctggagtgcagtgctgcgatcatagtttactgcaacctcaacctcttgggctcaagcaatcctcctgcctcagcctcctgagcagttaggactactgtgtgcaccgccacaccaagctaatttttttttttttttttgagacgaagtcttgctcttgtcccccaggctggagtgcaatggcacagtctcggcccactgcaacctctacctcccgggttcaagcgattctcctgcctcagcctcccgaatagctgggattgcaggtgcccaccaccatgcccagctaatttttgtatttttactatacatggggtttcaccatgttggccaggctggtcctgaactcctgacctcgtgatccacctgccttggcatcccaaagtgctgggattagaggtgtgagccaccgcacccagccacaccaagctaatttttagattttttgtagatatggggtcttgctaagttgcccagg

SEQ ID NO: 5 - DNA, 501 bp, Homo sapiens
catcttccacctaccccagaaccacgtcacccagtggttcctaacctgtctcagattttaaatccctttgaaaacctgcagaaaagcatggactctctccagaaaaaggctctttcatccacagactctgttggtgtgcagggaaggactgtttaggtccaacctctagcttctctgtcgtatgagattctgactctgaaaccaacctcaaaggggcaaatcattctgaccctgtgacacctctgtaagasacacttgccagcaacctattcaaagtctgtcttgaacctgtttctattttctttcttgaccacctcttaaagaagacattgcacaagtttgcccctgccatttaaatcctttgtcctaaatttacagcccctttctggggctttcagcaaggatcgactttcctgcgtctttgggtgactctccactgtatcatcggctcatttctggaagaggaccatcctttggggcatgtgtaaggccccttttgtg

SEQ ID NO: 6 - DNA, 2120 bp, Homo sapiens
tctcctcctcctcttcttcctcgtcatgttttagcataagaagctttattgagctagaattcacataccatacaattcatttaatgtgcacaatgcaatggttttttgttgctgttgttgttattgttttcgtttttatttttgagatggagtttcactcctgtcacacaggctgaagtgcagtggcgtgatctcggctcattgcaacctctgcctcccgggttcaagtgatccttctgtttcagcctcccgagtagctgggattacaggcgcccaccaccacaccgggctactttatgtatttttagtagagatggggtttcaccatgttggccagcctggtctcgaactcttaacctcaggtgatccacccgcattggcatcccaaagtgctaggattacaggcatgagccatcatgcctggcctaatggtttttatttttatttttaaattttttgtagagatggagtcttgctatgttgcctaggctggtttcaaactcttggtgtcaagtgatcctcccacctcggcctcccaaagtgctaggattacaggtgtcagtcaccaaaccccgcctcagtggtttttagtatattcacagttatgcaaccatcagcatcataaattttaagctgtgcatggtggctcatgtctgtaatccaagcactttgggaggctgaggctgaactcacttgagcctagaagttcaagaccagcctgggcaatatagtgagaccctgtctctattaaaaaaaaaaaattaaaacattttcatcactccaaaaagagatttcacaccattagcagttactctctacttttcttccatctctcagcctctggcaaccactaatctgctttctgtctctatggatttgcctgttctggacatttcatataaatgggatgatacactatgtggtcttttggggttgagtttgcaccttagcatattgcctttagaaacctgtctgcctcataccattacagaggagctgttgtttgtactgaccatttagtccatcacaggtattgatggagcctctaagcatggaatagtgctggccccacagcaggaacgcggcagccatgggtggcgtattagttactggttgctgtttaacaaatcacgccaacacttggcagactgaacagcaagcatttatgatcccgtagtttctgtcaatcaggaatacaggagccgcttcgttgggtggttccggtccagggtctcatgaggctgccattgagctttcagtggaggctgcaggtgtctggggctggaggtacctgccacagcgtggttggcaggctcagtgactggctggctgttggccagatatctcagttcctcttcatgtgggcttcttcatagggtggcttgagtgtccttatagcatggccactggcttaccccagagcaagagatgagagcrctggagagagacagagagagagagagaagaggggaggggaggggagggggtgtataagcccaagactgaagccccagtgatgtttcataacctaattttttcattaattaattaattaattttagagatagggtctctgctgcccaggctgaggtgcagtggcacaatcagagctcactgcagcctccagctcctgggctcaagcaaccctccagcctcaccctctcaagcagctgggactacaggtatgcatcaccatgcctggctgctgctgctgctttttttgtttgtttgtttgagacggagtctcactctgtcacccaggctggagtgcagtggcgcaatctcggctcactgcaacctccccttcccgggttaaagcgattctcctgcctcagcctcccgagtagctgggactacaggcgtgcgccactagtttctgtatttttagtagagatggtttcaccatgttagccaggctggcttgaactcctgacctcaggcaatctgctcgcctccccgcctcgggctccaaaagtgctgggattacaggtgtgagctaccaaacctggcccggattcttttataatctaatgttaaaggtgctatcccatcactgtgccatattctttaggtcacattgactgtgaatgtgggtgggggctacacaacggtgagaatct

SEQ ID NO: 7 - DNA, 601 bp, Homo sapiens
ttaattggtcagcaatggaagggatttcttccttagcctttggtgctgacctcctgtttctctggtggcagtgccttgcgaagtgcttgccactgtcccctggattttgagggaagggagcatccgagcccgcatgcctgggctcccgcagcagggtccccggagcatctttgcagggatagaggccgcagggaggactccagcttggtccttgggagatgtgttcctgcaacccatgccttttccagcgggttcctagggctctgctgtttgactcatggagaagccggtggttttggtkttgtgctaaatcgaagcccacctagtgggctctgggccgaaggctcccagctgtggaggtagctatgcccagggcctggggttgggagaaggggttgggtctttagactgggctggagctctagtgtcttttttccctctttggggttctgttatttcctggagcggctgaggtgggggaagaagcaccacagatgcaaggagtgggcggcccctcccctcacactcttcctttttatctccagcccaatagggcatgaattttcctttagacctgggccttgaaaacagatccacatgg

SEQ ID NO: 8 - DNA, 1001 bp, Homo sapiens
cactttgggaggctgaggcaggaggatcgcttaagcccaggagttcgagaccatcctgggcaacgtagggaaatcctctctctacataaaataaaaaacaagccaggtattgtggcacatgcttgtgatcccagctacttgggaggctgaggtgggaggattgcttgagcctgagaggttgaggctgcagtgagccatgatcataccagtgcactccagcctgggtgacagagtgagaccctgtctcaaaaacaaaaaagagagagagagagagagacagagagagacttctagtgtgaaagacagagacccaaatgaacagaaaaaaaaatttgagaagtcacaaacagggtcgtatggggaaaaaaaggcacattcataaaataataagctgttaggtattaagatagcagaagtaaaaatagaagaagtaaaaatttcaacagaagagttggaagagtgaaaaacaaatttccagaaagtagaacacaaagacttaaragatggaaaataggaaagaaaatatagaaaaatttgacttcagtccaggagacccagcatgcaccacataggagctcaagaaaaaaaggcaagagaaacggaggaaggaaaagtatgtaggaaacaattctagaactgaaggcatttctacttttaaacagtctcgaagtagtacccaataccatgaataataaaaggcccacgctataggacatcatcatgaactttcaaactccagagatagagagaagttactaaatgtcatcagagaaaaaaaggccacatacagatcagagaatgagatggctttagacttttcaaaagcaacagtagaaactacaagacaggccaggcgcaatggctcacacctgtaatcccggcactttgggaggccgaggcgggcagatcactaggtcaggagttcaagaccagcctggccaacatggtgaaaccctgtctctactaaaaatacaaaaattagctgggcgtggtggcatgtg

SEQ ID NO: 9 - DNA, 1492 bp, Homo sapiens
catccggtcctcagaccttggtgctcagagagagagagagagagagagacacagatagacagacagacagacagacagatggacaggtggacagacggacagacagatggacagatgcactgcttttcagacttgggatcctcagatgagaattttaaaagataaataatggtttgtttgtttgtttgtttgttttgagacggagtttcgctcttattgcccaggctggtgtgcaatggcgcaatctcggctcactgcaacctccaactcccgggttcaagagattcttccgcctcagcctcctgagtagctgggattacaggcatgcaccatcacgcccaactaattttgtatttttagtagagacggggtttctccatgttggtcaggctggtctcaaactcccgacctcaggtgatccacctgcctcggcctcccaaagtgctgagattccaggcgtgagccactgcatccagccgataatggtttttttggtttttgttttttgagacaatctcgctctgtcacctaggctggagtgcagtggcatgatctcaggtgactacaacctctgcctcccaggctcaagcagttctcgtgcctcagcctcctgagtagctgggactataggcgtgcaccaccacacctggctaatttttgtgttttttagtagagatggggttatgccatgttggcctggctggtcttgaactcctgagctcaagtgatccacccgcttcggcctcttaaagtgttggaattacaggtgtgagtcaccgcgcccgacccaaaagataaatcacgtccatgagttgttgaaagacttgtggttgcgtccaattattccaatttcagctttccccatttcacagtgtatgtttccctctactcagttatccrattattcatgactagatgagattaggggctgcctcccacggtgggctttcatcctattcatgactgtgtcatcatttttttttttgagatggggtctcactctgttgcccagcctggagtgcagtagtgcgatcacagctcactgcagcctcaatctccccaggcttaggtgagcctcttacctcagcctcctgagtagctgggactacaggcacgtgccatcaagcccaactaatttttcgtgtctgtattttttgtggagatggggtttcaccatgttgcccaggctagaatgtgcctttcatttttgaaaccacactccatccatggctgcctaagtggtttacaggtcttttgtctttaaagctgttgccatagtaaacaacatctctttttctttttttttttgagatggagtcccgctctgtcgcccaggctggagtgcagtggcgccatctcggctcgctgcaagctccacctcccaggttcacaccattctcctgcctcagcctcccgagtagctgggactacaggcgcccaccaccacgcccggctaattttttgtaaacagcatctttggaaagagattttg

SEQ ID NO: 10 - DNA, 801 bp, Homo sapiens
cactttgggaggccaaggtggtcggcttacaaggtcaggagtttgagaccagcctggccaatatggtgaaaccctgtctctactgaaaatacaaaaaaattagccgggcgtggaggcacatgcctgtaatctcagctacttgggaggctgaggcaggataattgcttgaaccaaggaggcagaggttgcaatgagccgagatcgtgccactgcactccagcctgggtgacagagcaagactccgtctcaaaaaaaaaaaaaagcctgagtaggggtgaggtgggaacaggagggtgcagcaggtgtcagctggcttcaccactggagccctcagaggaaagaggaaaaagcggctcctctccttccccaagcagggtctcccaggggtctggttgggagaygttagctgggctctgggtcttctcgggtggggtgcctgggggagggcagggaagctgggtctggaggcccctgagtggctgtcctcacctgccctgtccttctgcaggactgccccattgcctgggccaacctcatgctgtttgactacaaggaccagcttaagaccggggaacgctgcctctacatgtggccctccgtcccaggtcggcccaggcccaggagggagaggcgttgggagtgtgagggtcccagagatgctggtcacccctctacaacttcatctgcccctgtgttcagatgagaagggcgagctgctgaaccccacgggcactgtgcgcagtaaccccaacacggatagcgccgctgccctgctcatctgcctgcccgaggtggccccgc

SEQ ID NO: 11 - DNA, 801 bp, Homo sapiens
gccctctcccacctgcagtccccactcgaccccagcaccctgctggctgaagtctggtgagcccaagccccgccacaagggttcctcccacccctgggaggccggtagaggagcccctgctgactgcccgctctctggcctggcagcgtggagcagtgcaccttcatggactccaagatgaagcccctgtggatcatgtacagcaacgaggaggcaggcagcggcggcagcgtgggcatcatctttaagaacggggatggtgagggcctggcctccccacaccccgcctgtactgccctggggggtcctggggtgctcctagagtgggggtggagaagacagaatcctgggacttaagggcttgggtgtagctggaagcagagaacctaccagaaactcaygcttctcctcccaccggccggtggcacagacctccggcaggacatgctgaccctgcagatgatccagctcatggacgtcctgtggaagcaggaggggctggacctgaggtgaggacccccaccccacatcgtcccttggtgtctgtgcccagcctgggagtctgtgcccctggaggggtccttgttgaaggtggcatgaccatctcagccggggaaagggctttcctaggaagacccggaggcggtttaactctaggccaggaggcggcgggcagcaggatgcgtgaaggctgctcctgaggcttagtgtgtaccctgctctgttgctttagttgcccaggtgcccctggcttccccagcccatcttgggaagcagtggcgtcttctcccaaggcctctg

SEQ ID NO: 12 - DNA, 801 bp, Homo sapiens
tagtcccagctacttgggaggctgaggcagaagaatcacttgaacctgggaggcggaggttgcagtgagccgagatcatgcccctgcactccagcctgggcgacagagtgagactgtctcaaaaaaatgtatatatacagatacatatgtattttttttccttcttgtgtcctctccatgtgagaaggtgggatggggcggtgaggctgctggggccacacatgcttgccaggaccttccctctggtgaccagtccctgcaaaagcagctgctcccatgctcctcccagagccatttctccagtaggggaggcgaggtcacttgggaactgggggctctggggccaagatgtctttggcaccttcatttgagggtgggagcggaatagagagcttttcctsagatgctgggagctctctactaaccatttcattcagtgactctgaagtccccagagagggacgcatcccagagcaaggtccgggcccccttaacgtggacaccgctgtgatttgtttgcaggactccctggcactggggaaaacagaggaggaggcactgaagcacttccgagtgaagtttaacgaagccctccgtgagagctggaaaaccaaagtgaactggctggcccacaacgtgtccaaagacaacaggcagtagtggctcctcccagccctgggcccaagaggaggcggctgcgggtcgtggggaccaagcacattggtcctaaaggggctgaagagcctgaactgcacctaacgggaaagaaccgacatggctgccttttgtttacactggtta

SEQ ID NO: 13 - DNA, 801 bp, Homo sapiens
ctcccgccagactgcctgggtcctggcgcctggcggtcacctggtgcctactgtccgacaggatgccttgatcctcgtgcgacccaccctgtgtatcctccctagactgagttctggcagctccccgaggcagccggggtaccctctagattcagggatgcttgctctccacttttcaagtgggtcttgggtacgagaattccctcatctttctctactgtaaagtgattttgtttgcaggtaagaaaataatagatgactcaccacacctctacggctggggagatcaggcccagccccataaaggagaatctacgctggtcctcaggacgtgttaaagagatctgggcctcatgtagctcaccccggtcacgcatgaaggcaaaagcaggtcagaagckaatactctgccattatctcaaaaatcttttttttttttttgagatggggtcttcctctgttgcccaggctggagtgcagtggtgcaatcttggctcactgtaacctccgcctcccaggttcaagtgattcttctgcctcagcctcctgagtagctgggattacaggtgtgcaccaccgtacccagctaatttttgtattttagtagagacgggggtttcaccatgttggctgggctggtctcgaactcctgacctcaggtgatccacccgcctgagcctcccaaagtgctgggattacaggcatgagccaccgcgcccggcccactctgccattgtctaagccacctctgaaagcaggttttaacaaaaggatgaggccagaactcttccagaaccatca

SEQ ID NO: 14 - DNA, 401 bp, Homo sapiens
cccaggttcaagtgattcttctgcctcagcctcctgagtagctgggattacaggtgtgcaccaccgtacccagctaatttttgtattttagtagagacgggggtttcaccatgttggctgggctggtctcgaactcctgacctcaggtgatccacccgcctgagcctcccaaagtgctgggattacaggcatgagccaccrcgcccggcccactctgccattgtctaagccacctctgaaagcaggttttaacaaaaggatgaggccagaactcttccagaaccatcacctttgggaacctgctgtgagagtgctgaggtaccagaagtgtgagaacgagggggcgtgctgggatctttctctctgactatacttagtttgaaatggtgcaggcttagtct

SEQ ID NO: 15 - DNA, 601 bp, Homo sapiens
ttaactttcactgagacctttgcgtgagtgaacaaatcttgttaaatttcatgccctgaacttcatctgtatgtaggattatatttttgaatttcccccgagaataatcattgtaaccttttatcaagagtgggtttgttctcagcagaggcatcaacccacccccgtaacccactcaccgcagggaataaaacaaccattgtcctatggagaagccggcaacgtgaacaccagaggactgcacctgtttgttcactgttatcaaatgccaggctgggggctacccctggtaagagggctyggacttcttaaaccacagagcaacataaactctcctgtagaaatcttttgtgtgcaaacagaagtttagcagcatgctttcttcctgggaggaagaaagagcacgccttttaccatcgggccttcaagatacattcattctttgcaaactgtctgttatgaatactaagcttcccatgagtgtccagcttcagtttaaaaggaagcaatgaaagactcatgaaatgaaaacgatatgaatatagcatcaaaactatgtctgtcttgtgaagaagtaattacttgagtcacttatagaaag

SEQ ID NO: 16 - DNA, 801 bp, Homo sapiens
aaatcaaaactgtataaattagatatattttctaaaaataaggtgattgtgcagaaaactaaagattcatggaaatagatataacagcttggatattactaagcgctaatgatagttatctcattattgcaaggatttgatgatatggtccactgttcacctgaaaccaccaaacacagcatgtttcatatctcagttttcagtcacagagcatttccagagtttactcctgttcagctgagatacagtattatggtatccttcacactaaacttacaaaatctataaagaaaagcaagggcatcacagggcatcattgcttataaaacaaataaatcactgatatttaaatgccttagagtgttcctcaatgtaacaaatatgacagtaaccctacatayacaattgccttatattgataatgaaagcatatttgccattttggatatattccttacctgtcaagttctttaatccaagttcttgaagtccaaagtttccatcttttctgtagtttaaaaatattgccaaggcatatcgatcctcataaagttttgtcccacgaataatgcgtaaattctccagaggcaggtaacgaaactgattaagagccactaacacgtagcctgtgacttctcgaacagactgaaaagacacaaacagttgcctgtgttataaaacgaatttgtcactctgtatatgtagcaatttagattaaaatgagttattaaactctaattttcagtttttagtttaatacattattttccatatcatattctaaagaattggcttcataat

SEQ ID NO: 17 - DNA, 5273 bp, Homo sapiens
atttcaacagtgattgaatatttatattctgcatgattaaaatattatttagatgagcaaacattaaactactatggatatttttctatgatgagaaaaatttcttttcttcttttttgagacagtgtttcactttgtcacccatgtgggactgcagtggaacaatcttagaccttggctcactgcagcctctgcctccagggctccagagatgctcccacctcagcctcccaaagtatatggagtagaggtgtgagccattgctcctggccaataaagatttcttacagtagagttaaaactggaaggagtcttggagatgctgtggactccaagacttcctccactttagagatgaaagactaagaaggtttggataatttacccaggttatatggctaattgacacaatttgaaccacaactgagggttttaggaaggagctcagggttattccaccatgctcttttcttttattcttccatgtcacctcctcccttcttccatccttcccttctttctttcttgcttcctttacatgtgcatggagtttttaccrttactctaaaactatcttaagcactggaattatgaagataaagagacactgacaataaaagctcaaattttatgatttaaaaaaaaagcttttaagttcaggagtacaagtgcaggttgttacataggtaaacttttgtcatggaagtttgttgtacagattatttcatcattcaggtattaatcctagtactcactggttgtttttcctgattctctccctcctcccaccgtccaccctctgaaaggctccagtgtgtgttgttcgcctctctgtgtccatgtgttctcattgttcacctctcacttatgagtgagaacatgtggtgtttggttttctgttcttgtgttagtttgctgaggatgatagcttccagcttaattcatgtccctgtaaaggacatgactttattcttttttatggctatatagtttccatggtgtacatgtcccacattttttttatccagtctattattgatgggcatttaggttgaatccatgtctttgctattgtgcataatgctgtaatgaacatacacatgcatgtgctattatagtagaatgatttatattcctttgggtatatacccagtcatgggattgctgggttgaatggtatttctgtttttaggtcttcgaggaattgccacactgtcttccacaatggctgaactggtttacattcacaccaacagtgtataagtattcttttttctccacaacctcaccagcatctgttatttttttgactttttaataatagccattatgactggtatgagatgatatatcattgtggttttagtttttaatttcccggctgggcacggtggctcatgcctttaatcccagcactttgggaggctgaggcaagtggatcatgaagtcaggagattgagaccatcctgactaacatggtgaaaccccgtctctactaaaaatacaaaaaattagccaggtgtggtggcgggcacctgtagtcccagctactcgggaggctgaggcaggagaatggcatgaacctgggaggcggagcttgcagtgagctgagatcccgccactgcactccagcctgggtggaagagcgagactccatttcaaaaaaaaaaaaaggaatttctctagtaattagtgatggttagcttttttcatatgattgttggccacacatatgtcttcttttgaaaagtgtctgttcatgtcttttgcctacttttttttgtggggtcgtttgttttctttttcttgtcaatttgcttatgttccttatagatactggacattagacctttgtcagatatagagtttgcaaaattttctcccattttttaggttgtttctcccattttgtaggtttactctgttgatagtttcttttgctgtgcagaaactctttagtttaattatatcccatttagcaatgttttgctttagttgcaattgctgtgggtgccttcatcatgaaatctttgcctgtgtctgtgtcctaaatggtattgcctaggttgtcttccagggttttcatagtttcaagttttacatttaaatctttagtccatcttgaataaatttgtgtatgtggcataaggaatgggttcagtttcagtcttctacatatggctagccagttatcccactaccatttattgaatcaggagtcatttctccattgcttgtttctgtcacctttgttgaatatcagatagttgcaggagtgtggtcttttttctgggttctctattctgttctctatttgtctatgtgtctgctttttcaccggtaccttgctgtgttggttactgtagccttgtactatactttgaagtcaggtagcatgatgcctccagctttgttctttttgcttaggattgccttggctatgcaggtcttatttggttccatatgcattttaaaatagttttctctaattctgtgaagaatgtcaatggtagttgaataggaatagcattgagtctataaattgctttgggcagtatgaccattttaatgatattgattcttcctatccatgagcatggaatgtttttctatttgtttgtgtcatctctgacttctttgagcaggggtttgtagttttccttgtagatctcttccacctacctagtcagctgtattcataggtattttattctttatgtggcattatagtttcttccttgattttgttcttagcttgactgttgttggtgtataggaatgctaataattttttcacattgattttatatcctgagactttgctgaagttgtcagcttaagaagcctttgggctgagactatggggttttctagatatagaatcatgttatccacaaacaggggtggtttgacttcctctctttctatttgtttatttatttctcttgcctgattgccctggccagaactttcgatattgtattaaataggaggagtgagagagggtatccttgtcttgtgacaatttcaaggggaatgcttccagctattgcttctattgagatattaataatcatgtaatttttgcctttagttctgtttatgtgatgaatcacatttatttacttgcatatatttaaccaaccttgtttcctggaaatgaagcctacttgatcataatggataagctttttgatgttctgctagatttagtttgctagtatttcattgaggatttttgaatgaacatcaaaaatattgacctgaagttttcttttttgcgtgtgtctctgccagattttggtatcaggatgatactggtctcatagaatgagttagggaggaggtccttctcaattttttggaatagtttcagtaggaatggtaccagctcttctttatacctctagtagaattcagctgtgaatccatctggtcctgggcttgttttgcttgtaggatatttattactgcctcaatttcagaactcattattggtctgttcatggattcagtttcttcctggttcagttttgggagagtccaggaattcatccatttctctagattttctagtttatgagcatagagaaaggccacatcaactaccaagggaagccagtcagactaactgcagagctctcagctgaaattctctatgccagaagagattaggagccaacattcagcattcttaaataaaagaaattataacccagaatttcatatatgaccaaactaagcttcataagcaaaagagaaataagatccttttcagacaagcaaatgctgagggaatttgttaccaccagacctgccttgcaagagctcctcaaggaagcactgaatatgaaaaggaaaggttgttagtagccactacagaaacacactgaagtacacagaccagtgatgctgtaaagcaaccacataaacaagtctgcacaataaccagctaaacatgatgacaagagcaaatccacacatatcaatagtaaccttaaatgtaaataggctaaatgccccaattaaatgacacagaatggcaatctggataaggaaccaagacccattggtatgctgcctctaagagacccatctcacatgctatgacacacataggctcaaaataaagggatggaagaaaatcttccaggcaaatggaaaacagaaaaaaggcaggggttgcaattctaatttttaccaaaacagacttcaaaccaacaaagatcaaaaaaagacaaagaaggtcattacatagtggtaaaaaattcaattcagcaagaagatctaactattgtaaatctatatacttcccacacaggagcacctagattcataaagcaagtttagagacctttgaagagacttacattcccacacaataatagtgggagactttaacacctcactgacaatattagatcatcaagacagaaaattaagatattcaggacctgaacttagcacttaattgaatgggcctgatagatatctatagaactctccaccacaaaacaacagaatatacattcttctcattgccacatggcaattactctaaaattgattacacaatcaaaagtaaaacactcctcagtaaatgcaaaagaactgaaatcatgacagtctcttgtaccacagcacaatcaaatttgaaatttagactaagaaattcactcaaaattatacaattatgtggaaattgaataacctgctcctgaatgacatgataaacaataaaattatgcagaattcaagaagtttttgaaactaatgagaacaaagataccatgtcccacaatgtctgggacacagctaaggaagtgttaagagggaaatttgtagcactaaatgaccacatcaaaaagttagaaagatctgaagttattataacaacataatatcacaactaaaagaattagagaaccaagagcaaagaaatgccaaagctagcagaagacaagaaataaccagcatcagaactgaactggaggagagagacatgaaaaacattcataaaatcaatgaatccaggagctgtttttttgaaaaaaattaataaatagaccactagctagactaataaagagaaaaagagaaaattcaaataaacacagtaaaaaatgacaatgggaatattaccactgaccccacagaaatacctattttatggttttttaaaatgcaaataaaaaatttgaaacttatccagagagaagtctaattaagcaatgcccttaaactgtgcataaagagcacccttcccagcctta

SEQ ID NO: 18 - DNA, 610 bp, Homo sapiens
ttgcccagggtagagagcaatggtgcgatctcaataagcagctcattttgattacaggtatacatgaagtaaaattcatgaagtaaaattcattataccaaaaagcctcccacagaactttcatgcaccctgagctatgtgaactgaaaagtaacagtgggatagcaagaaccagcttacaaaataaattcctatcgagtgtttttcacagaaggatgataattttaggttgcttacatcatatatttccaatcttaatgagaaaaacctactctgatgatctgatgttattgaagtattctgagaaagaattgctgatgggatttagtgagctactgaataagagaaatcaaaactgtataaattagatatattttctaaaaataaggtgattgtgcagaaaactaaagattcatggaaatagatataacagcttggatattactaagckctaatgatagttatctcattattgcaaggatttgatgatatggtccactgttcacctgaaaccaccaaacacagcatgtttcatatctcagttttcagtcacagagcatttccagagtttactcctgttcagctgagatacagtattatggtatccttc

SEQ ID NO: 19 - DNA, 5363 bp, Homo sapiens
atttcaacagtgattgaatatttatattctgcatgattaaaatattatttagatgagcaaacattaaactactatggatatttttctatgatgagaaaaatttcttttcttcttttttgagacagtgtttcactttgtcacccatgtgggactgcagtggaacaatcttagaccttggctcactgcagcctctgcctccagggctccagagatgctcccacctcagcctcccaaagtatatggagtagaggtgtgagccattgctcctggccaataaagatttcttacagtagagttaaaactggaaggagtcttggagatgctgtggactccaagacttcctccactttagagatgaaagactaagaaggtttggataatttacccaggttatatggctaattgacacaatttgaaccacaactgagggttttaggaaggagctcagggttattccaccatgctcttttcttttattcttccatgtcacctcctcccttcttccatccttcccttctttctttcttgcttcctttacatgtgcatggagtttttaccattactctaaaactatcttaagcactggaattatgaagataaagagacactgacaataaaagctcaaattttatgatttaaaaaaaaagcttttaagttcaggagtacaagtgcaggttgttacataggtaaacttttgtcatggaagtttgttgtacagattatttcatcattcaggtattaatcctagtactcactggttgtttttcctgattctctccctcctcccaccgtccaccctctgaaaggctccagtgtgtgttgttcgcctctctgtgtccatgtgttctcattgttcacctctcacttatgagtgagaacatgtggtgtttggttttctgttcttgtgttagtttgctgaggatgatagcttccagcttaattcatgtccctgtaaaggacatgactttattcttttttatggctatatagtttccatggtgtacatgtcccacattttttttatccagtctattattgatgggcatttaggttgaatccatgtctttgctattgtgcataatgctgtaatgaacatacacatgcatgtgctattatagtagaatgatttatattcctttgggtatatacccagtcatgggattgctgggttgaatggtatttctgtttttaggtcttcgaggaattgccacactgtcttccacaatggctgaactggtttacattcacaccaacagtgtataagtattcttttttctccacaacctcaccagcatctgttatttttttgactttttaataatagccattatgactggtatgagatgatatatcattgtggttttagtttttaatttcccggctgggcacggtggctcatgcctttaatcccagcactttgggaggctgaggcaagtggatcatgaagtcaggagaatgagaccatcctgactaacatggtgaaaccccgtctctactaaaaatacaaaaaattagccaggtgtggtggcgggcacctgtagtcccagctactcgggaggctgaggcaggagaatggcatgaacctgggaggcggagcttgcagtgagctgagatcccgccactgcactccagcctgggtggaagagcgagactccatttcaaaaaagaaaaaaaggaatttctctagtaattagtgatggttagcttttttcatatgattgttggccacacatatgtcttcttttgaaaagtgtctgttcatgtcttttgccyacttttttttgtggggtcgtttgttttctttttcttgtcaatttgcttatgttccttatagatactggacattagacctttgtcagatatagagtttgcaaaattttctcccattttttaggttgtttctcccattttgtaggtttactctgttgatagtttcttttgctgtgcagaaactctttagtttaattatatcccatttagcaatgttttgctttagttgcaattgctgtgggtgccttcatcatgaaatctttgcctgtgtctgtgtcctaaatggtattgcctaggttgtcttccagggttttcatagtttcaagttttacatttaaatctttagtccatcttgaataaatttgtgtatgtggcataaggaatgggttcagtttcagtcttctacatatggctagccagttatcccactaccatttattgaatcaggagtcatttctccattgcttgtttctgtcacctttgttgaatatcagatagttgcaggagtgtggtcttttttctgggttctctattctgttctctatttgtctatgtgtctgctttttcaccggtaccttgctgtgttggttactgtagccttgtactatactttgaagtcaggtagcatgatgcctccagctttgttctttttgcttaggattgccttggctatgcaggtcttatttggttccatatgcattttaaaatagttttctctaattctgtgaagaatgtcaatggtagttgaataggaatagcattgagtctataaattgctttgggcagtatgaccattttaatgatattgattcttcctatccatgagcatggaatgtttttctatttgtttgtgtcatctctgacttctttgagcaggggtttgtagttttccttgtagatctcttccacctacctagtcagctgtattcataggtattttattctttatgtggcattatagtttcttccttgattttgttcttagcttgactgttgttggtgtataggaatgctaataattttttcacattgattttatatcctgagactttgctgaagttgtcagcttaagaagcctttgggctgagactatggggttttctagatatagaatcatgttatccacaaacaggggtggtttgacttcctctctttctatttgtttatttatttctcttgcctgattgccctggccagaactttcgatattgtattaaataggaggagtgagagagggtatccttgtcttgtgacaatttcaaggggaatgcttccagctattgcttctattgagatattaataatcatgtaatttttgcctttagttctgtttatgtgatgaatcacatttatttacttgcatatatttaaccaaccttgtttcctggaaatgaagcctacttgatcataatggataagctttttgatgttctgctagatttagtttgctagtatttcattgaggatttttgaatgaacatcaaaaatattgacctgaagttttcttttttgcgtgtgtctctgccagattttggtatcaggatgatactggtctcatagaatgagttagggaggaggtccttctcaattttttggaatagtttcagtaggaatggtaccagctcttctttatacctctagtagaattcagctgtgaatccatctggtcctgggcttgttttgcttgtaggatatttattactgcctcaatttcagaactcattattggtctgttcatggattcagtttcttcctggttcagttttgggagagtccaggaattcatccatttctctagattttctagtttatgagcatagagaaaggccacatcaactaccaagggaagccagtcagactaactgcagagctctcagctgaaattctctatgccagaagagattaggagccaacattcagcattcttaaataaaagaaattataacccagaatttcatatatgaccaaactaagcttcataagcaaaagagaaataagatccttttcagacaagcaaatgctgagggaatttgttaccaccagacctgccttgcaagagctcctcaaggaagcactgaatatgaaaaggaaaggttgttagtagccactacagaaacacactgaagtacacagaccagtgatgctgtaaagcaaccacataaacaagtctgcacaataaccagctaaacatgatgacaagagcaaatccacacatatcaatagtaaccttaaatgtaaataggctaaatgccccaattaaatgacacagaatggcaatctggataaggaaccaagacccattggtatgctgcctctaagagacccatctcacatgctatgacacacataggctcaaaataaagggatggaagaaaatcttccaggcaaatggaaaacagaaaaaaggcaggggttgcaattctaatttttaccaaaacagacttcaaaccaacaaagatcaaaaaaagacaaagaaggtcattacatagtggtaaaaaattcaattcagcaagaagatctaactattgtaaatctatatacttcccacacaggagcacctagattcataaagcaagtttagagacctttgaagagacttacattcccacacaataatagtgggagactttaacacctcactgacaatattagatcatcaagacagaaaattaagatattcaggacctgaacttagcacttaattgaatgggcctgatagatatctatagaactctccaccacaaaacaacagaatatacattcttctcattgccacatggcaattactctaaaattgattacacaatcaaaagtaaaacactcctcagtaaatgcaaaagaactgaaatcatgacagtctcttgtaccacagcacaatcaaatttgaaatttagactaagaaattcactcaaaattatacaattatgtggaaattgaataacctgctcctgaatgacatgataaacaataaaattatgcagaattcaagaagtttttgaaactaatgagaacaaagataccatgtcccacaatgtctgggacacagctaaggaagtgttaagagggaaatttgtagcactaaatgaccacatcaaaaagttagaaagatctgaagttattataacaacataatatcacaactaaaagaattagagaaccaagagcaaagaaatgccaaagctagcagaagacaagaaataaccagcatcagaactgaactggaggagagagacatgaaaaacattcataaaatcaatgaatccaggagctgtttttttgaaaaaaattaataaatagaccactagctagactaataaagagaaaaagagaaaattcaaataaacacagtaaaaaatgacaatgggaatattaccactgaccccacagaaatacctattttatggttttttaaaatgcaaataaaaaatttgaaacttatccagagagaagtctaattaagcaatgcccttaaactgtgcataaagagcacccttcccagccttaaagccatgctgggtaatagggtaagaactgctttagtattcaattcagatgttcatcttcaaccacacttaaagagcttctgcaactct

SEQ ID NO: 20 - DNA, 601 bp, Homo sapiens
attgcttttaatctgaccttccatgaagacaagacacatgtgtctgagaataatgtaatacacaccctgaaatgagaagtatgtaaatattacaacacaattaattcaaatataaatatatttagtaatatgcaaatacaaagattttcttagttatattttactttttctagtaaataagagcacttggagcatcctcttctgcattagttggtccttctttatgtgttcagcatttttcttggttcatacctatcacacattctagagcacttattatttgcagattatttgcagcttrtggtaatatgtgatccaaatgccatgccaagtgacactgaaaaattactattaatgttactcttttacaaataaacttcttcttttacctgaatatgcttcatctgtgttttctctgttgacattataaatgtgtttctccatcttgagataaaccaatgacattttacactttttgataagaagagtcacttcctgagtgggatggaagctaatgataagtttgttttggaatgatggatcttatcatatgttgtaggcaaaagagtcataatacaccctccatcaccacatttatcat

SEQ ID NO: 21 - DNA, 501 bp, Homo sapiens
tactgaaaattgcaaagcataaactgaaaatgaccattggcttggataaagtcataaagtcataagggaattctatgtttccagatcacaatacctattaaagtaagtaaaatgagtcacttcttacattatgcattttcttcttttttatttcagccatatttttattggggacgggttacatttatgtaccagtgcataagtagacaatgatgcttggtgactatatcctttttattttctaccctctyttctcctgcccttgctctcttcaatttgcaaaaggaatatcattacaacccattgaaggacctattcaaattgaacccaaattcaaattagtaatgtacattttctgaaaaatgaaagaatttaagattaagttttacttttttgccacattagttacataacaaatagagaataagcattaaaaaccacaggacacaaaattcttgaatcaaaacagagatgcagagaagctttatatctaagtcatac

(19) United States
(12) Patent Application Publication
(10) Pub. No.: US 2011/0294803 A1
(43) Pub. Date: Dec. 1, 2011

(54) PHOSPHATIDYLINOSITOL-3-KINASE P110 DELTA-TARGETED DRUGS IN THE TREATMENT OF CNS DISORDERS

(76) Inventors: Amanda J. Law, Washington, DC (US); Daniel R. Weinberger, Washington, DC (US)

(21) Appl. No.: 13/132,425

(22) PCT Filed: Dec. 4, 2009
(86) PCT No.: PCT/US09/66867

§ 371 Date: Aug. 17, 2011

(30) Foreign Application Priority Data
Dec. 4, 2008 (US) 61119978

(51) Int. Cl. A61K 31/5377; A61K 31/506; A61K 31/427; A61K 31/519; A61P 25/18; A61K 31/4985; C12Q 1/68; G01N 33/566; C12Q 1/02; A61P 25/00; A61K 31/52; A61K 31/498

(52) U.S. Cl. 514/234.2; 514/263.21; 514/263.22; 514/275; 514/371; 514/262.1; 514/249; 514/234.8; 435/6.11; 435/7.1; 435/29

(57) ABSTRACT

Methods for treating CNS disorders such as schizophrenia, psychosis and cognitive disorders using specific inhibitors of phosphatidylinositol-3-kinase p110 delta (PIK3CD) expression and/or activity are described. Methods of determining risk of CNS disorders and methods of determining treatment response are also described. An integrative systems biology approach to identify a signaling mechanism and genetic network associated with schizophrenia and with schizophrenia-associated risk variation in ErbB4. A risk pathway associated with ErbB4 genetic variation involving increased expression of a PI3K-linked ErbB4 receptor CYT-1 and a specific PI3K enzyme, PIK3CD has been identified.

### RELATED APPLICATIONS AND PRIORITY CLAIM

This application claims priority to U.S. Provisional Patent Application Ser. No. 61/119,978 filed on Dec. 4, 2008. The application is incorporated herein by reference in its entirety.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. The Government may have certain rights in this invention.

### BACKGROUND

Schizophrenia is a complex, heritable psychiatric disorder. Recently, several putative schizophrenia susceptibility genes have been identified, including neuregulin 1 (NRG1), a gene with pleotropic roles in neurodevelopment and plasticity. Alterations in NRG1 expression and NRG1-mediated signaling have been identified as putative molecular mechanisms mediating the influence of NRG1 upon schizophrenia risk. The NRG1 receptor is ErbB4, a member of the ErbB subfamily of type I receptor tyrosine kinases that regulate cell growth, proliferation and differentiation as a candidate risk gene for schizophrenia. Molecular genetic studies in separate populations have identified specific DNA variants in the ErbB4 gene that are directly linked with risk for the disease, prompting the hypothesis that other molecules in the NRG1 signaling pathway may be involved in the disorder.

The ErbB4 protein is linked to the PI3K pathway. PI3K are members of a unique and conserved family of intracellular lipid kinases that phosphorylate the 3′-hydroxyl group of phosphatidylinositol upon stimulation of growth factor receptor tyrosine kinases. This event leads to the activation of many intracellular signaling pathways that regulates functions as diverse as cell metabolism, survival migration, polarity, and vesicle trafficking and has itself been identified as a potential risk gene for the disease. The observation of increased expression of ErbB4 variants that activate the PI3K pathway suggest altered PI3K signaling in schizophrenia. It is also noteworthy, that PI3K activation results in the recruitment and activation of other signaling molecules, including Rac GTPase, which plays critical roles in neuronal growth, differentiation, migration and intracellular vesicular trafficking by regulation of the actin cytoskeleton. These observations suggest that a number of downstream signaling pathways may be affected in schizophrenia as a consequence of aberrant NRG1/ErbB4 signaling.

What is needed are additional drug targets for the treatment of CNS disorders such as schizophrenia and cognitive dysfunction.

### SUMMARY

In one embodiment, a method for treating a patient in need of treatment for a CNS disorder comprises administering to the patient a therapeutically effective amount of a selective PIK3CD inhibitor, and thereby reducing a symptom of the CNS disorder in the patient.

Disclosed herein are methods of determining increased risk for a CNS disorder in a human.

In an embodiment, the method comprises determining in a nucleic acid sample from a human a nucleotide base at the polymorphic site rs6540991 is a thymine (T), a nucleotide base at the polymorphic site rs9430220 is a thymine (T); a nucleotide base at the polymorphic site rs12567553 is an adenine (A), a nucleotide base at the polymorphic site rs9694151 is an adenine (A), a nucleotide base at the polymorphic site rs6660363 is an adenine (A) a nucleotide base at the polymorphic site rs4601595 is a guanine (O), a nucleotide base at the polymorphic site rs12037599 is a guanine (G), a nucleotide base at the polymorphic site rs1135427 is a thymine (T), a nucleotide base at the polymorphic site rs1141402 is a guanine (G), a nucleotide base at the polymorphic site rs12567553 is an adenine(A), or a nucleotide base at the polymorphic site rs9694151 is an adenine(A); and determining that the human has an increased risk for a CNS disorder.

In an embodiment, the method comprises determining in a nucleic acid sample from a Caucasian the genotype at the polymorphic site rs11589267 is TC; and determining that the Caucasian having the determined genotype TC has an increased risk for a CNS disorder.

In an embodiment, the method comprises determining in a nucleic acid sample from a human the genotype of each polymorphic site in a pair of polymorphic sites, wherein the determined genotypes in the pair of polymorphic site is AA at the polymorphic site rs707284 and TT at the polymorphic site rs4601595, G carrier at the polymorphic site rs839539 and A carrier at the polymorphic site rs11801864, T carrier at the polymorphic site rs1098059 and A carrier at the polymorphic site rs11801864, AA at the polymorphic site rs7598440 and GG at the polymorphic site rs4601595, TT at the polymorphic site rs839541 and GG at the polymorphic site rs12037599, T carrier at the polymorphic site rs1098059 and G carrier at the polymorphic site rs12567553, C carrier at the polymorphic site rs62185768 and CC at the polymorphic site rs9430635, or C carrier at the polymorphic site rs62185768 and AA at the polymorphic site rs6660363; and determining that the human having the determined genotypes in the pair of polymorphic sites has an increased risk for a CNS disorder.

Also disclosed herein are methods of determining treatment response of a patient to a PIK3CD inhibitor.

In an embodiment, the method comprises determining in a nucleic acid sample from a patient with a CNS disorder a nucleotide base at the polymorphic site rs6540991 is a thymine (T), a nucleotide base at the polymorphic site rs9430220 is a thymine (T); a nucleotide base at the polymorphic site rs12567553 is an adenine (A), a nucleotide base at the polymorphic site rs9694151 is an adenine (A), a nucleotide base at the polymorphic site rs6660363 is an adenine (A), a nucleotide base at the polymorphic site rs4601595 is a guanine (G), a nucleotide base at the polymorphic site rs12037599 is a guanine (G), a nucleotide base at the polymorphic site rs1135427 is a thymine (T), a nucleotide base at the polymorphic site rs1141402 is a guanine (G); a nucleotide base at the polymorphic site rs12567553 is an adenine(A), or a nucleotide base at the polymorphic site rs9694151 is an adenine(A); and determining that the patient having the determined nucleotide base is likely to respond to treatment with an effective amount of a PIK3CD inhibitor.

In an embodiment, the method comprises determining in a nucleic acid sample from a Caucasian the genotype at the polymorphic site rs11589267 is TC; and determining that the Caucasian having the determined genotype TC is likely to respond to treatment with an effective amount of a selective PIK3CD inhibitor.

In an embodiment, the method comprises determining in a nucleic acid sample from a patient with a CNS disorder the genotype of each polymorphic site in a pair of polymorphic sites, wherein the determined genotype s in the pair of polymorphic site is AA at the polymorphic site rs707284 and TT at the polymorphic site rs4601595, G carrier at the polymorphic site rs839539 and A carrier at the polymorphic site rs11801864, T carrier at the polymorphic site rs1098059 and A carrier at the polymorphic site rs11801864, AA at the polymorphic site rs7598440 and GG at the polymorphic site rs4601595, G carrier at the polymorphic site rs839539 and A carrier at the polymorphic site rs7518793, TT at the polymorphic site rs839541 and GG at the polymorphic site rs12037599 T carrier at the polymorphic site rs1098059 and G carrier at the polymorphic site rs12567553, C carrier at the polymorphic site rs62185768 and CC at the polymorphic site rs9430635, or C carrier at the polymorphic site rs62185768 and AA at the polymorphic site rs6660363; and determining that the patient having the determined genotypes in the pair of polymorphic sites is likely to respond to treatment with an effective amount of a selective PIK3CD inhibitor.

In an embodiment, the method comprises determining in a biological sample from a patient with a CNS disorder an expression level of a gene that is greater than expression level of the gene determined for a control population lacking the CNS disorder, wherein the gene is PIK3CD or ErbB4, or determining in a biological sample from a patient with a CNS disorder a level of NRG1-induced phosphatidylinositol-3,4,5-triphosphate ([PI(3,4,5)P3] production or NRG1-induced cell migration that is smaller than the level for a control population lacking the CNS disorder; and determining that the patient is likely to respond to treatment with an effective amount of a selective PIK3CD inhibitor.

### BRIEF DESCRIPTION OF THE DRAWINGS

FIG. 1 shows experimental results implicating PIK3CD in a CNS disorder, wherein panel (A) shows expression of class IA phosphatidylinositol-3-kinase (PI3K) genes in human LCLs measured by quantitative real-time RT-PCR in normal control subjects (n=32) and patients with schizophrenia (SZ; n=23); panel (B) shows normalized PIK3CD and PI3KR3 expression in normal and patient-derived LCLs as a function of diplotype for the ErbB4 risk haplotype (AGG/AGG, n=13; AGG/non risk, n=28, non risk/non risk, n=14); panel (C) shows a graph of NRG1-induced [PI(3,4,5)P3] production in LCLs as a function of diplotype of the ErbB4 risk haplotype in the whole sample, with the inset showing a graph of the data parsed by diagnostic group (darker bars are patients with schizophrenia); panel (D) compares NRG1-induced [PI(3,4,5)P3] production in controls and in patients with schizophrenia; panel (E) shows a graph of chemotaxis to NRG1 as a function of diplotype of the ErbB4 risk haplotype in the whole sample, with the inset showing the data parsed by diagnosis; and panel (F) shows a graph of chemotaxis to NRG1 as a function of NRG1-induced [PI(3,4,5)P3] production (n=47); all values are means showing the standard error of the mean (SEM).

FIG. 2 presents histograms of normalized PIK3CD or PIK3R3 mRNA expression (mean±SEM); panel (A) shows a histogram of PIK3CD mRNA expression of normal controls as a function of diplotype of the ErbB4 risk haplotype in dorsolateral prefrontal cortical grey matter (DLPFC) and hippocampus of normal controls; panel (B) presents a histogram of PIK3R3 mRNA expression as a function of disease state (control or schizophrenia) in DLPFC and hippocampus; panel (C) presents a histogram of PIK3CD mRNA expression as a function of disease state (control or schizophrenia) in DLPFC and hippocampus; panel (D) shows expression of PIK3CD mRNA in the hippocampus of rats treated with haloperidol (0, 0.08, and 0.6 mg/kg/day).

FIG. 3 shows a schematic representation of the PIK3CD gene region (center) superimposed with association test results in the CBDB SS sample and NIMHGI-AA family cohorts for single SNPs and sliding window 3 SNP haplotypes above the PIK3CD gene representation and linkage disequilibrium (LD, as r²) results between PIK3CD SNP loci for 370 unrelated healthy Caucasian controls below the PIK3CD gene representation.

FIG. 4 illustrates genotype-based differences in DLPFC activation during the N-back working memory task (2-back) in control subjects for PIK3CD rs9430635 (A and B) and ErbB4 rs7598440 (C and D). A) Threshold statistical t-map of DLPFC activation (2-back-0-back) rendered on the MNI brain template for PIK3CD rs9430635 (p<0.001); B) Genotype effect on task related mean BOLD signal change in the left DLPFC (MNI coordinates of peak cluster: x=−34, y=41, z=41 mm); C) Threshold statistical t-map of DLPFC activation related to task (2-back-0-back) rendered on the MNI brain template for ErbB4 rs7598440 (p<0.001); D) Genotype effect on task related mean BOLD signal change in the right DLPFC (MNI coordinates of peak cluster: x=52, y=34, z=26 mm); Bars represent Mean±SEM extracted BOLD signal change from peak clusters in each genotype group normalized to the mean of the GG genotype group (rs7598440) or the CC group (rs9430635).

FIG. 5A shows a graph of chemotaxis to NRG1 as a function of the genotype of a schizophrenia-associated PIK3CD polymorphism in LCLs of patients with schizophrenia; and FIG. 5B shows a graph of PIK3CD protein levels as a function of the genotype of a schizophrenia-associated PIK3CD polymorphism in LCLs of patients with schizophrenia.

FIG. 6A shows a graph of NRG1 stimulated [PI(3,4,5)P3] production as a function of PIK3CD mRNA human LCLs (N=55); FIG. 6B shows a graph of NRG1 stimulated [PI(3,4,5)P3] production as a function of PIK3CD protein expression human LCLs; and FIG. 6C shows a graph of cell migration as a function of PIK3CD protein level in human LCLs.

FIG. 7 shows the effect of IC87114 on chemotaxis to NRG1 in human LCLs of normal individuals and patients with schizophrenia, in vitro. A) Individual response data; B) ANOVA group mean effect of IC87114. N=32.

FIG. 8 shows that IC87114 treatment reduces amphetamine-induced hyperlocomotion in mice (1.5 mg/kg amphetamine).

FIG. 9 shows that IC87114 treatment has no effect on baseline locomotor activity in mice.

FIG. 10 shows that IC87114 treatment dramatically reduces amphetamine-induced stereotypy in a genetic mutant model of schizophrenia.

The above-described and other features will be appreciated and understood by those skilled in the art from the following detailed description, drawings, and appended claims.

### DETAILED DESCRIPTION

The compositions and methods disclosed herein are based, at least in part, on the discovery that a specific isoform of the phosphatidylinositol-3-kinase p110 catalytic subunit, phosphatidylinositol-3-kinase p110 delta, (also referred to as “PIK3CD” or “PI3K delta” or “PI3K δ”) is critically involved in CNS disorders such as schizophrenia and those relating to human cognition, and is a target for the treatment of CNS disorders such as psychosis and cognitive dysfunction. Accordingly, disclosed herein are compositions and methods for treating CNS disorders such as schizophrenia and cognitive disorders using selective inhibitors of PIK3CD expression and/or activity.

The inventors herein have discovered that: 1) variations in the genetic sequence for the PIK3CD gene are associated with genetic risk for schizophrenia in Caucasian and African American family samples; 2) variation in the genetic sequence of PIK3CD also affects many aspects of normal human cognitive functions, including memory, IQ, and executive cognition; 3) PIK3CD expression is increased in the blood of patients with schizophrenia, and the level of its expression in blood and human brain is predicted by variation in the gene ErbB4, a receptor responsible for the direct upstream activation of PIK3CD. ErbB4 also is related to risk for schizophrenia and cognition and interacts genetically with PIK3CD to further increase risk; 4) traditional antipsychotic drugs when given to rodents reduce the expression of PIK3CD in the brain, indicating that they target this protein; 5) a drug that specifically inhibits PIK3CD rescues a cellular phenotype related to schizophrenia. The cellular phenotype is migration of lymphocytes to the chemoattractant, Neuregulin (NRG1), a key regulator of brain development. NRG1 induced lymphocyte migration is diminished in patients with schizophrenia, and is predicted by schizophrenia associated genetic variation in PIK3CD and in other genes that directly activate PIK3CD; and 6) PIK3CD inhibitors decrease amphetamine induced locomotor abnormalities in a genetic mouse model of schizophrenia.

In one aspect, disclosed herein are methods of ameliorating or preventing CNS disorders by administering to an individual an amount of a selective PIK3CD inhibitor effective to ameliorate or prevent CNS disorders and PIK3CD activity. In one embodiment, the methods include inhibiting PIK3CD enzymatic activity directly, and in another embodiment, methods include inhibiting PIK3CD enzymatic activity by inhibiting PIK3CD expression.

Phosphatidylinositol-3-kinase was originally identified as an activity associated with viral oncoproteins and growth factor receptor tyrosine kinases that phosphorylate phosphatidylinositol (PI) and its phosphorylated derivatives at the 3′-hydroxyl of the inositol ring. Phosphatidylinositol-3-kinase activation, therefore, is believed to be involved in a range of cellular responses including cell growth, differentiation, and apoptosis. Four distinct Class I PI3Ks have been identified, designated PI3K α, β, δ, and γ, each consisting of a distinct 110 kDa catalytic subunit and a regulatory subunit. Three of the catalytic subunits, i.e., p110α, p110β, and p110δ, each interact with the same regulatory subunit, p85; whereas p110γ interacts with a distinct regulatory subunit, p101. Details concerning the P110δ isoform also can be found in U.S. Pat. Nos. 5,858,753; and 5,985,589, incorporated herein by reference for their teaching of the sequence of PIK3CD and methods of testing for inhibitors of PIK3CD.

The term “selective PIK3CD inhibitor” as used herein refers to a compound that inhibits the PIK3CD isozyme more effectively than other isozymes of the PI3K family. A “selective PIK3CD inhibitor” is understood to be more selective for PIK3CD than compounds conventionally and generically designated PI3K inhibitors, e.g., wortmannin or LY294002. Wortmannin and LY294002 are “nonselective PI3K inhibitors”.

The relative efficacies of compounds as inhibitors of a biological activity can be established by determining the concentrations at which each compound inhibits the activity to a predefined extent, then comparing the results. Typically, the concentration that inhibits 50% of the activity in a biochemical assay is determined, i.e., the 50% inhibitory concentration or “IC50”. IC50 determinations can be accomplished using conventional techniques known in the art. For example, an IC50 can be determined by measuring the biological activity in the presence of a range of concentrations of the inhibitor under study. The experimentally obtained values of enzyme activity then are plotted against the inhibitor concentrations used. The concentration of the inhibitor that shows 50% biological activity (as compared to the activity in the absence of any inhibitor) is taken as the IC50 value. Analogously, other inhibitory concentrations can be defined through appropriate determinations of activity. For example, in some settings it can be desirable to establish a 90% inhibitory concentration, i.e., IC90.

In one embodiment, PIK3CD inhibitors exhibit an IC50 value vs. human PIK3CD of about 10 μM or less. In several embodiments, the compounds have an IC50 vs. human PIK3CD of less than 5 μM. In other embodiments, the compounds have an IC50 value vs. human PIK3CD of less than 1 μM, less than 100 nM, less than 10 nM or less than 1 nM.

Accordingly, a “selective PIK3CD inhibitor” can be understood to refer to a compound that exhibits an IC50 with respect to human PIK3CD that is at least 2-fold, at least 5-fold, at least 10-fold, specifically at least 20-fold, and more specifically at least 30-fold, lower than the IC50 value with respect to any or all of the other Class I PI3K family members. The term “specific PIK3CD inhibitor” can be understood to refer to a selective PIK3CD inhibitor that exhibits an IC50 with respect to human PIK3CD that is at least 50-fold, specifically at least 100-fold, more specifically at least 200-fold, and still more specifically at least 500-fold, lower than the IC50 with respect to any or all of the other PI3K Class I family members.

In certain embodiments, a selective PIK3CD inhibitor exhibits an IC50 with respect to human PI3K alpha that is at least 5, 10, 20 or 50 times the IC50 with respect to human PIK3CD and human PI3K gamma; and exhibits an IC50 with respect to human PI3K beta that is at least 2, 5, 10 or 20 times the IC50 with respect to human PIK3CD.

Methods for determining the IC50 of a PIK3CD inhibitor include contacting a PIK3CD polypeptide with a test compound and measuring the affinity of the inhibitor for the PIK3CD polypeptide and/or measuring the effect of the polypeptide on the activity of the PIK3CD polypeptide. For confirming selectivity, PI3K polypeptides corresponding to other isoforms are used. Suitable assays are well known in the art and include, for example, assays that determine inhibition of SCF-induced Akt phosphorylation in mast cells. Briefly, mast cells that are stored in medium containing no SCF or IL-3 are preincubated with test compound (e.g., for 20 minutes), cells are activated with SCF (e.g., 20 ng/mL, for 15 minutes at 37° C.). Cells are fixed and permeabilized and Akt phosphorylation visualized using phospho-Ser-473 specific Akt antibodies and standard FACS protocols.

In these methods, PIK3CD polypeptides include full length PIK3CD, as well as fragments of PIK3CD that exhibit kinase activity, i.e., a fragment comprising the catalytic site of PIK3CD. Alternatively, the PIK3CD polypeptide is a fragment from the PIK3CD-binding domain of p85 and provides a method to identify allosteric modulators of PIK3CD. The methods can be employed in cells expressing cells expressing PIK3CD or its subunits, either endogenously or exogenously. Accordingly, the polypeptide employed in such methods can be free in solution, affixed to a solid support, modified to be displayed on a cell surface, or located intracellularly. The modulation of activity or the formation of binding complexes between the PIK3CD and the agent being tested then can be measured.

In one embodiment, the IC50 of a PIK3CD inhibitor is determined in a high-throughput assay. PIK3CD catalyzes a phosphotransfer from γ-[³²P]ATP to phosphatidylinositol 4,5-bisphosphate/phosphatidylserine (PIP2/PS) liposomes at the D3′ position of the PIP2 lipid inositol ring. This reaction is MgCl₂ dependent and is quenched in high molarity potassium phosphate buffer pH 8.0 containing 30 mM EDTA. In the screen, this reaction is performed in the presence or absence of inhibitory compounds. The reaction products (and all unlabelled products) are transferred to a 96-well, prewetted PVDF filter plate, filtered, and washed in high molarity potassium phosphate. Scintillant is added to the dried wells and the incorporated radioactivity is quantitated.

The terms “blocker”, “inhibitor”, or “antagonist” are used interchangeably to mean a substance that retards or prevents a chemical or physiological reaction or response. Exemplary blockers or inhibitors comprise, but are not limited to, antisense molecules, siRNA molecules, antibodies, small molecule antagonists, and their derivatives. A PIK3CD blocker or inhibitor inhibits the activity and/or concentration of PIK3CD.

Compounds of Formula I, including the pharmaceutically acceptable salts and/or hydrates thereof, are disclosed as selective PIK3CD inhibitors in U.S. Pat. No. 6,667,300 and U.S. Patent Application Publication No. 2009/0270426, and compound descriptions and methods of preparation therein are hereby incorporated by reference.

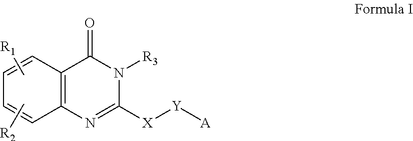

In Formula I:

A is an optionally substituted monocyclic 5-membered heterocyclic ring containing two or three nitrogen atoms or a bicyclic ring system containing two nitrogen atoms and one ring of the bicyclic system is aromatic;

X is C(R_{b})₂, CH₂CHR_{b}, or CH═C(R_{b});

Y is S, SO, or SO₂;

R₁ and R₂, independently, are selected from hydrogen, C₁₋₆ alkyl, aryl, heteroaryl, halo, NHC(═O)C₁₋₃ alkyleneN(Rₐ)₂, NO₂, ORₐ, CF₃, OCF₃, N(Rₐ)₂, CN, OC(═O)Rₐ, C(═O)Rₐ, C(═O)ORₐ, arylOR_{b}, Het, NRₐC(═O)C₁₋₃allyleneC(═O)ORₐ, C(═O)ORₐ, C₁₋₃alkyleneN(Rₐ)₂, arylOC(═O)Rₐ, C₁₋₄alkyleneC(—O)ORₐ, OC₁₋₄alkyleneC(═O)ORₐ, C₁₋₄alkyleneOC₁₋₄alkyleneC(═O)ORₐ, C(═O)NRₐSO₂Rₐ, C₁₋₄alkyleneN(Rₐ)₂, C₂₋₆alkenylene-N(Rₐ)₂, C(═O)NRₐC₁₋₄alkyleneOltₐ, C(═O)NRₐC₁₋₄alkylene-Het, OC₂₋₄alkyleneN(Rₐ)₂, OC₁₋₄alkyleneCH(OR_{b})CH₂N(Rₐ)₂, OC₁₋₄alkyleneHet, OC₂₋₄alkyleneORₐ, OC₂₋₄alkylene-NRₐC(—O)ORₐ, NRₐ C₁₋₄alkyleneN(Rₐ)₂, NRₐC(═O)Rₐ, NRₐC(═O)N(Rₐ)₂, N(SO₂C₁₋₄alkyl)₂, NRₐC(SO₂C₁₋₄ alkyl), SO₂N(Rₐ)₂, OSO₂CF₃, C₁₋₃alkylenearyl, C₁₋₄alkyleneHet, C₁₋₆alkyleneOR_{b}, C₁₋₃alkyleneN(Rₐ)₂, C(═O)N(Rₐ)₂, NHC(═O)C₁-C₃alkylenearyl, C₃₋₈cycloalkyl, C₃₋₈heterocycloalkyl, arylOC₁₋₃alkyleneN(Rₐ)₂, arylOC(—O)R_{b}, NHC(═O)C₁₋₃alkyleneC₃₋₈heteroeycloalkyl, NHC(═O)C₁₋₃alkyleneHet, OC₁₋₄alkyleneOC₁₋₄alkyleneC(═O)OR_{b}, C(═O)C₁₋₄alkyleneHet, and NHC(═O)haloC₁₋₆alkyl;

R₃ is optionally substituted aryl;

each Rₐ is selected from hydrogen, C₁₋₆alkyl, C₃₋₈cycloalkyl, C₃₋₈heterocycloalkyl, C₁₋₃alkyleneN(R_{c})₂, aryl, arylC₁₋₃alkyl, C₁₋₃alkylenearyl, heteroaryl, heteroarylC₁₋₃alkyl, and C₁₋₃alkyleneheteroaryl;

or two Rₐ groups are taken together to form a 5- or 6-membered ring, optionally containing at least one heteroatom;

each R_{b} is selected from hydrogen, C₁₋₆alkyl; R_{c} is selected from hydrogen, C₁₋₆alkyl, C₃₋₈cycloalkyl, aryl, and heteroaryl; and

each Het is selected from 1,3-dioxolane, 2-pyrazoline, pyrazolidine, pyrrolidine, piperazine, pyrroline, 2H-pyran, 4H-pyran, morpholine, thiomorpholine, piperidine, 1,4-dithiane, and 1,4-dioxane, and optionally substituted with C₁₋₄alkyl or C(═O)ORₐ.

Certain compounds of Formula I further satisfy Formula Ia or Formula Ib:

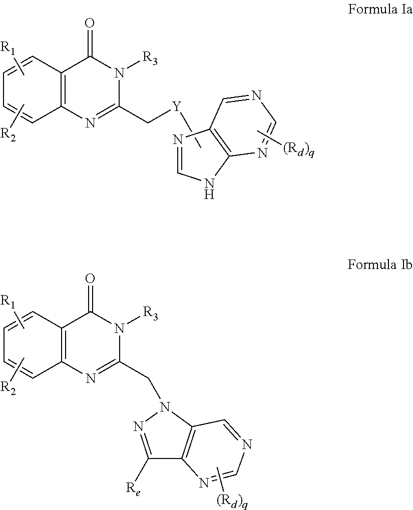

wherein:

R₁ is absent or is a substituent selected from halo, NO₂, OH, OCH₃, CH₃, and CF₃;

R₂ is absent or is a substituent selected from halo, and OCH₃;

or R₁ and R₂ together with C₁₋₆ and C-7 of the quinazoline ring system define a 5- or 6-membered aromatic ring optionally containing one or more heteroatom ring members independently chosen from O, N, and S;

R₃ is C₁-C₆alkyl, phenyl, biphenyl, benzyl, pyridinyl, piperazinyl, C(═O))R₄ or morpholinyl; each of which is unsubstituted or substituted with from 0 to 3 substituents independently chosen from halo, C₁-C₆alkyl, C₁-C₆alkoxy; wherein R₄ is C₁-C₆alkyl;

Y is absent, S or NH; such that the purine moiety is linked via a carbon or nitrogen atom present on either ring;

each R_{d} and Rₑ are independently chosen from NH₂, halo, C₁-C₃alkyl, S(C₁-C₃alkyl), OH, NH(C₁-C₃alkyl), N(C₁-C₃alkyl)₂, NH(C₁-C₃alkylenephenyl)

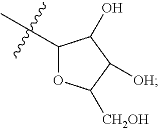

and

Q is 1 or 2.

Representative compounds of Formula I include, but are not limited to:
3-(2-isopropylphenyl)-5-methyl-2-(9H-purin-6-ylsulfanylmethyl)-3H-quinazolin-4-one;
5-chloro-2-(9H-purin-6-ylsulfanylmethyl)-3-o-tolyl-3H-quinazolin-4-one;
5-chloro-3-(2-fluorophenyl)-2-(9H-purin-6-ylsulfanylmethyl)-3H-quinazolin-4-one;
3-(2-fluorophenyl)-5-methyl-2-(9H-purin-6-ylsulfanylmethyl)-3H-quinazolin-4-one;
3-(2-methoxyphenyl)-5-methyl-2-(9H-purin-6-ylsulfanylmethyl-3H-quinazolin-4-one;
3-(2,6-dichlorophenyl)-5-methyl-2-(9H-purin-6-ylsulfanylmethyl)-3H-quinazolin-4-one;
3-(2-chlorophenyl)-6-fluoro-2-(9H-purin-6-ylsulfanylmethyl)-3H-quinazolin-4-one;
5-chloro-3-(2-chlorophenyl)-2-(9H-purin-6-ylsulfanylmethyl)-3H-quinazolin-4-one;
3-(2-chlorophenyl)-5-methyl-2-(9H-purin-6-ylsulfanylmethyl)-3H-quinazolin-4-one;
3-(3-methoxyphenyl-2-(9H-purin-6-ylsulfanylmethyl-3H-quinazolin-4-one;
3-(2-chlorophenyl)-5-fluoro-2-(9H-purin-6-ylsulfanylmethyl)-3H-quinazolin-4-one;
3-benzyl-2-(9H-purin-6-ylsulfanylmethyl)-3H-quinazolin-4-one;
3-butyl-2-(9H-purin-6-ylsulfanylmethyl)-3H-quinazolin-4-one;
3-(2-chlorophenyl)-7-fluoro-2-(9H-purin-6-ylsulfanylmethyl)-3H-quinazolin-4-one;
3-morpholin-4-yl-2-(9H-purin-6-ylsulfanylmethyl)-3H-quinazolin-4-one, acetate salt;
8-chloro-3-(2-chlorophenyl)-2-(9H-purin-6-ylsulfanylmethyl)-3H-quinazolin-4-one;
3-(2-chlorophenyl)-6,7-difluoro-2-(9H-purin-6-ylsulfanylmethyl)-3H-quinazolin-4-one;
3-(2-methoxyphenyl-2-(9H-purin-6-ylsulfanylmethyl)-3H-quinazolin-4-one;
6-chloro-3-(2-chlorophenyl)-2-(9H-purin-6-ylsulfanylmethyl)-3H-quinazolin-4-one;
3-(3-chlorophenyl)-2-(9H-purin-6-ylsulfanylmethyl)-3H-quinazolin-4-one;
2-(9H-purin-6-ylsulfanylmethyl)-3-pyridin-4-yl-3H-quinazolin-4-one;
3-(2-chlorophenyl)-2-(9H-purin-6-ylsulfanylmethyl)-trifluoromethyl-3H-quinazolin-4-one;
3-benzyl-5-fluoro-2-(9H-purin-6-ylsulfanylmethyl)-3H-quinazolin-4-one;
3-(4-methylpiperazin-1-yl)-2-(9H-purin-6-ylsulfanylmethyl)-3H-quinazolin-4-one, acetate salt;
3-(2-chlorophenyl)-6-hydroxy-2-(9H-purin-6-ylsulfanylmethyl)-3H-quinazolin-4-one;
[5-fluoro-4-oxo-2-(9H-purin-6-ylsulfanylmethyl)-4H-quinazolin-3-yl]acetic acid ethyl ester;
3-(2,4-dimethoxyphenyl)-2-(9H-purin-6-ylsulfanylmethyl)-3H-quinazolin-4-one;
3-biphenyl-2-yl-5-chloro-2-(9H-purin-6-ylsulfanylmethyl)-3H-quinazolin-4-one;
2-(6-aminopurin-9-ylmethyl)-3-(2-isopropylphenyl)-5-methyl-3H-quinazolin-4-one;
2-(6-aminopurin-9-ylmethyl)-5-methyl-3-o-tolyl-3H-quinazolin-4-one; 2-(6-aminopurin-9-ylmethyl)-3-biphenyl-2-yl-5-chloro-3H-quinazolin-4-one;
5-chloro-3-(2-methoxyphenyl)-2-(9H-purin-6-ylsulfanylmethyl)-3H-quinazolin-4-one;
2-(6-aminopurin-9-ylmethyl)-3-(2-fluorophenyl)-5-methyl-3H-quinazolin-4-one;
2-(6-aminopurin-9-ylmethyl)-5-chloro-3-(2-fluorophenyl)-3H-quinazolin-4-one;
2-(6-aminopurin-9-ylmethyl)-8-chloro-3-(2-chlorophenyl)-3H-quinazolin-4-one;
2-(6-aminopurin-9-ylmethyl)-5-chloro-3-(2-chlorophenyl)-3H-quinazolin-4-one;
2-(6-aminopurin-9-ylmethyl)-3-(2-chlorophenyl)-5-methyl-3H-quinazolin-4-one;
2-(6-aminopurin-9-ylmethyl)-3-(2-chlorophenyl)-5-fluoro-3H-quinazolin-4-one;
2-(6-aminopurin-9-ylmethyl)-3-benzyl-5-fluoro-3H-quinazolin-4-one;
2-(6-aminopurin-9-ylmethyl)-3-butyl-3H-quinazolin-4-one;
2-(6-aminopurin-9-ylmethyl)-3-morpholin-4-yl-3H-quinazolin-4-one;
2-(6-aminopurin-9-ylmethyl)-3-(2-chlorophenyl)-7-fluoro-3H-quinazolin-4-one;
3-(2-chlorophenyl)-2-(9H-purin-6-ylsulfanylmethyl)-3H-quinazolin-4-one;
3-phenyl-2-(9H-purin-6-ylsulfanylmethyl)-3H-quinazolin-4-one;
2-(6-aminopurin-9-ylmethyl)-5-chloro-3-(2-isopropylphenyl)-3H-quinazolin-4-one; and
2-(6-aminopurin-9-ylmethyl)-5-chloro-3-o-tolyl-3H-quinazolin-4-one,

as well as the pharmaceutically acceptable salts and/or hydrates of the foregoing compounds.

Certain selective PIK3CD inhibitors provided herein have the structure:

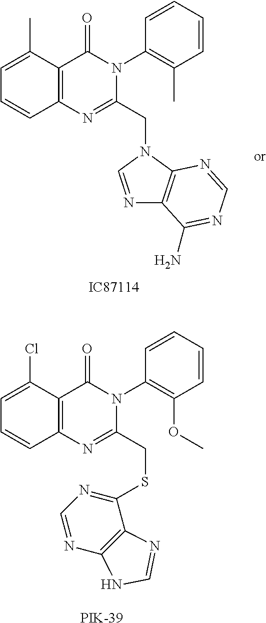

or are a pharmaceutically acceptable salt and/or hydrate thereof.

Compounds of Formula II, including the pharmaceutically acceptable salts and hydrates thereof, are disclosed as selective PIK3CD inhibitors in PCT International Application Publication No. WO 09/064,802, and compound descriptions and methods of preparation therein are hereby incorporated by reference.

### Within Formula II:

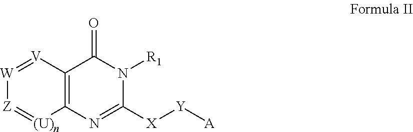

U, V, W, and Z, independently, are selected from CRₐ, N, NR_{b}, and O; or at least one of U, V, W and Z is N, and the others of U, V, W and Z are selected from the group consisting of CRₐ, NR_{b}, S, and O; and at least one, but not all, of U, V, W, and Z is different from CRₐ;

A is an optionally substituted monocyclic or bicyclic ring system containing at least two nitrogen atoms as ring members, and at least one ring of the system is aromatic;

X is C(R_{c})₂, C(R_{c})₂C(R_{c})₂, CH₂CHR_{c}, CHR_{c}CHR_{c}, CHR_{C}CH₂, CH═C(R_{c}), C(R_{c})═C(R_{c}), or C(R_{C})═CH;

Y is absent, S, SO, SO₂, NH, N(R_{c}), O, C(═O), OC(═O), C(═O)O, or NHC(═O)CH₂S;

R₁ is selected from H, substituted or unsubstituted C₁₋₁₀alkyl, substituted or unsubstituted C₂₋₁₀alkenyl, substituted or unsubstituted C₂₋₁₀alkynyl, substituted or unsubstituted C₁₋₆ perfluoroalkyl, substituted or unsubstituted C₃₋₈cycloalkyl, substituted or unsubstituted C₃₋₈heterocycloalkyl, substituted or unsubstituted C₁₋₄alkyleneC₃₋₈cycloalkyl, substituted or unsubstituted aryl, substituted or unsubstituted heteroaryl, substituted or unsubstituted arylC₁₋₄alkyleneOR_{c}, substituted or unsubstituted heteroarylC₁₋₄alkyleneN(R_{d})₂, substituted or unsubstituted heteroarylC₁₋₄alkyleneORₑ, substituted or unsubstituted C₁₋₃alkyleneheteroaryl, substituted or unsubstituted C₁₋₃alkylenearyl, substituted or unsubstituted arylC₁₋₆alkyl, arylC₁₋₄alkyleneN(R_{d})₂, C₁₋₄alkyleneC(═O)C₁₋₄alkylenearyl, C₁₋₄alkyleneC(═O)C₁₋₄alkyleneheteroaryl, C₁₋₄alkyleneC(═O)heteroaryl, C₁₋₄alkyleneC(═O)N(R_{d})₂, C₁₋₆alkyleneOR_{d}, C₁₋₄alkyleneNRₐC(═O)R_{d}, C₁₋₄alkyleneOC₁₋₄alkyleneOR_{d}, C₁₋₄alkyleneN(R_{d})₂, C₁₋₄alkyleneC(═O)OR_{d}, and C₁₋₄alkyleneOC₁₋₄alkyleneC(═O)OR_{d};

each Rₐ is independently selected from H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, C₁₋₃alkylenearyl, substituted or unsubstituted heteroaryl, substituted or unsubstituted heteroarylC₁₋₃allyl, substituted or unsubstituted C₁₋₃alkyleneheteroaryl, halo, NHC(═O)C₁₋₃alkyleneN(R_{d})₂, NO₂, ORₑ, CF₃, OCF₃, N(R₄)₂, CN, OC(═O)R_{d}, C(═O)R_{d}, C(═O)OR_{d}, arylORₑ, NR_{d}C(═O)C₁₋₃alkyleneC(═O)OR_{d}, arylOC₁₋₃alkyleneN(R_{d})₂, arylOC(═O)R_{d}, C₁₋₄alkyleneC(═O)OR_{d}, OC₁₋₄alkyleneC(═O)OR_{d}, C₁₋₄alkyleneOC₁₋₄alkyleneC(═O)OR_{d}, C(═O)NR_{d}SO₂R_{d}, C₁₋₄alkyleneN(R_{d})₂, C₂₋₆ alkenyleneN(R_{d})₂, C(═O)NR_{d}C₁₋₄alkyleneORₑ, C(═O)NR_{d}C₁₋₄alkyleneheteroaryl, OC₁₋₄alkyleneN(R_{d})₂, OC₁₋₄alkyleneCH(OROCH₂N(R_{d})₂, OC₁₋₄alkyleneheteroaryl, OC₂₋₄alkyleneORₑ, OC₂₋₄alkyleneNR_{d}C(═O)OR_{d}, NRₐC₁₋₄alkyleneN(R_{d})₂, NRₐC═O)R_{d}, NRₐC(═O)N(R_{d})₂, N(SO₂C₁₋₄alkyl)₂, NRₐ(SO₂C₁₋₄alkyl), SO₂N(R_{d})₂, OSO₂CF₃, C₁₋₄alkylenearyl, C₁₋₄alkyleneheteroaryl, C₁₋₆alkyleneORₑ, C(═O)N(R_{d})₂, NHC(═O)C₁₋₃alkylenearyl, arylOC₁₋₃alkyleneN(R_{d})₂, arylOC(═O)R_{d}, NHC(═O)C₁₋₃alkyleneC₃₋₈heterocycloalkyl, NHC(═O)C₁₋₃alkyleneheteroaryl, OC₁₋₄alkleneOC₁₋₄alkyleneC(═O)OR_{d}, C(═O)C₁₋₄ alkyleneheteroaryl, and NHC(═O)haloC₁₋₆alkyl;

each R_{b} is independently absent or selected from H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, substituted or unsubstituted arylC₁₋₃alkyl, C₁₋₃alkylenearyl, substituted or unsubstituted heteroaryl, heteroarylC₁₋₃alkyl, substituted or unsubstituted C₁₋₃alkyleneheteroaryl, C(═O)R_{d}, C(═O)OR_{d}, arylORₑ, arylOC₁₋₃alkyleneN(R_{d})₂, arylOC(═O)R_{d}, C₁₋₄alkyleneC(═O)OR_{d}, C₁₋₄alkyleneOC₁₋₄alkyleneC(═O)OR_{d}, C(═O)NR_{d}SO₂R_{d}, C₁₋₄alkyleneN(R_{d})₂, C₂₋₆alkenyleneN(R_{d})₂, C(═O)NR_{d}C₁₋₄alkyleneORₑ, C(═O)NR_{d}C₁₋₄alkyleneheteroaryl, SO₂N(R_{d})₂, C₁₋₃alkylenearyl, C₁₋₄alkyleneheteroaryl, C₁₋₆ alkyleneORₑ, C₁₋₃ alkyleneN(R_{d})₂, C(═O)N(R_{d})₂, arylOC₁₋₃alkyleneN(R_{d})₂, arylOC(═O)R_{d}, and C(═O)C₁₋₄alkyleneheteroaryl;

each R_{c} is independently selected from H, substituted or unsubstituted C₁₋₁₀alkyl, substituted or unsubstituted C₃₋₈cycloalkyl, substituted or unsubstituted C₃₋₈heterocycloalkyl, substituted or unsubstituted C₁₋₄alkyleneN(R_{d})₂, substituted or unsubstituted C₁₋₃alkyleneheteroC₁₋₃alkyl, substituted or unsubstituted arylheteroC₁₋₃alkyl, substituted or unsubstituted aryl, substituted or unsubstituted heteroaryl, substituted or unsubstituted arylC₁₋₃alkyl, substituted or unsubstituted heteroarylC₁₋₃alkyl, C₁₋₃alkylenearyl, substituted or unsubstituted C₁₋₃alkyleneheteroaryl, C(═O)R_{d}, and C(═O)OR_{d};

or two R_{c} on the same atom or on adjacent connected atoms can cyclize to form a ring having 3-8 ring members, which ring is optionally substituted and may include up to two heteroatoms selected from NR_{d}, O, and S as ring members;

each R_{d} is independently selected from H, substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈heterocycloalkyl, substituted or unsubstituted C₁₋₃alkyleneN(Rₑ)₂, aryl, substituted or unsubstituted arylC₁₋₃alkyl, substituted or unsubstituted C₁₋₃alkylenearyl, substituted or unsubstituted heteroaryl, substituted or unsubstituted heteroarylC₁₋₃alkyl, and substituted or unsubstituted C₁₋₃alkyleneheteroaryl;

or two R_{d} groups are taken together with the nitrogen to which they are attached to form a 5- or 6-membered ring, optionally containing a second heteroatom that is N, O, or S;

each Rₑ is selected from H, substituted or unsubstituted C₁₋₆alkyl, substituted or unsubstituted C₃₋₈cycloalkyl, substituted or unsubstituted aryl, and substituted or unsubstituted heteroaryl,

or two Rₑ groups are taken together with the nitrogen to which they are attached to form a 5- or 6-membered ring, optionally containing a second heteroatom that is N, O, or S;

wherein A, R₁, Rₐ, R_{b}, R_{c}, and R_{d}, independently, are optionally substituted with one to three substituents selected from C₁₋₁₀alkyl, C₂₋₁₀alkenyl, C₂₋₁₀alkynyl, C₃₋₈cycloalkyl, C₃₋₈heterocycloalkyl, C₁₋₆alkyleneORₑ, C₁₋₄alkyleneN(Rₑ)₂, aryl, C₁₋₃alkylenearyl, heteroaryl, C(═O)ORₑ, C(═O)Rₑ, OC(═O)Rₑ, halo, CN, CF₃, NO₂, N(Rₑ)₂, ORₑ, OC₁₋₆perfluoralkyl, OC(═O)N(Rₑ)₂, C(═O)N(Rₑ)₂, SRₑ, SO₂Rₑ, SO₃Rₑ, oxo(═O), and CHO; and

n is 0 or 1.

Within certain compounds of Formula II, one or more variables are defined as follows:

X is CH₂, CH₂CH₂, CH═CH, CH(CH₃), CH(CH₂CH₃), CH₂CH(CH₃) or C(CH₃)₂;

Y is absent (i.e., represent a direct bond between X and A), S or NH;

A is an aromatic ring or an aromatic bicyclic ring system (i.e., at least one ring is aromatic); in certain embodiments A is imidazolyl, pyrazolyl, 1,2,3-triazolyl, pyridizinyl, pyrimidinyl, pyrazinyl, 1,3,5-triazinyl, purinyl, cinnolinyl, phthalazinyl, quinazolinyl, quinoxalinyl, 1,8-naphthyridinyl, pteridinyl, 1H-indazolyl or benzimidazolyl, each of which is optionally substituted as described above. Preferred A groups include:

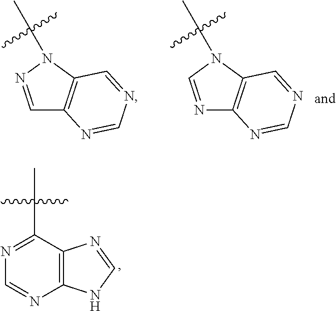

each of which is optionally substituted as described above;

n is 0; in certain embodiments, the ring comprising V, W and Z is

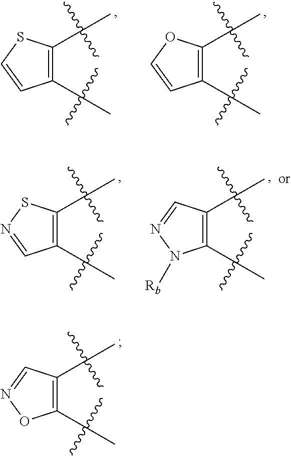

R₁ is C₁-C₆alkyl, phenyl, biphenyl, benzyl, phenethyl, pyridinyl, cyclohexyl, cyclopentyl, piperazinyl, or morpholinyl; each of which is unsubstituted or substituted with from 0 to 3 substituents independently chosen from halo, C₁-C₆alkyl, C₁-C₆alkoxy, (CH₂)₃N(CH₃)₂, C(—O)NH₂, phenyl, NO₂, NH₂, and CO₂H.

Representative compounds of Formula II include, but are not limited to:
6-[1-(6-amino-purin-9-yl)-ethyl]-3-bromo-1-methyl-5-phenyl-1,5-dihydro-pyrazolo[3,4-d]pyrimidin-4-one;
3-bromo-1-methyl-5-phenyl-6-[(1-(9H-purin-6-ylsulfanyl)-ethyl]-1,5-dihydro-pyrazolo[3,4-d]pyrimidin-4-one;
3-methyl-5-phenyl-6-(9H-purin-6-ylsulfanylmethyl)-5H-isoxazolo[5,4-d]pyrimidin-4-one;
6-(6-amino-purin-6-ylmethyl)-3-methyl-5-phenyl-5H-isoxazolo[5,4-d]pyrimidin-4-one;
2-[1-(4-amino-benzoimidazol-1-yl)-ethyl]-3-phenyl-3H-pyrido[3,2-d]pyrimidin-4-one;
   or
3-phenyl-2-[1-(9H-purin-6-ylamino)-ethyl]-3H-pyrido[3,2-d]-pyrimidin-4-one;

or a pharmaceutically acceptable salt and/or hydrate of any of the foregoing compounds.

One selective PIK3CD inhibitor of Formula II has the structure:

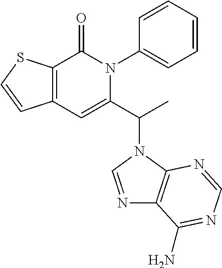

or is a pharmaceutically acceptable salt and/or hydrate thereof.

Compounds of Formulas III and IV, including the pharmaceutically acceptable salts and/or hydrates thereof, are disclosed as selective PIK3CD inhibitors in PCT International Application Publication No. WO 09/053,716, and compound descriptions and methods of preparation therein are hereby incorporated by reference.

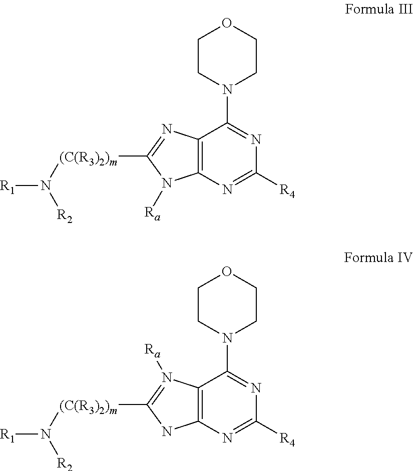

Within Formulas III and IV:

R₁ and R₂ form, together with the N atom to which they are attached:

(a) a 4- to 7-membered saturated N-containing heterocyclic ring which includes 0 or 1 additional heteroatoms selected from N, S and O, the ring being unsubstituted or substituted;

(b) a 4- to 7-membered saturated N-containing heterocyclic ring which includes 0 or 1 additional heteroatoms selected from N, S and O, the ring being fused to a second ring selected from a 4- to 7-membered saturated N-containing heterocyclic ring as defined above, a 5- to 12-membered unsaturated heterocyclic ring, a 5- to 7-membered saturated O-containing heterocyclic ring, a 3- to 12-membered saturated carbocyclic ring and an unsaturated 5- to 12-membered carbocyclic ring to form a heteropolycyclic ring system, the heteropolycyclic ring system being unsubstituted or substituted;

(c) a 4- to 7-membered saturated N-containing heterocyclic ring which includes 0 or 1 additional heteroatoms selected from N, S and O and which further comprises, linking two constituent atoms of the ring, a bridgehead group selected from —(CR′₂)ₙ— and —(CR′₂)ᵣ—O—(CR′₂)ₛ— wherein each R′ is independently H or C₁-C₆alkyl, n is 1, 2 or 3, r is 0 or 1 and s is 0 or 1, the remaining ring positions being unsubstituted or substituted; or

(d) a group of the formula:

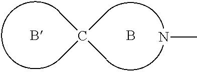

wherein ring B is a 4- to 7-membered saturated N-containing heterocyclic ring which includes 0 or 1 additional heteroatoms selected from N, S and O and ring B′ is a 3- to 12-membered saturated carbocyclic ring, a 5- to 7-membered saturated O-containing heterocyclic ring or a 4- to 7-membered saturated N-containing heterocyclic ring as defined above, each of B and B′ being unsubstituted or substituted;

or one of R₁ and R₂ is C₁-C₆alkyl and the other of R₁ and R₂ is selected from a 3- to 12-membered saturated carbocyclic group which is unsubstituted or substituted, a 5- to 12-membered unsaturated carbocyclic group which is unsubstituted or substituted, a 5- to 12-membered unsaturated heterocyclic group which is unsubstituted or substituted, a 4- to 12-membered saturated heterocyclic group which is unsubstituted or substituted and a C₁-C₆alkyl group which is substituted by a group selected from a 3- to 12-membered saturated carbocyclic group which is unsubstituted or substituted, a 5- to 12-membered unsaturated carbocyclic group which is unsubstituted or substituted, a 5- to 12-membered unsaturated heterocyclic group which is unsubstituted or substituted and a 4- to 12-membered saturated heterocyclic group which is unsubstituted or substituted;

m is 0, 1, or 2;

R₃ is H or C₁-C₆ alkyl;

Rₐ is selected from R, C(O)OR, C(O)NR₂, halo(C₁-C₆)alkyl, SO₂R, or SO₂NR₂, wherein each R is independently H or C₁-C₆ alkyl which is unsubstituted or substituted; and

R₄ is an indole group that is unsubstituted or substituted.

Certain compounds of Formula III or Formula IV further satisfy one or more of the following:

R₄ is optionally substituted indol-4-yl, indol-5-yl, indol-6-yl or indol-7-yl; in certain embodiments the indolyl group is substituted with CN, halo, —C(═O)NH₂, trifluoromethyl, —SO₂CH₃, SO₂N(CH₃)₂ or a 5-membered heteroaryl;

m is 1 or 2 (e.g., 1); and

R₁ and R₂ form a heterocyclic group such as a piperidine, homopiperazine, piperazine, pyrrolidine, azetidine, thiomorpholine or morpholine, each of which is optionally fused to a second ring and each of which is optionally substituted (e.g., with one or more groups independently chosen from C₁-C₆alkyl, C₁-C₆alkoxy, heterocyclyl groups, halo and oxo); certain specific heterocyclic groups formed by R₁ and R₂ include:

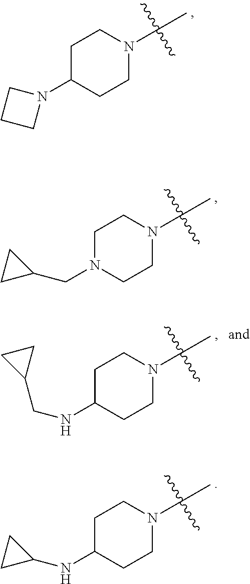

Representative compounds of Formulas III and IV include, but are not limited to:
{1-[9-ethyl-2-(1H-indol-4-yl)-6-morpholin-4-yl-9H-purin-8-ylmethyl]-piperidin-4-yl}-dimethyl-amine;
{1-[9-ethyl-2-(1H-indol-4-yl)-6-morpholin-4-yl-9H-purin-8-ylmethyl]-piperidin-4-yl}-dimethyl-amine;
9-ethyl-2-(5-fluoro-1H-indol-4-yl)-8-[(5)-1-(hexahydro-pyrrolo[1,2-a]pyrazin-2-yl)methyl]-6-morpholin-4-yl-9H-purine;
9-ethyl-8-[(5)-1-(hexahydro-pyrrolo[1,2-a]pyrazin-2-yl)methyl]-2-(1H-indol-4-yl)-6-morpholin-4-yl-9H-purine;
8-(4-azetidin-1-yl-piperidin-1-ylmethyl)-9-ethyl-2-(5-fluoro-1H-indol-4-yl)-6-morpholin-4-yl-9H-purine;
9-ethyl-2-(5-fluoro-1H-indol-4-yl)-6-morpholin-4-yl-8-(4-morpholin-4-yl-piperidin-1-ylmethyl)-9H-purine;
9-ethyl-2-(1H-indol-4-yl)-6-morpholin-4-yl-8-(4-morpholin-4-yl-piperidin-1-ylmethyl)-9H-purine;
2-[9-ethyl-2-(5-fluoro-1H-indol-4-yl)-6-morpholin-4-yl-9H-purin-8-ylmethyl]-1,2,3,4-tetrahydro-isoquinoline;
2-[9-ethyl-2-(1H-indol-4-yl)-6-morpholin-4-yl-9H-purin-8-ylmethyl]-1,2,3,4-tetrahydro-isoquinoline;
2-{4-[9-ethyl-2-(1H-indol-4-yl)-6-morpholin-4-yl-9H-purin-8-ylmethyl]-piperazin-1-yl}-isobutyramide;
8-[4-(3,3-difluoro-azetidin-1-yl)-piperidin-1-ylmethyl]-9-ethyl-2-(5-fluoro-1H-indol-4-yl)-6-morpholin-4-yl-9H-purine;
8-[4-(3,3-difluoro-azetidin-1-yl)-piperidin-1-ylmethyl]-9-ethyl-2-(1H-indol-4-yl)-6-morpholin-4-yl-9H-purine;
2-{4-[9-ethyl-2-(5-fluoro-1H-indol-4-yl)-6-morpholin-4-yl-9H-purin-8-ylmethyl]-2,2-dimethyl-piperazin-1-yl}-acetamide;
2-{4-[9-ethyl-2-(1H-indol-4-yl)-6-morpholin-4-yl-9H-purin-8-ylmethyl]-2,2-dimethyl-piperazin-1-yl}-acetamide;
8-[9-ethyl-2-(5-fluoro-1H-indol-4-yl)-6-morpholin-4-yl-9H-purin-8-ylmethyl]-2,8-diaza-spiro[4.5]decan-3-one;
8-[9-ethyl-2-(1H-indol-4-yl)-6-morpholin-4-yl-9H-purin-8-ylmethyl]-2,8-diaza-Spiro[4.5]decan-3-one;
1-{1-[9-ethyl-2-(1H-indol-4-yl)-6-morpholin-4-yl-9H-purin-8-ylmethyl]-piperidin-4-yl}-azetidin-2-one;
1-{1-[9-ethyl-2-(5-fluoro-1H-indol-4-yl)-6-morpholin-4-yl-9H-purin-8-ylmethyl]-piperidin-4-yl}-azetidin-2-one;
9-ethyl-8-[4-(3-fluoro-azetidin-1-yl)-piperidin-1-ylmethyl]-2-(5-fluoro-1H-indol-4-yl)-6-morpholin-4-yl-9H-purine;
9-ethyl-8-[4-(3-fluoro-azetidin-1-yl)-piperidin-1-ylmethyl]-2-(1H-indol-4-yl)-6-morpholin-4-yl-9H-purine;
9-[9-ethyl-2-(5-fluoro-1H-indol-4-yl)-6-morpholin-4-yl-9H-purin-8-ylmethyl]-1-oxa-4,9-diaza-spiro[5.5]undecan-3-one;
9-[9-ethyl-2-(1H-indol-4-yl)-6-morpholin-4-yl-9H-purin-8-ylmethyl]-1-oxa-4,9-diaza-spiro[5.5]undecan-3-one;
1-[9-ethyl-2-(5-fluoro-1H-indol-4-yl)-6-morpholin-4-yl-9H-purin-8-ylmethyl]-piperidine-4-carboxylic acid amide;
2-{4-[9-ethyl-2-(5-fluoro-1H-indol-4-yl)-6-morpholin-4-yl-9H-purin-8-ylmethyl]-piperazin-1-yl}-isobutyramide;
2-{(cis)-4-[9-ethyl-2-(5-fluoro-1H-indol-4-yl)-6-morpholin-4-yl-9H-purin-8-ylmethyl]-2,6-dimethyl-piperazin-1-yl}-acetamide;
2-{(cis)-4-[9-ethyl-2-(1H-indol-4-yl)-6-morpholin-4-yl-9H-purin-8-ylmethyl]-2,6-dimethyl-piperazin-1-yl}-acetamide;
2-{(5)-4-[9-ethyl-2-(5-fluoro-1H-indol-4-yl)-6-morpholin-4-yl-9H-purin-8-ylmethyl]-2-isopropyl-piperazin-1-yl}-acetamide;
2-{(5)-4-[9-ethyl-2-(1H-indol-4-yl)-6-morpholin-4-yl-9H-purin-8-ylmethyl]-2-isopropyl-piperazin-1-yl}-acetamide;
9-ethyl-2-(1H-indol-4-yl)-6-morpholin-4-yl-8-[4-(tetrahydro-pyran-4-yl)-piperazin-1-ylmethyl]-9H-purine;
4-[9-ethyl-2-(5-fluoro-1H-indol-4-yl)-6-morpholin-4-yl-9H-purin-8-ylmethyl]-6,6-dimethyl-piperazin-2-one;
4-[9-ethyl-2-(1H-indol-4-yl)-6-morpholin-4-yl-9H-purin-8-ylmethyl]-6,6-dimethyl-piperazin-2-one;
8-(2,2-dimethyl-morpholin-4-ylmethyl)-9-ethyl-2-(5-fluoro-1H-indol-4-yl)-6-morpholin-4-yl-9H-purine;
9-ethyl-2-(5-fluoro-1H-indol-4-yl)-6-morpholin-4-yl-8-(3-morpholin-4-yl-azetidin-1-ylmethyl)-9H-purine;
9-ethyl-2-(1H-indol-4-yl)-6-morpholin-4-yl-8-(3-morpholin-4-yl-azetidin-1-ylmethyl)-9H-purine;
9-ethyl-2-(1H-indol-4-yl)-6-morpholin-4-yl-8-[4-(2,2,2-trifluoro-ethyl)-piperazin-1ylmethyl]-9H-purine;
9-ethyl-2-(5-fluoro-1H-indol-4-yl)-6-morpholin-4-yl-8-[4-(2,2,2-trifluoro-ethyl)-piperazin-1-ylmethyl]-9H-purine;
9-ethyl-2-(1H-indol-4-yl)-6-morpholin-4-yl-8-(4-pyrazol-1-yl-piperidin-1-ylmethyl)-9H-purine;
9-ethyl-2-(5-fluoro-1H-indol-4-yl)-6-morpholin-4-yl-8-(4-pyrazol-1-yl-piperidin-1-ylmethyl)-9H-purine;
9-ethyl-2-(5-fluoro-1H-indol-4-yl)-6-morpholin-4-yl-8-[4-(1H-pyrazol-3-yl)-piperidin-1-ylmethyl]-9H-purine;
9-ethyl-2-(1H-indol-4-yl)-6-morpholin-4-yl-8-[4-(1H-pyrazol-3-yl)-piperidin-1-ylmethyl]-9H-purine;
1-[9-ethyl-2-(5-fluoro-1H-indol-4-yl)-6-morpholin-4-yl-9H-purin-8-ylmethyl]-piperidine-4-carboxylic acid;
1-[9-ethyl-2-(5-fluoro-1H-indol-4-yl)-6-morpholin-4-yl-9H-purin-8-ylmethyl]-4-methyl-piperidine-4-carboxylic acid amide;
4-{1-[9-ethyl-2-(5-fluoro-1H-indol-4-yl)-6-morpholin-4-yl-9H-purin-8-ylmethyl]-piperidin-4-yl}-morpholin-3-one;
4-{1-[9-ethyl-2-(1H-indol-4-yl)-6-morpholin-4-yl-9H-purin-8-ylmethyl]-piperidin-4-yl}-morpholin-3-one;
4-[9-ethyl-2-(5-fluoro-1H-indol-4-yl)-6-morpholin-4-yl-9H-purin-8-ylmethyl]-1-isopropyl-piperazin-2-one;
4-[9-ethyl-2-(1H-indol-4-yl)-6-morpholin-4-yl-9H-purin-8-ylmethyl]-1-isopropyl-piperazin-2-one;
9-ethyl-2-(5-fluoro-1H-indol-4-yl)-6-morpholin-4-yl-8-[4-(tetrahydro-pyran-4-yl)-piperazin-1-ylmethyl]-9H-purine;
8-[4-(1,1-dioxo-hexahydro-1-thiopyran-4-yl)-piperazin-1-ylmethyl]-9-ethyl-2-(5-fluoro-1H-indol-4-yl)-6-morpholin-4-yl-9H-purine;
8-[4-(1,1-dioxo-hexahydro-1-thiopyran-4-yl)-piperazin-1-ylmethyl]-9-ethyl-2-(1H-indol-4-yl)-6-morpholin-4-yl-9H-purine;
(R)-8-[9-ethyl-2-(1H-indol-4-yl)-6-morpholin-4-yl-9H-purin-8-ylmethyl]-octahydro-pyrazino[2,1-c][1,4]oxazine;
(R)-8-[9-ethyl-2-(5-fluoro-1H-indol-4-yl)-6-morpholin-4-yl-9H-purin-8-ylmethyl]-octahydro-pyrazino[2,1-c][1,4]oxazine;
(R)-8-[9-ethyl-2-(5-fluoro-1H-indol-4-yl)-6-morpholin-4-yl-9H-purin-8-ylmethyl]-hexahydro-pyrazino[2,1-c][1,4]oxazin-4-one;
8-(2,2-dimethyl-morpholin-4-ylmethyl)-9-ethyl-2-(1H-indol-4-yl)-6-morpholin-4-yl-9H-purine;
8-[4-(1,1-dioxothiomorpholin-4-yl)-piperidin-1-ylmethyl]-9-ethyl-2-(5-fluoro-1H-indol-4-yl)-6-morpholin-4-yl-9H-purine;
8-[4-(1,1-dioxothiomorpholin-4-yl)-piperidin-1-ylmethyl]-9-ethyl-2-(1H-indol-4-yl)-6-morpholin-4-yl-9H-purine;
1-{1-[9-ethyl-2-(1H-indol-4-yl)-6-morpholin-4-yl-9H-purin-8-ylmethyl]-piperidin-4-yl}-pyrrolidin-2-one;

8-[9-ethyl-2-(5-fluoro-1H-indol-4-yl)-6-morpholin-4-yl-9H-purin-8-ylmethyl]-2,8-diaza-spiro[4.5]decan-1-one;
7-[9-ethyl-2-(5-fluoro-1H-indol-4-yl)-6-morpholin-4-yl-9H-purin-8-ylmethyl]-3-oxa-7,9-diaza-bicyclo[3.3.1]nonane;
8-[9-ethyl-2-(1H-indol-4-yl)-6-morpholin-4-yl-9H-purin-8-ylmethyl]-2,8-diaza-spiro[4.5]decan-1-one; 1′-[9-ethyl-2-(5-fluoro-1H-indol-4-yl)-6-morpholin-4-yl-9H-purin-8-ylmethyl]-[1,4′]bipiperidinyl-2-one;
1′-[9-ethyl-2-(1H-indol-4-yl)-6-morpholin-4-yl-9H-purin-8-yldiethyl]-[1,4′]bipiperidinyl-2-one;
1-{1-[9-ethyl-2-(5-fluoro-1H-indol-4-yl)-6-morpholin-4-yl-9H-purin-8-ylmethyl]-piperidin-4-yl}-pyrrolidin-2-one;
2-{1-[9-ethyl-2-(5-fluoro-1H-indol-4-yl)-6-morpholin-4-yl-9H-purin-8-ylmethyl]-azetidin-3-ylamino}-2-methyl-propionamide;
2-{1-[9-ethyl-2-(1H-indol-4-yl)-6-morpholin-4-yl-9H-purin-8-ylmethyl]-azetidin-3-ylamino}-2-methyl-propionamide;
2-{(S)-1-[9-ethyl-2-(5-fluoro-1H-indol-4-yl)-6-morpholin-4-yl-9H-purin-8-ylmethyl]-pyrrolidin-3-ylamino}-2-methyl-propionamide;
2-({1-[9-ethyl-2-(5-fluoro-1H-indol-4-yl)-6-morpholin-4-yl-9H-purin-8-ylmethyl]-azetidin-3-yl}-methyl-amino)-2-methyl-propionamide;
2-{4-[2-(5-Fluoro-1H-indol-4-yl)-9-methyl-6-morpholin-4-yl-9H-purin-8-ylmethyl]-piperazin-1-yl}-isobutyramide;
2-{4-[2-(1H-indol-4-yl)-9-methyl-6-morpholin-4-yl-9H-purin-8-ylmethyl]-piperazin-1-yl}-isobutyramide;
(R)-8-[2-(1H-indol-4-yl)-6-morpholin-4-yl-9H-purin-8-ylmethyl]-octahydro-pyrazino[2,1-c][1,4]oxazine;
2-{4-[2-(5-Fluoro-1H-indol-4-yl)-6-morpholin-4-yl-9H-purin-8-yldiethyl]piperazin-1-yl}-isobutyramide;
2-{4-[2-(1H-indol-4-yl)-6-morpholin-4-yl-9H-purin-8-ylmethyl]-piperazin-1-yl}-isobutyramide;
2-({1-[9-ethyl-2-(5-fluoro-1H-indol-4-yl)-6-morpholin-4-yl-9H-purin-8-ylmethyl]-azetidin-3-yl}-methyl-amino)-2-methyl-propionamide;
2-({1-[9-ethyl-2-(1H-indol-4-yl)-6-morpholin-4-yl-9H-purin-8-ylmethyl]-azetidin-3-yl}-methyl-amino)-2-methyl-propionamide;
2-{4-[2-(5-Fluoro-1H-indol-4-yl)-9-(2-hydroxy-ethyl)-6-morpholin-4-yl-9H-purin-8-ylmethyl]-piperazin-1-yl}-isobutyramide;
{1-[2-(1H-indol-4-yl)-6-morpholin-4-yl-9H-purin-8-ylmethyl]-piperidin-4-yl}-dimethylamine;
{1-[2-(5-Fluoro-1H-indol-4-yl)-6-morpholin-4-yl-9H-purin-8-ylmethyl]-piperidin-4-yl}-dimethylamine
3-{1-[9-ethyl-2-(5-fluoro-1H-indol-4-yl)-6-morpholin-4-yl-9H-purin-8-ylmethyl]-piperidin-4-yl}-oxazolidin-2-one;
3-{1-[9-ethyl-2-(1H-indol-4-yl)-6-morpholin-4-yl-9H-purin-8-ylmethyl]-piperidin-4-yl}-oxazolidin-2-one; 1-[9-ethyl-2-(1H-indol-4-yl)-6-morpholin-4-yl-9H-purin-8-ylmethyl]-4-morpholin-4-yl-piperidine-4-carboxylic acid amide;
1-[9-ethyl-2-(5-fluoro-1H-indol-4-yl)-6-morpholin-4-yl-9H-purin-8-ylmethyl]-4-morpholin-4-yl-piperidine-4-carboxylic acid;
N-{1-[9-ethyl-2-(1H-indol-4-yl)-6-morpholin-4-yl-9H-purin-8-ylmethyl]-piperidin-4-yl}-N-methyl-methanesulfonamide; and
N-{1-[9-ethyl-2-(5-fluoro-1H-indol-4-yl)-6-morpholin-4-yl-9H-purin-8-ylmethyl]-piperidin-4-yl}-N-methyl-methanesulfonamide;

as well as the pharmaceutically acceptable salts and/or hydrates of any the foregoing compounds.

One selective PIK3CD inhibitor of Formula III has the structure:

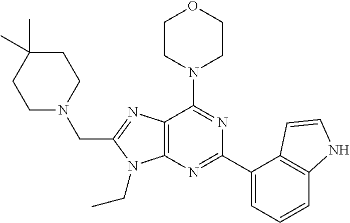

or is a pharmaceutically acceptable salt and/or hydrate thereof.

Compounds of Formula V, including the pharmaceutically acceptable salts and/or hydrates thereof, are disclosed as selective PIK3CD inhibitors in U.S. Patent Application Publication No. 2009/0023761, and PCT International Application Publication No. WO 08/118,455, and compound descriptions and methods of preparation therein are hereby incorporated by reference.

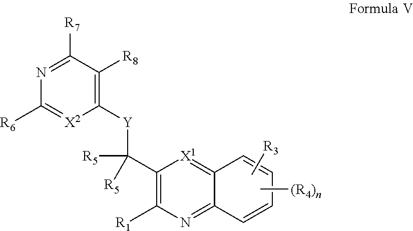

In Formula V:

X¹ is C(R₉) or N;

X² is C(R₁₀) or N;

Y is N(R₁₁), O, or S;

n is 0, 1, 2, or 3;

R₁ is a direct-bonded or oxygen-linked saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0 or 1, R₂ substituents, and the ring is additionally substituted by 0, 1, 2, or 3 substituents independently selected from halo, nitro, cyano, C₁₋₄alkyl, OC₁₋₄alkyl, OC₁₋₄haloalkyl, NHC₁₋₄alkyl, N(C₁₋₄alkyl)C₁₋₄alkyl and C₁₋₄haloalkyl;

R₂ is selected from halo, C₁₋₄haloalkyl, cyano, nitro, —C(═O)Rₐ, C(═O)ORₐ, —C(═O)NRₐRₐ, —C(═NRONRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, NRₐRₐ, N(Rₐ)C(═O)Rₐ, —N(Rₐ)C(═O)ORₐ, —N(Rₐ)C(═O)NRₐRₐ, N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ and —NRₐC₂₋₆alkylORₐ;

or R₂ is selected from C₁₋₆alkyl, phenyl, benzyl, heteroaryl, heterocycle, —(C₁₋₃alkyl)heteroaryl, —(C₁₋₃alkypheterocycle, —O(C₁₋₃alkypheteroaryl, —O(C₁₋₃alkypheterocycle, —NRₐ(C₁₋₃ alkypheteroaryl, —NRₐ(C₁₋₃alkyl)heterocycle, —(C₁₋₃alkyl)phenyl, —O(C₁₋₃allyl)phenyl and —NRₐ(C₁₋₃alkyl)phenyl all of which are substituted by 0, 1, 2 or 3 substituents independently selected from C₁₋₄haloalkyl, OC₁₋₄alkyl, Br, Cl, F, I and C₁₋₄alkyl;

R₃ is selected from H, halo, C₁₋₄haloalkyl, cyano, nitro, —C(═O)Rₐ, —C(═O)ORₐ, C(═O)NRₐRₐ, —C(═NRₐ)NRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, NRₐRₐ, N(Rₐ)C(═O)Rₐ, —N(Rₐ)C(═O)ORₐ, —N(Rₐ)C(═O)NRₐRₐ, N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ, —NRₐC₂₋₆alkylORₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I and C₁₋₆alkyl;

R₄ is, independently, in each instance, halo, nitro, cyano, C₁₋₄alkyl, OC₁₋₄alkyl, OC₁₋₄haloalkyl, NHC₁₋₄alkyl, N(C₁₋₄alkyl)C₁₋₄alkyl or C₁₋₄haloalkyl;

R₅ is, independently, in each instance, H, halo, C₁₋₆alkyl, C₁₋₄haloalkyl, or C₁₋₆alkyl substituted by 1, 2 or 3 substituents selected from halo, cyano, OH, OC₁₋₄alkyl, C₁₋₄alkyl, C₁₋₃haloalkyl, OC₁₋₄alkyl, NH₂, NHC₄alkyl, N(C₁₋₄alkyl)C₁₋₄alkyl;

or both R₅ groups together form a C₃₋₆ spiroalkyl substituted by 0, 1, 2 or 3 substituents selected from halo, cyano, OH, OC₁₋₄ alkyl, C₁₋₄ alkyl, C₁₋₃haloalkyl, OC₁₋₄alkyl, NH₂, NHC₁₋₄alkyl, N(C₁₋₄alkyl)C₁₋₄alkyl;

R₆ is selected from H, C₁₋₆haloalkyl, Br, Cl, F, I, ORₐ, NRₐRₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I and C₁₋₆alkyl;

R₇ is selected from H, C₁₋₆haloalkyl, Br, Cl, F, I, ORₐ, NRₐRₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I and C₁₋₆alkyl;

R₈ is selected from H, halo, C₁₋₄haloalkyl, cyano, nitro, —C(═O)Rₐ, C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, S(═O)Rₐ, S(═O)₂Rₐ, S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, —NRₐRₐ, N(Rₐ)CO)Rₐ, N(Rₐ)C(═O)ORₐ, —N(Rₐ)C(═O)NRₐRₐ, N(Rₐ)C(═NRₐ)NRₐRₐ, N(Rₐ)S(═O)₂Rₐ, —N(Rₐ)S(═O)₂NRₐRₐ, —NRₐC₂₋₆alkylORₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I and C₁₋₆alkyl;

R₉ is selected from H, halo, C₁₋₄haloalkyl, cyano, nitro, —C(═O)Rₐ, —C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, —NRₐRₐ, —N(Rₐ)C(═O)Rₐ, —N(Rₐ)C(═O)ORₐ, —N(Rₐ)C(═O)NRₐRₐ, —N(ROC(═NRONRₐRₐ, —N(Rₐ)S(═O)₂Rₐ, —N(Rₐ)S(═O)₂NRₐRₐ, —NRₐC₂₋₆alkylNRₐRₐ, —NRₐC₂₋₆alkylORₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from halo, C₁₋₄haloalkyl, cyano, nitro, —C(═O)Rₐ, —C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, —NRₐRₐ, —N(Rₐ)C(═O)Rₐ, —N(Rₐ)C(═O)ORₐ, —N(Rₐ)C(═O)NRₐRₐ, —N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ, —NRₐC₂₋₆alkylORₐ;

or R₉ is a saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1, or 2 oxo or thioxo groups, wherein the ring is substituted by 0, 1, 2, 3 or 4 substituents selected from halo, C₁₋₄haloalkyl, cyano, nitro, —C(═O)Rₐ, —C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, NRₐRₐ, N(Rₐ)C(═O)Rₐ, N(Rₐ)C(═O)ORₐ, —N(Rₐ)C(═O)NRₐRₐ, N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ and —NRₐC₂₋₆alkylORₐ;

R₁₀ is H, C₁₋₃alkyl, C₁₋₃haloalkyl, cyano, nitro, CO₂Rₐ, C(═O)NRₐRₐ, —C(═NRₐ)NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, S(═O)R_{b}, S(═O)₂R_{b} or S(═O)₂NRₐRₐ;

R₁₁ is H or C₄alkyl;

Rₐ is independently, at each instance, H or R_{b}; and

R_{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, or —N(C₁₋₄alkyl)C₁₋₄alkyl.

Certain such compounds further satisfy Formula Va:

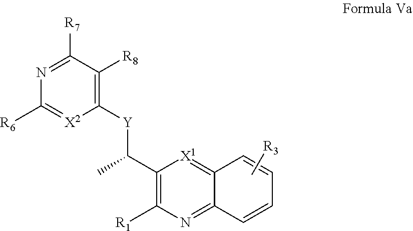

wherein the variables R₁, R₃, R₆, R₇, R₈, X¹, and X² are as described above.

Within certain compounds of Formula V and/or Formula Va, one or more variables satisfy the following:

X₁ is CR₉;

X₂ is N;

R₁ is phenyl or pyridyl; substituted with 0, 1, or 2 substituents independently chosen from C₁-C₄alkyl, halo, C₁-C₄haloalkyl, and C₁-C₄alkOXY;

R₃ is C₁-C₄alkyl, halo, C₁-C₄haloalkyl, or C₁-C₄alkoxy;

R₆, R₇, and R₈ are independently chosen from H, amino, C₁-C₄alkyl, C₁-C₄haloalkyl, and halogen.

Representative compounds of Formula V include, but are not limited to:
5-Chloro-N⁴-((2-(2-chlorophenyl)-8-methylquinolin-3-yl)methyl)pyrimidine-2,4-diamine;
N⁴-((8-Chloro-2-(2-chlorophenyl)quinolin-3-yl)-methyl)pyrimidine-4,6-diamine;
N⁴-((2-(2-chlorophenyl)-8-methylquinolin-3-yl)methyl)-5-(trifluoromethyl)-pyrimidine-2,4-diamine;
N²-((2-(2-chlorophenyl)-8-methylquinolin-3-yl)methyl)-5-(trifluoromethyl)-pyrimidine-2,4-diamine;
6-Chloro-N-((8-chloro-2-phenylquinolin-3-yl)methyl)-5-methoxypyrimidin-4-amine;
5-chloro-N⁴-((S)-1-(8-chloro-2-(pyridin-2-yl)quinolin-3-ypethyl)pyrimidine-2,4-diamine;
4-(8-chloro-2-(2-chlorophenyl)quinoline-3-sulfonamido)picolinamide;
4-((2-(2-chlorophenyl)-8-methylquinolin-3-yl)methylamino)picolinamide; or
N⁴-((8-Chloro-2-(2-chlorophenyl)quinolin-3-yl)methyl)-5-fluoropyrimidine-2,4-diamine;

as well as the pharmaceutically acceptable salts and/or hydrates of any the foregoing compounds.

One selective PIK3CD inhibitor of Formula V has the structure:

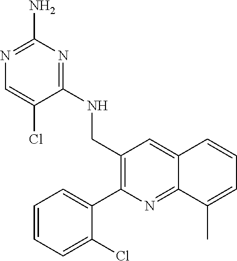

or is a pharmaceutically acceptable salt and/or hydrate thereof.

Compounds of Formula VI are disclosed as selective PIK3CD inhibitors in PCT International Application Publication No. WO 08/000,421, and compound descriptions and methods of preparation therein are hereby incorporated by reference.

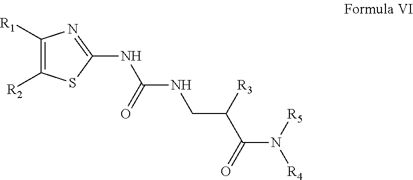

### Within Formula VI:

R₁ is C₁₋₃alkyl;

R₂ is phenyl, naphthyl, or biphenylyl, each being optionally substituted by one or more substituents selected from halogen, SO₂C₁₋₃alkyl, acyl and a 5 or 6 membered heteroaryl; or an optionally substituted 5- or 6-membered heteroaryl;

R₃ is H or C₁₋₃allyl;

R₄ is phenyl, naphthyl or biphenylyl, each being optionally substituted by C₁₋₄alkyl; or an optionally substituted 5- or 6-membered heteroaryl comprising at least one N as heteroatom; provided that R₄ is other than naphthyl when R₂ is phenyl substituted by SO₂C₁₋₃alkyl and optionally halogen; and

R₅ is H or C₁₋₃alkyl.

Certain compounds of Formula VI further satisfy one or more of the following conditions:

R₁ is methyl;

R₃ is H;

R₅ is H;

R₂ is phenyl that is substituted with halogen SO₂C₁₋₃alkyl, C(O)C₁₋₃alkyl, a 5-membered heteroaryl, or a 5- or 6-membered heteroaryl.

Representative compounds of Formula VI include, but are not limited to, compounds that satisfy the formula:

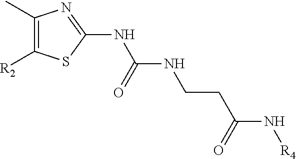

wherein R₂ is selected from:

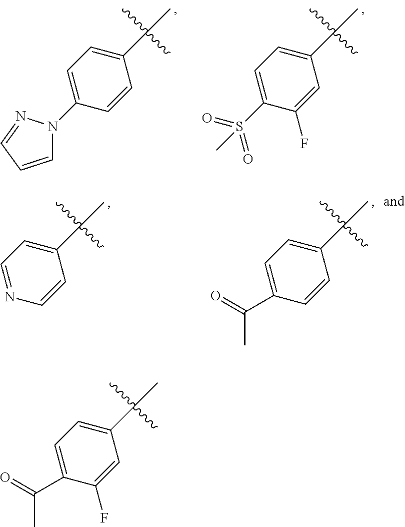

and R₄ is selected from:

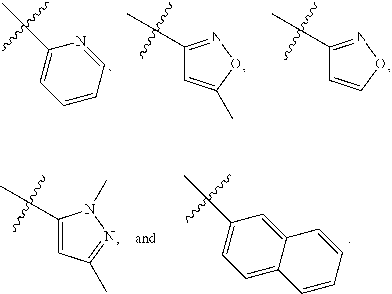

In certain such compounds:

(a) R₂ is

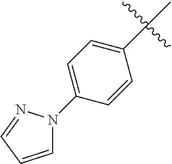

### and R₄ is

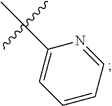

(b) R₂ is

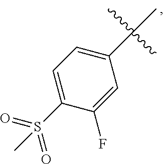

### and R₄ is

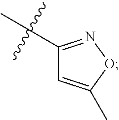

(c) R₂ is

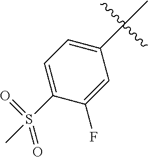

### and R₄ is

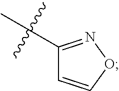

(d) R₂ is

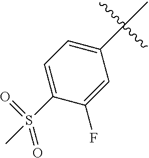

### and R₄ is

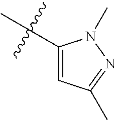

(e) R₂ is

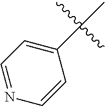

### and R₄ is

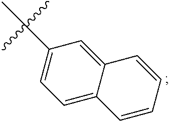

(f) R₂ is

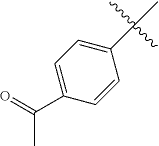

### and R₄ is

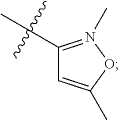

or

(g) R₂ is

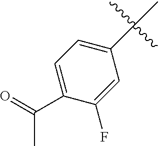

### and R₄ is

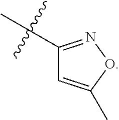

One selective PIK3CD inhibitor of Formula VI has the structure:

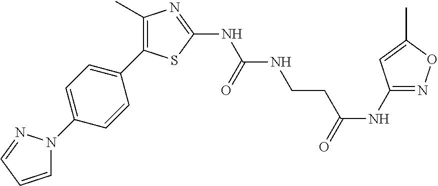

or is a pharmaceutically acceptable salt and/or hydrate thereof.

Compounds of Formula VII, including the pharmaceutically acceptable salts and/or hydrates thereof, are disclosed as selective PIK3CD inhibitors in U.S. Pat. No. 7,585,868, and compound descriptions and methods of preparation therein are hereby incorporated by reference.

### Within Formula VII:

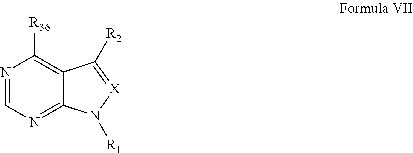

X is N;

R₁ is hydrogen, R₃-substituted or unsubstituted alkyl, unsubstituted cycloalkyl, unsubstituted heterocycloalkyl, or R₃-substituted heteroaryl;

R₂ is R₄-substituted heteroaryl;

R₃ is halogen, —CN, —OR₅, —S(O)ₙR₆, —NR₇R₈, —C(O)R₉, —NR₁₂—C(O)—OR_{D}, —C(O)NR₁₄R₁₅, —NR₁₆S(O)₂R₁₇, R₁₉-substituted or unsubstituted alkyl, R₁₉-substituted or unsubstituted heteroalkyl, R₁₉-substituted or unsubstituted cycloalkyl, R₁₉-substituted or unsubstituted heterocycloalkyl, R₁₉-substituted or unsubstituted aryl, or R₁₉-substituted or unsubstituted heteroaryl, wherein n is an integer from 0 to 2;

R₃₆ is —NR₃₇R₃₈;

R₄ is halogen, —OR₂₀, or —NR₂₂R₂₃;

R₅, R₆, R₇, R₈, R₉, R₁₀, R₁₁, R₁₂, R₁₃, R₁₄, R₁₅, R₁₆, and R₁₇ are independently hydrogen, R₃₅-substituted or unsubstituted alkyl, R₃₅-substituted or unsubstituted heteroalkyl, unsubstituted cycloalkyl, R₃₅-substituted or unsubstituted heterocycloalkyl, R₃₅-substituted or unsubstituted aryl, or R₃₅-substituted or unsubstituted heteroaryl;

R₂₀, R₂₂, and R₂₃ are hydrogen;

R₁₉ and R₃₅ are independently hydrogen, halogen, unsubstituted alkyl, unsubstituted heteroalkyl, unsubstituted cycloalkyl, unsubstituted heterocycloalkyl, unsubstituted aryl, or unsubstituted heteroaryl; and

R₃₇ and R₃₈ are hydrogen.

In certain embodiments, compounds of Formula VII satisfy one or more or the following:

R₃₆ is NH₂;

R₁ is C₁-C₆alkyl or C₃-C₈cycloalkyl;

R₂ is phenyl, naphthyl, pyridinyl, pyrimidinyl, azaindolyl, indolyl, indazolyl, quinazolinyl, pyrazolo[3,4-d]pyrimidinyl, or quinolinyl, each of which is unsubstituted or substituted with from 1 to 3 substituents independently chosen from cyano, halo, hydroxy, C(═O)H, C(═O)NH₂, SO₂NH₂, C₁-C₄alkyl, C₁-C₄alkoxy.

Representative compounds of Formula VII include, but are not limited to
1H-pyrazolo[3,4-d]pyrimidin-4-amine;
3-iodo-1H-pyrazolo[3,4-d]pyrimidin-4-amine;
3-iodo-1-isopropyl-1H-pyrazolo[3,4-d]pyrimidin-4-amine;
3-iodo-1-isopropyl-1H-pyrazolo[3,4-d]pyrimidin-4-amine;
4-(4-amino-1-isopropyl-1H-pyrazolo[3,4-d]pyrimidin-3-yl)-benzenesulfonamide;
1-isopropyl-3-(3-methoxy-4-methylphenyl)-1H-pyrazolo[3,4-d]pyrimidin-4-amine;
6-(4-amino-1-isopropyl-1H-pyrazolo[3,4-d]pyrimidin-3-yl)naphthalen-2-ol;
tert-butyl 4-(4-amino-1-isopropyl-1H-pyrazolo[3,4-d]pyrimidin-3-yl)-2-methoxyphenylcarbamate;
3-(4-amino-3-methoxyphenyl)-1-isopropyl-1H-pyrazolo[3,4-d]pyrimidin-4-amine;
5-(4-amino-1-isopropyl-1H-pyrazolo[3,4-d]pyrimidin-3-yl)pyridine-2-carbonitrile;
3-(3-(benzyloxy)-5-fluorophenyl)-1-isopropyl-1H-pyrazolo[3,4-d]pyrimidin-4-amine;
3-(4-amino-1-isopropyl-1H-pyrazolo[3,4-d]pyrimidin-3-yl)-5-fluorophenol;
1-isopropyl-3-(3,4-dimethoxyphenyl)-1H-pyrazolo[3,4-d]pyrimidin-4-amine;
(3-(4-amino-1-isopropyl-1H-pyrazolo[3,4-d]pyrimidin-3-yl)phenyl)methanol;
3-(4-amino-1-isopropyl-1H-pyrazolo[3,4-d]pyrimidin-3-yl)-N-(4,5-dihydrothiazol-2-yl)benzamide;
1-(4-(4-amino-1-isopropyl-1H-pyrazolo[3,4-d]pyrimidin-3-yl)phenyl)ethanone;
(3-(4-amino-1-isopropyl-1H-pyrazolo[3,4-d]pyrimidin-3-yl)phenyl)methanol;
5-(4-amino-1-isopropyl-1H-pyrazolo[3,4-d]pyrimidin-3-yl)-3-methylthiophene-2-carbaldehyde;
5-(4-amino-1-isopropyl-1H-pyrazolo[3,4-d]pyrimidin-3-yl)furan-3-carbaldehyde;
N-[3-(4-amino-1-isopropyl-1H-pyrazolo[3,4-d]pyrimidin-3-yl)-phenyl]-methanesulfonamide;
3-(4-amino-1-isopropyl-1H-pyrazolo[3,4-d]pyrimidin-3-yl)benzonitrile;
N-[4-(4-amino-1-isopropyl-1H-pyrazolo[3,4-d]pyrimidin-3-yl)-phenyl]-methanesulfonamide;
3-(4-amino-1-isopropyl-1H-pyrazolo[3,4-d]pyrimidin-3-yl)-benzenesulfonamide;
2-(4-amino-1-isopropyl-1H-pyrazolo[3,4-d]pyrimidin-3-yl)benzo[b]thiophene-5-carbaldehyde;
5-(4-amino-1-isopropyl-1H-pyrazolo[3,4-d]pyrimidin-3-yl)-1H-indole-3-carbaldehyde;
3-(benzo[c][1,2,5]oxadiazol-6-yl)-1-isopropyl-1H-pyrazolo[3,4-d]pyrimidin-4-amine;
2-(4-(4-amino-1-isopropyl-1H-pyrazolo[3,4-d]pyrimidin-3-yl)phenyl)acetonitrile;
2-(3-(4-amino-1-isopropyl-1H-pyrazolo[3,4-d]pyrimidin-3-yl)phenyl)acetonitrile;
1-isopropyl-3-(4-methoxyphenyl)-1H-pyrazolo[3,4-d]pyrimidin-4-amine;
1-isopropyl-3-(3-methoxyphenyl)-1H-pyrazolo[3,4-d]pyrimidin-4-amine;
1-isopropyl-3-(pyridin-3-yl)-1H-pyrazolo[3,4-d]pyrimidin-4-amine;
1-isopropyl-3-(pyrimidin-5-yl)-1H-pyrazolo[3,4-d]pyrimidin-4-amine;
3-(2,3-dihydrobenzo[b][1,4]dioxin-6-yl)-1-isopropyl-1H-pyrazolo[3,4-d]pyrimidin-4-amine;
1-(3-(4-amino-1-isopropyl-1H-pyrazolo[3,4-d]pyrimidin-3-yl)phenyl)ethanone; and
4-(4-amino-1-isopropyl-1H-pyrazolo[3,4-d]pyrimidin-3-yl)phenol;

as well as a pharmaceutically acceptable salts and/or hydrates of any of the foregoing compounds.

Compounds of Formula VIII, including the pharmaceutically acceptable salts and/or hydrates thereof, are disclosed as selective PIK3CD inhibitors in US Patent Application Publication No. 2009/0082356, and compound descriptions and methods of preparation therein are hereby incorporated by reference.

In Formula VIII:

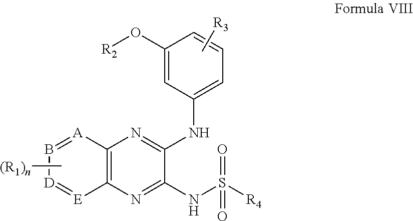

A, B, D and E are independently selected from C and N;

R₁ is selected from H, halogen, nitro, C₁-C₆alkyl, C₂-C₆alkenyl, and C₂-C₆alkynyl;

R₂ is selected from H, C₁-C₆-alkyl, C₂-C₆alkenyl, and C₂-C₆alkynyl;

R₃ is selected from H, halo, C₁-C₆alkyl, C₂-C₆alkenyl, C₂-C₆alkynyl, alkoxy, aryl, and heteroaryl;

R₄ is selected from C₁-C₆alkyl, C₂-C₆alkenyl, C₂-C₆alkynyl, aryl, heteroaryl, C₃-C₈cycloalkyl, heterocycloalkyl, arylC₁-C₆-alkyl, heteroarylC₁-C₆-alkyl, C₃-C₈cycloalkyl C₁-C₆alkyl, heterocycloalkylC₁-C₆alkyl, arylC₂-C₆alkenyl and heteroarylC₂-C₆alkenyl; and

n is an integer selected from 0, 1, 2, 3, and 4.

Within certain embodiments, compounds of Formula VIII further satisfy one or more of the following:

N is 0, 1 or 2 and each R₁ (if present) is independently chosen from halogen;

A, B, D and E are each CH; or no more than one of A, B, D and E is N;

R₂ is C₁-C₄alkyl (e.g., methyl);

R₃ is H, halogen or C₁-C₄alkoxy (e.g., methoxy);

R₄ is phenyl, benzyl, 5- or 6-membered heteroaryl, or (5- or 6-membered heteroaryl)-CH₂—; each of which is unsubstituted or substituted; representative substituents for the phenyl or heteroaryl rings include, but are not limited to, oxo, cyano, halo, COOH, CONH₂, C₁-C₄alkyl, haloC₁-C₄alkyl, aminoC₁-C₄alkyl, hydroxyC₁-C₄alkyl, C₁-C₄alkoxy, 5- or 6-membered heteroaryl, 6-membered heterocycloalkyl, (6-membered heterocycloalkyl)-CH₂—, mono- or di-(C₁-C₄alkyl)amino, NHC(═O)R, C(═O)R, SO₂R, wherein R is H, C₁-C₆alkyl, C₁-C₆alkoxy, or a 5-membered heteroaryl.

Representative compounds of Formula VIII include, but are not limited to
4-cyano-N-{3-[(2,5-dimethoxyphenyl)amino]quinoxalin-2-yl}benzenesulfonamide;
N-{3-[(3,5-dimethoxyphenyl)amino}quinoxalin-2-yl]benzenesulfonamide;
3-[({3-[(3,5-dimethoxyphenyl)amino]quinoxalin-2-yl}amino)sulfonyl]benzoic acid;
N-{3-[(2,5-dimethoxyphenyl)amino]quinoxalin-2-yl}-1-methyl-1H-imidazole-4-sulfonamide;
N-{3-[(2,5-dimethoxyphenyl)amino]quinoxalin-2-yl}-4-methylbenzene sulfonamide;
N-{3-[(3,5-dimethoxyphenyl)amino]quinoxalin-2-yl}-3-methylbenzene sulfonamide;
N-{3-[(2,5-dimethoxyphenyl)amino]quinoxalin-2-yl}-3-methylbenzene sulfonamide;
N-{3-[(3,5-dimethoxyphenyl)amino]quinoxalin-2-yl}-4-methylbenzene sulfonamide;
5-bromo-N-{3-[(3,5-dimethoxyphenyl)amino]quinoxalin-2-yl}thiophene-2-sulfonamide;
N-{3-[(2,5-dimethoxyphenyl)amino]quinoxalin-2-yl}-1-pyridin-3-ylmethane sulfonamide;
Methyl 3-{4-[({3-[(3,5-dimethoxyphenyl)amino]quinoxalin-2-yl}amino)sulfonyl]phenyl}propanoate;
Methyl 4-[({3-[(3,5-dimethoxyphenyl)amino]quinoxalin-2-yl}amino)sulfonyl]benzoate;
N-{3-[(2,5-dimethoxyphenyl)amino]quinoxalin-2-yl}-3-fluorobenzene sulfonamide;
N-{3-[(3,5-dimethoxyphenyl)amino]quinoxalin-2-yl}-4-(methylsulfonyl)benzene sulfonamide;
N-{3-[(2,5-dimethoxyphenyl)amino]quinoxalin-2-yl}-2,3-dihydro-1,4-benzodioxine-6-sulfonamide;
N-{3-[(2,5-dimethoxyphenyl)amino]quinoxalin-2-yl}-4-(pyrrolidin-1-yl-sulfonyl)benzenesulfonamide;
N-{3-[(2,5-dimethoxyphenyl)amino]quinoxalin-2-yl}-3-(methylsulfonyl)benzene sulfonamide;
N-{3-[(2,5-dimethoxyphenyl)amino]quinoxalin-2-yl}-3-(methylsulfonyl)benzene sulfonamide;
N-{3-[(3,5-dimethoxyphenyl)amino]quinoxalin-2-yl}-3-(methylsulfonyl)benzene sulfonamide;
2-cyano-N-{3-[(3,5-dimethoxyphenyl)amino]quinoxalin-2-yl}benzene sulfonamide;
2-cyano-N-{3-[(2,5-dimethoxyphenyl)amino]quinoxalin-2-yl}benzene sulfonamide;
2-chloro-N-{3-[(3,5-dimethoxyphenyl)amino]quinoxalin-2-yl}benzene sulfonamide;
N-{3-[(2,5-dimethoxyphenyl)amino]quinoxalin-2-yl}pyridine-3-sulfonamide;
N-{3-[(3,5-dimethoxyphenyl)amino]quinoxalin-2-yl}-1-methyl-1H-imidazole-4-sulfonamide;
N-{3-[(2,5-dimethoxyphenyl)amino]quinoxalin-2-yl}-4-fluorobenzene sulfonamide;
N-{3-[(2,5-dimethoxyphenyl)amino]pyrido[2,3-b]pyrazin-2-yl}benzene sulfonamide;
N-{3-[(3,5-dimethoxyphenyl)amino]quinoxalin-2-yl}-4-fluorobenzene sulfonamide;
4-cyano-N-{3-[(3,5-dimethoxyphenyl)amino]quinoxalin-2-yl}benzene sulfonamide;
N-{3-[(2,5-dimethoxyphenyl)amino]quinoxalin-2-yl}benzenesulfonamide;
N-{3-[(2,5-dimethoxyphenyl)amino]quinoxalin-2-yl}methanesulfonamide;
N-{3-[(3,5-dimethoxyphenyl)amino]quinoxalin-2-yl}thiophene-3-sulfonamide;
N-{3-[(3,5-dimethoxyphenyl)amino]quinoxalin-2-yl}methanesulfonamide;
3-[({3-[(2,5-dimethoxyphenyl)amino]quinoxalin-2-yl}amino)sulfonyl]benzoic acid;
methyl 4-[({3-[(2,5-dimethoxyphenyl)amino]quinoxalin-2-yl}amino)sulfonyl]benzoate;
methyl 3-[({3-[(3,5-dimethoxyphenyl)amino]quinoxalin-2-yl}amino)sulfonyl]thiophene-2-carboxylate;
5-chloro-N-{3-[(2,5-dimethoxyphenyl)amino]quinoxalin-2-yl}-1,3-dimethyl-1H-pyrazole-4-sulfonamide;
4-chloro-N-{3-[(2,5-dimethoxyphenyl)amino]quinoxalin-2-yl}benzene sulfonamide;
3-[({3-[(2,5-dimethoxyphenyl)amino]quinoxalin-2-yl}amino)sulfonyl]thiophene-2-carboxylic acid;
3-{-4-[({3-[(3,5-dimethoxyphenyl)amino]quinoxalin-2-yl}amino)sulfonyl]phenyl}propanoic acid;
N-{3-[(3,5-dimethoxyphenyl)amino]quinoxalin-2-yl}-3-methyl-2-oxo-2,3-dihydro-1,3-benzothiazole-6-sulfonamide;
N-{3-[(2,5-dimethoxyphenyl)amino]-quinoxalin-2-yl}-2,1,3-benzothiadiazole-4-sulfonamide;
4-chloro-N-{3-[(3,5-dimethoxyphenyl)amino]quinoxalin-2-yl}benzene sulfonamide;
N-{3-[(2,5-dimethoxyphenyl)amino]quinoxalin-2-yl}-3-methyl-2-oxo-2,3-dihydro-1,3-benzothiazole-6-sulfonamide;
4-bromo-N-{3-[(2,5-dimethoxyphenyl)amino]quinoxalin-2-yl}benzene sulfonamide;
N-{3-[(3,5-dimethoxyphenyl)amino]pyrido[2,3-b]pyrazin-2-yl}benzene sulfonamide;
4-bromo-N-{3-[(3,5-dimethoxyphenyl)amino]quinoxalin-2-yl}benzene sulfonamide;
4-acetyl-N-{3-[(3,5-dimethoxyphenyl)amino]quinoxalin-2-yl}benzene sulfonamide;
N-{3-[(3,5-dimethoxyphenyl)amino]quinoxalin-2-yl}propane-1-sulfonamide;
N-{3-[(2,5-dimethoxyphenyl)amino]quinoxalin-2-yl}thiophene-3-sulfonamide;
4-acetyl-N-{3-[(2,5-dimethoxyphenyl)amino]quinoxalin-2-yl}benzene sulfonamide;
N-{3-[(2,5-dimethoxyphenyl)amino]quinoxalin-2-yl}-1,2-dimethyl-1H-imidazole-5-sulfonamide;
N-{3-[(3,5-dimethoxyphenyl)amino]quinoxalin-2-yl}-2,1,3-benzoxadiazole-4-sulfonamide;
3-chloro-N-{3-[(3,5-dimethoxypheny-1)amino]quinoxalin-2-yl}benzene sulfonamide;
3-cyano-N-{3-[(2,5-dimethoxyphenyl)amino]quinoxalin-2-yl}benzene sulfonamide;
N-{3-[({3-[(3,5-dimethoxyphenyl)amino]quinoxalin-2-yl}amino)sulfonyl]phenyl}acetamide;
N-{3-[(2,5-dimethoxyphenyl)amino]quinoxalin-2-yl}propane-1-sulfonamide;
N-{3-[(2,5-dimethoxyphenyl)amino]quinoxalin-2-yl}-4-(trifluoromethyl)benzene sulfonamide;
4-[({3-[(3,5-dimethoxyphenyl)amino]quinoxalin-2-yl}amino)sulfonyl]butanoic acid;
3-chloro-N-{3-[(2,5-dimethoxyphenyl)amino]quinoxalin-2-yl}benzene sulfonamide;
N-{6-chloro-3-[(3,5-dimethoxyphenyl)amino]quinoxalin-2-yl}benzene sulfonamide;
N-{3-[(2,5-dimethoxyphenyl)amino]quinoxalin-2-yl}-1-pyridin-2-ylmethane sulfonamide;
N-{3-[(2,5-dimethoxyphenyl)amino]quinoxalin-2-yl}-4-methoxybenzene sulfonamide;
N-{3-[(3,5-dimethoxyphenyl)amino]pyrido[2,3-b]pyrazin-2-yl}ethane sulfonamide;
N-{3-[(3,5-dimethoxyphenyl)amino]quinoxalin-2-yl}-3-methoxybenzene sulfonamide;
N-{3-[(3,5-dimethoxyphenyl)amino]quinoxalin-2-yl}-1-pyridin-2-ylmethane sulfonamide;
N-{3-[(3,5-dimethoxyphenyl)amino]quinoxalin-2-yl}-1-pyridin-3-ylmethane sulfonamide;
methyl 3-[({3-[(2,5-dimethoxyphenyl)amino]quinoxalin-2-yl}amino)sulfonyl]thiophene-2-carboxylate;
N-{2-[(2,5-dimethoxyphenyl)amino]pyrido[3,4-b]pyrazin-3-yl}benzene sulfonamide;
N-{3-[(3-methoxyphenyl)amino]quinoxalin-2-yl}benzenesulfonamide;
N-{3-[(3-methoxyphenyl)amino]quinoxalin-2-yl}benzenesulfonamide;
4-chloro-N-{3-[(3-methoxyphenyl)amino]quinoxalin-2-yl}benzenesulfonamide;
N-{3-[(2,5-dimethoxyphenyl)amino]quinoxalin-2-yl}-3-methoxybenzene sulfonamide;
4-[({3-[(2,5-dimethoxyphenyl)amino]quinoxalin-2-yl}amino)sulfonyl]butanoic acid;
N-(3-[(3-methoxyphenyl)amino]quinoxalin-2-yl}methanesulfonamide;
N-(3-[(2,5-dimethoxyphenyl)amino]quinoxalin-2-yl}-4-iodobenzene sulfonamide;
4-bromo-N-{3-[(3-methoxyphenyl)amino]quinoxalin-2-yl}benzene sulfonamide;
4-[({3-[(2,5-dimethoxyphenyl)amino]quinoxalin-2-yl}amino)sulfonyl]benzoic acid;
Methyl 4-[({3-[(2,5-dimethoxyphenyl)amino]quinoxalin-2-yl}amino)sulfonyl]butanoate;
4-[({3-[(3,5-dimethoxyphenyl)amino]quinoxalin-2-yl}amino)sulfonyl]benzoic acid;
N-{3-[(3,5-dimethoxyphenyl)amino]quinoxalin-2-yl}-2-fluorobenzene sulfonamide;
N-(3-{[5-methoxy-2-(1H-pyrrol-1-yl)phenyl]amino}quinoxalin-2-yl)benzene sulfonamide;
methyl 3-[({3-[(2,5-dimethoxyphenyl)amino]quinoxalin-2-yl}amino)sulfonyl]benzoate;
N-{3-[(2,5-dimethoxyphenyl)amino]quinoxalin-2-yl}-6-morpholin-4-yl pyridine-3-sulfonamide;
4-methoxy-N-{3-[(3-methoxyphenyl)amino]quinoxalin-2-yl}benzene sulfonamide;
methyl 3-[({3-[(3,5-dimethoxyphenyl)amino]quinoxalin-2-yl}amino)sulfonyl]benzoate;
3-[({3-[(3,5-dimethoxyphenyl)amino]quinoxalin-2-yl}amino)sulfonyl]thiophene-2-carboxylic acid;
N-{3-[(2-chloro-5-methoxyphenyl)amino]quinoxalin-2-yl}benzenesulfona-mide;
N-{3-[(3,5-dimethoxyphenyl)amino]quinoxalin-2-yl}-2-(methylsulfonyl)benzene sulfonamide;
N-{3-[(2,5-dimethoxyphenyl)amino]quinoxalin-2-yl}-2-fluorobenzene sulfonamide;
4,5-dichloro-N-{3-[(2,5-dimethoxyphenyl)amino]quinoxalin-2-yl}thiophene-2-sulfonamide;
N-{3-[(5-methoxy-2-methylphenyl)amino]quinoxalin-2-yl}benzene sulfonamide;
N-{3-[(3,5-dimethoxyphenyl)amino]quinoxalin-2-yl}-3-fluorobenzene sulfonamide;
N-{3-[(2,5-dimethoxyphenyl)amino]quinoxalin-2-yl}-2-(methylsulfonyl)benzenesulfonamide;
N-{3-[(2,3-dihydro-1,4-benzodioxin-5-ylmethyl)amino]quinoxalin-2-yl}benzenesulfonamide;
N-{3-[(3,5-dimethoxyphenyl)amino]-6-nitroquinoxalin-2-yl}benz-ene sulfonamide;
N-{3-[(3,5-dimethoxyphenyl)amino]quinoxalin-2-yl}-4-(pyrrolidin-1-ylsulfonyl)benzenesulfonamide;
methyl 4-[({3-[(3,5-dimethoxyphenyl)amino]quinoxalin-2-yl}amino)sulfonyl]butanoate;
methyl 5-[({3-[(2,5-dimethoxyphenyl)amino]quinoxalin-2-yl}amino)sulfonyl]-4-methylthiophene-2-carboxylate;
methyl 5-[({3-[(2,5-dimethoxyphenyl)amino]quinoxalin-2-yl}amino)sulfonyl]-1-methyl-1H-pyrrole-2-carboxylate;
methyl 5-[({3-[(3,5-dimethoxyphenyl)amino]quinoxalin-2-yl}amino)sulfonyl]-1-methyl-1H-pyrrole-2-carboxylate;
N-{3-[(2,5-dimethoxyphenyl)amino]quinoxalin-2-yl}thiophene-2-sulfonamide;
2-chloro-N-{3-[(2,5-dimethoxyphenyl)amino]quinoxalin-2-yl}-4-fluorobenzene sulfonamide;
2-chloro-N-{3-[(3,5-dimethoxyphenyl)amino]quinoxalin-2-yl}-4-fluorobenzene sulfonamide;
N-{3-[(3,5-dimethoxyphenyl)amino]quinoxalin-2-yl}pyridine-3-sulfonamide;
3-cyano-N-{3-[(3,5-dimethoxyphenyl)amino]quinoxalin-2-yl}-4-fluorobenzene sulfonamide;
3-cyano-N-{3-[(2,5-dimethoxyphenyl)amino]quinoxalin-2-yl}-4-fluorobenzene sulfonamide;
6-chloro-N-{3-[(3,5-dimethoxyphenyl)amino]quinoxalin-2-yl}pyridine-3-sulfonamide;
N-{3-[(3,5-dimethoxyphenyl)amino]quinoxalin-2-yl}-6-(dimethylamino)pyridine-3-sulfonamide;
N-{3-[(3,5-dimethoxyphenyl)amino]quinoxalin-2-yl}-6-[(3-methoxypropyl)amino]pyridine-3-sulfonamide;
N-{3-[(5-methoxy-2-methylphenyl)amino]quinoxalin-2-yl}pyridine-3-sulfonamide;
N-{3-[(2-chloro-5-methoxyphenyl)amino]quinoxalin-2-yl}-4-cyano benzenesulfonamide;
N-{3-[(2-chloro-5-methoxyphenyl)amino]quinoxalin-2-yl}pyridine-3-sulfonamide;
N-{3-[(3,5-dimethoxyphenyl)amino]quinoxalin-2-yl}-6-methoxypyridine-3-sulfonamide;
N-{3-[(3,5-dimethoxyphenyl)amino]quinoxalin-2-yl}-6-oxo-1,6-dihydropyridine-3-sulfonamide;
N-{3-[(3,5-dimethoxyphenyl)amino]quinoxalin-2-yl}-6-methylpyridine-3-sulfonamide;
N-{3-[(3,5-dimethoxyphenyl)amino]quinoxalin-2-yl}-4-fluoro-2-methylbenzene sulfonamide;
N-{3-[(2,5-dimethoxyphenyl)amino]quinoxalin-2-yl}-6-methylpyridine-3-sulfonamide;
4-cyano-N-{3-[(5-methoxy-2-methylphenyl)amino]quinoxalin-2-yl}benzene sulfonamide;
N-{3-[(5-methoxy-2-methylphenyl)amino]quinoxalin-2-yl}-6-methylpyridine-3-sulfonamide;
N-{3-[(2-chloro-5-methoxyphenyl)amino]quinoxalin-2-yl}-6-methylpyridine-3-sulfonamide;
methyl 5-[({3-[(3,5-dimethoxyphenyl)amino]quinoxalin-2-yl}amino)sulfonyl]pyridine-2-carboxylate;
N-{3-[(2-bromo-5-methoxyphenyl)amino]quinoxalin-2-yl}-1-methyl-1H-imidazole-4-sulfonamide;
N-{3-[(3,5-dimethoxyphenyl)amino]quinoxalin-2-yl}-3-(morpholin-4-ylcarbonyl)benzenesulfonamide;
5-[({3-[(2,5-dimethoxyphenyl)amino]quinoxalin-2-yl}amino)sulfonyl]-4-methyl thiophene-2-carboxylic acid;
5-[({3-[(3,5-dimethoxyphenyl)amino]quinoxalin-2-yl}amino)sulfonyl]-4-methyl thiophene-2-carboxylic acid;
5-[({3-[(2,5-dimethoxyphenyl)amino]quinoxalin-2-yl}amino)sulfonyl]-1-methyl-1H-pyrrole-2-carboxylic acid;
5-[({3-[(3,5-dimethoxyphenyl)amino]quinoxalin-2-yl}amino)sulfonyl]-1-methyl-1H-pyrrole-2-carboxylic acid;
5-[({3-[(3,5-dimethoxyphenyl)amino]quinoxalin-2-yl}amino)sulfonyl]pyridine-2-carboxylic acid;
N-{3-[(3,5-dimethoxyphenyl)amino]quinoxalin-2-yl}-3-(morpholin-4-ylmethyl)benzene sulfonamide;
N-{3-[(3,5-dimethoxyphenyl)amino]quinoxalin-2-yl}-4-[(4-methylpiperazin-1-yl)methyl]benzenesulfonamide;
4-(aminomethyl)-N-{3-[(2,5-dimethoxyphenyl)amino]quinoxalin-2-yl}benzene sulfonamide;
N-{3-[(3,5-dimethoxyphenyl)amino]quinoxalin-2-yl}-3-(hydroxymethyl)benzenesulfonamide;
3-(aminomethyl)-N-{3-[(3,5-dimethoxyphenyl)amino quinoxalin-2-yl}benzenesulfonamide;
N-{3-[(3,5-dimethoxyphenyl)amino]quinoxalin-2-yl}-4-(hydroxymethyl)benzenesulfonamide;
N-{3-[(3,5-dimethoxyphenyl-)amino]quinoxalin-2-yl}-6-(hydroxymethyl)pyridino-3-sulfonamide;
N-{3-[(3,5-dimethoxyphenyl)amino]quinoxalin-2-yl}-4-(morpholin-4-ylmethyl}benzenesulfonamide;
N-{3-[(3,5-dimethoxyphenyl)amino]quinoxalin-2-yl}-3-[(4-methylpiperazin-1-yl)methyl[benzenesulfonamide;
N-{3-[(3,5-dimethoxyphenyl)amino]quinoxalin-2-yl}-4-[(dimethylamino)methyl]benzenesulfonamide;
N-{3-[(3,5-dimethoxyphenyl)amino]quinoxalin-2-yl}-3-[(dimethylamino)methyl]benzenesulfonamide;
4-[({3-[(3,5-dimethoxyphenyl)amino]quinoxalin-2-yl}amino)sulfonyl]benzamide;
4-[({3-[(5-methoxy-2-methylphenyl)amino]quinoxalin-2-yl}amino)sulfonyl]benzamide;
4-[({3-[(3,5-dimethoxyphenyl)amino]quinoxalin-2-yl}amino)sulfonyl]-N-(3-methoxypropyl)benzamide;
4-[({3-[(3,5-dimethoxyphenyl)amino]quinoxalin-2-yl}amino)sulfonyl]-N-[3-(dimethylamino)propyl]benzamide;
3-[({3-[(3,5-dimethoxyphenyl)amino]quinoxalin-2-yl}amino)sulfonyl]-N-[3-(dimethylamino)propyl]benzamide;
5-[({3-[(3,5-dimethoxyphenyl)amino]quinoxalin-2-yl}amino)sulfonyl]-N,N-dimethylpyridine-2-carboxamide;
N-{3-[(3,5-dimethoxyphenyl)amino]quinoxalin-2-yl}-3-[(4-methylpiperazin-1-yl)carbonyl]benzenesulfonamide;
N-{3-[(3,5-dimethoxyphenyl)amino]quinoxalin-2-yl}-6-(morpholin-4-ylcarbonyl)pyridine-3-sulfonamide;
N-{3-[(3,5-dimethoxyphenyl)amino]pyrido[2,3-b]pyrazin-2-yl}ethane sulfonamide;
N-{3-[(3,5-dimethoxyphenyl)amino]quinoxalin-2-yl}-6-[(4-methylpiperazin-1-yl)methyl]pyridine-3-sulfonamide; or
5-(aminomethyl)-N-{3-[(3,5-dimethoxyphenyl)amino]quinoxalin-2-yl}thiophene-2-sulfonamide;

as well as a pharmaceutically acceptable salts and/or hydrates of any of the foregoing compounds.

Compounds of Formula IX and Formula X, including the pharmaceutically acceptable salts and/or hydrates thereof, are disclosed as selective PIK3CD inhibitors in PCT International Patent Application Publication No. WO 07/122,410, and compound descriptions and methods of preparation therein are hereby incorporated by reference.

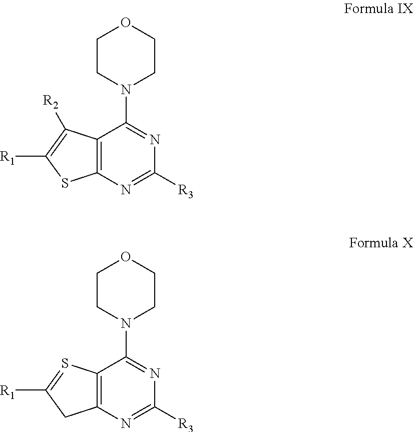

### Within Formulas IX and X:

R₁ is —CH₂N(R₄)(R₅);

R₂ is H, halo or C₁-C₆alkyl;

R₃ is an indole group that is unsubstituted or substituted;

R₄ and R₅ form, together with the N atom to which they are attached, a group selected from piperazine, piperidine and pyrrolidine, which group is unsubstituted or substituted by one or more groups selected from C₁-C₆alkyl, —S(O)₂R₁₀, —S(O)₂—

(alk)_{q}-NR₁₁R₁₂, oxo (=0), -alk-OR₁₀, -(alk)_{q}-Het, a heterocyclyl group and —NR₁₃R₁₄; or one of R₄ and R₅ is C₁-C₆=alkyl and the other is a piperazine, piperidine or pyrrolidine group, which group is unsubstituted or substituted;

R₁₀ is H or C₁-C₆ alkyl which is unsubstituted;
R₁₁ and R₁₂ are each independently selected from H and C₁-C₆alkyl, or R₁₁ and R₁₂ together form, with the N atom to which they are attached, a 5- or 6-membered saturated heterocyclic group;

R₁₃ and R₁₄ are each independently selected from C₁-C₆alkyl, —S(O)₂R₁₀, alk-OR₁₀, -(alk)_{q}-Ph and -(alk)_{q}-Het;

Ph is phenyl;

q is 0 or 1;

Het is a thiazole, imidazole, pyrrole, pyridine or pyrimidine group, which group is unsubstituted or substituted; and

alk is C₁-C₆alkylene.

Within certain compounds of Formula IX and Formula X, one or more of the following criteria are satisfied:

R₂ is H;

R₃ is an indole group that is unsubstituted or substituted with one or two substituents independently chosen from cyano, halo, CONH₂, SO₂CH₃, SO₂N(CH₃)₂, C₁-C₄alkyl, and C₁-C₄haloalkyl;

R₄ and R₅ form, together with the N atom to which they are attached, a group selected from piperazine, piperidine and pyrrolidine, which group is unsubstituted or substituted by one or more groups selected from C₁-C₆alkyl, —S(O)₂R₁₀, —S(O)₂-(alk)_{q}-NR₁₁R₁₂, oxo (=0), -alk-OR₁₀, -(alk)_{q}-Het, a heterocyclyl group and —NR₁₃R₁₄.

Representative compounds of Formulas IX and X include, but are not limited to:
2-(1H-indol-4-yl)-4-morpholin-4-yl-6-[4-(3-morpholin-4-yl-propane-1-sulfonyl)-piperazin-1-ylmethyl]-thieno[3,2-d]pyrimidine;
(3-{4-[2-(1H-indol-4-yl)-4-morpholin-4-yl-thieno[3,2-d]pyrimidin-6-ylmethyl]-piperazine-1-sulfonyl}-propyl)-dimethylamine;
2-(1H-indol-4-yl)-6-(4-methyl-piperazin-1-ylmethyl)-4-morpholin-4-yl-thieno[2,3-d]pyrimidine;
2-(1H-indol-4-yl)-6-(4-methanesulfonyl-piperazin-1-ylmethyl)-4-morpholin-4-yl-thieno[2,3-d]pyrimidine;
2-(7-methyl-1H-indol-4-yl)-6-(4-methyl-piperazin-1-ylmethyl)-4-morpholin-4-yl-thieno[3,2-d]pyrimidine;
2-(1H-indol-4-yl)-7-methyl-6-(4-methyl-piperazin-1-ylmethyl)-4-morpholin-4-yl-thieno[3,2-d]pyrimidine;
benzyl-{1-[2-(1H-indol-4-yl)-4-morpholin-4-yl-thieno[3,2-d]pyrimidine-6-ylmethyl]-piperidin-4-yl}-(2-methoxy-ethyl).-amine;
2-(6-methoxy-1H-indol-4-yl)-6-(4-methyl-piperazin-1-ylmethyl)-4-morpholin-4-yl-thieno[3,2-d]pyrimidine;
1-(2-hydroxyethyl)-4-[2-(1H-indol-4-yl)-4-morpholin-4-yl-thieno[3,2-d]pyrimidine-6-ylmethyl]-piperazin-2-one;
2-(1H-indol-4-yl)-4-morpholin-4-yl-6-(4-thiazol-4-ylmethyl-piperazin-1-ylmethyl)-thieno[3,2-d]pyrimidine;
6-[4-(1H-imidazol-2-ylmethyl)-piperazin-1-ylmethyl]-2-(1H-indol-4-yl)-4-morpholin-4-yl-thieno[3,2-d]pyrimidine;
2-(1H-indol-4-yl)-4-morpholin-4-yl-6-(4-pyridin-2-ylmethyl-piperidin-1-ylmethyl)-thieno[3,2-d]pyrimidine;
2-(1H-indol-4-yl)-4-morpholin-4-yl-6-(4-pyrimidin-2-yl-piperazin-1-ylmethyl)-thieno[3,2-d]pyrimidine;
1′-[2-(1H-indol-4-yl)-4-morpholin-4-yl-thieno[3,2-d]pyrimidin-6-ylmethyl]-[1,4]bipiperidinyl;
2-(1H-indol-4-yl)-6-[4-(1-methyl-1H-imidazol-2-ylmethyl)-piperazin-1-ylmethyl]-4-morpholin-4-yl-thieno[3,2-d]pyrimidine;
[2-(1H-indol-4-yl)-4-morpholin-4-yl-thieno[3,2-d]pyrimidin-6-ylmethyl]-(1-methanesulphonyl-piperidin-4-yl)-methyl-amine;
N-{1-[2-(1H-indol-4-yl)-4-morpholin-4-yl-thieno[3,2-d]pyrimidin-6-ylmethyl]-pyrrolidin-3-yl}-N-methyl-methanesulfonamide;
{1-[2-(1H-indol-4-yl)-4-morpholin-4-yl-thieno[3,2-d]pyrimidin-6-ylmethyl]piperidin-4-yl}-(2-methoxy-ethyl)-thiazol-2-ylmethyl-amine;
N-{1-[2-(1H-indol-4-yl)-4-morpholin-4-yl-thieno[3,2-d]pyrimidin-6-ylmethyl]-pyrrolidin-2-ylmethyl}-N-methyl-methanesulfonamide;
2-(2-methyl-1H-indol-4-yl)-6-(4-methyl-piperazin-1-ylmethyl)-4-morpholin-4-yl-thieno[3,2-d]pyrimidine;
2-(6-fluoro-1H-indol-4-yl)-6-(4-methyl-piperazin-1-ylmethyl)-4-morpholin-4-yl-thieno[3,2-d]pyrimidine;
4-[6-(4-Methyl-piperazin-1-ylmethyl)-4-morpholin-4-yl-thieno[3,2-d]pyrimidin-2-yl]-1H-indole-6-carbonitrile;
[2-(1H-indol-4-yl)-4-morpholin-4-yl-thieno[3,2-d]pyrimidin-6-ylmethyl]-(1-methanesulfonyl-pyrrolidin-3-yl)-methyl-amine;
4-(4-morpholin-4-yl-6-piperazin-1-ylmethyl-thieno[3,2-d]pyrimidin-2-yl)-1H-indole-6-sulfonic acid dimethylamide;
4-[6-(4-cyclopropylmethyl-piperazin-1-ylmethyl)-4-morpholin-4-yl-thieno[3,2-d]pyrimidin-2-yl]-1H-indole-6-sulfonic acid dimethylamide;
2-{4-[2-(6-dimethylsulfamoyl-1H-indol-4-yl)-4-morpholin-4-yl-thieno[3,2-d]pyrimidin-6-ylmethyl]-piperazin-1-yl}-isobutyramide;
4-{4-morpholin-4-yl-6-[4-(2,2,2-trifluoroethyl)-piperazin-1-ylmethyl]-thieno[3,2-d]pyrimidin-2-yl}-1H-indole-6-sulfonic acid dimethylamide;
4-morpholin-4-yl-6-piperazin-1-ylmethyl-2-(6-trifluoromethyl-1H-indol-4-yl)-thieno[3,2-d]pyrimidine;
6-(4-cyclopropylmethyl-piperazin-1-ylmethyl)-4-morpholin-4-yl-2-(6-trifluoromethyl-1H-indol-4-yl)-thieno[3,2-d]pyrimidine;
2-{4-[4-morpholin-4-yl-2-(6-trifluoromethyl-1H-indol-4-yl)-thieno[3,2-d]pyrimidin-6-ylmethyl]-piperazin-1-yl}-isobutyramide;
4-morpholin-4-yl-6-[4-(2,2,2-trifluoro-ethyl)-piperazin-1-ylmethyl]-2-(6-trifluoromethyl-1H-indol-4-yl)-thieno[3,2-d]pyrimidine;
4-morpholin-4-yl-6-piperazin-1-ylmethyl-2-(2-trifluoromethyl-1H-indol-4-yl)-thieno[3,2-d]pyrimidine;
2-{4-[4-morpholin-4-yl-2-(2-trifluoromethyl-1H-indol-4-yl)-thieno[3,2-d]pyrimidin-6-ylmethyl]-piperazin-1-yl}-isobutyramide;
6-(4-cyclopropylmethyl-piperazin-1-ylmethyl)-4-morpholin-4-yl-2-(2-trifluoromethyl-1H-indol-4-yl)-thieno[3,2-d]pyrimidine;
4-morpholin-4-yl-6-[4-(2,2,2-trifluoroethyl)-piperazin-1-ylmethyl]-2-(2-trifluoromethyl-1H-indol-4-yl)-thieno[3,2-d]pyrimidine;
2-(6-methanesulfonyl-1H-indol-4-yl)-4-morpholin-4-yl-6-piperazin-1-ylmethyl-thieno[3,2-d]pyrimidine;
6-(4-cyclopropylmethyl-piperazin-1-ylmethyl)-2-(6-methanesulfonyl-1H-indol-4-yl)-4-morpholin-4-yl-thieno[3,2-d]pyrimidine;
2-{4-[2-(6-methanesulfonyl-1H-indol-4-yl)-4-morpholin-4-yl-thieno[3,2-d]pyrimidin-6-ylmethyl]-piperazin-1-yl}-isobutyramide;
2-(6-methanesulfonyl-1H-indol-4-yl)-4-morpholin-4-yl-6-[4-(2,2,2-trifluoroethyl)piperazin-1-ylmethyl]-thieno[3,2-d]pyrimidine;
2-{4-[2-(5-fluoro-1H-indol-4-yl)-4-morpholin-4-yl-thieno[3,2-d]pyrimidin-6-ylmethyl]-piperazin-1-yl}-isobutyramide;
2-(5-fluoro-1H-indol-4-yl)-4-morpholin-4-yl-6-[4-(2,2,2-trifluoroethyl)-piperazin-1-ylmethyl]-thieno[3,2-d]pyrimidine;
6-(4-cyclopropylmethyl-piperazin-1-ylmethyl)-2-(5-fluoro-1H-indol-4-yl)-4-morpholin-4-yl-thieno[3,2-d]pyrimidine;
4-(4-morpholin-4-yl-6-piperazin-1-ylmethyl-thieno[3,2-d]pyrimidin-2-yl)-1H-indole-6-carboxylic acid amide;
4-{6-[4-(1-carbamoyl-1-methyl-ethyl)-piperazin-1-ylmethyl]-4-morpholin-4-yl-thieno[3,2-d]pyrimidin-2-yl}-1H-indole-6-carboxylic acid amide;
4-{4-morpholin-4-yl-6-[4-(2,2,2-trifluoro-ethyl)-piperazin-1-ylmethyl]-thieno[3,2-d]pyrimidin-2-yl}-1H-indole-6-carboxylic acid amide;
4-[6-(4-cyclopropylmethyl-piperazin-1-ylmethyl)-4-morpholin-4-yl-thieno[3,2-d]pyrimidin-2-yl]-1H-indole-6-carboxylic acid amide;
4-{4-morpholin-4-yl-6-[4-(2,2,2-trifluoroethyl)-piperazin-1-ylmethyl]-thieno[3,2-d]pyrimidin-2-yl}-1H-indole-2-carbonitrile;
2-{4-[2-(2-cyano-1H-indol-4-yl)-4-morpholin-4-yl-thieno[3,2-d]pyrimidin-6-ylmethyl]-piperazin-1-yl}-isobutyramide;
4-[6-(4-cyclopropylmethyl-piperazin-1-ylmethyl)-4-morpholin-4-yl-thieno[3,2-d]pyrimidin-2-yl]-1H-indole-2-carbonitrile;
4-{4-morpholin-4-yl-6-[4-(2,2,2-trifluoroethyl)-piperazin-1-ylmethyl]-thieno[3,2-d]pyrimidin-2-yl}-1H-indole-6-carbonitrile;
4-[6-(4-cyclopropylmethyl-piperazin-1-ylmethyl)-4-morpholin-4-yl-thieno[3,2-d]pyrimidin-2-yl]-1H-indole-6-carbonitrile;
6-(4-cyclopropylmethyl-piperazin-1-ylmethyl)-2-(6-fluoro-1H-indol-4-yl)-4-morpholin-4-yl-thieno[3,2-d]pyrimidine;
2-(6-fluoro-1H-indol-4-yl)-4-morpholin-4-yl-6-[4-(2,2,2-trifluoroethyl)-piperazin-1-ylmethyl]-thieno[3,2-d]pyrimidine;
2-(6-fluoro-1H-indol-4-yl)-4-morpholin-4-yl-6-piperazin-1-ylmethyl-thieno[3,2-d]pyrimidine;
2-(5-fluoro-1H-indol-4-yl)-4-morpholin-4-yl-6-piperazin-1-ylmethyl-thieno[3,2-d]pyrimidine;
4-(4-morpholin-4-yl-6-piperazin-1-ylmethyl-thieno[3,2-d]pyrimidin-2-yl)-1H-indole-6-carbonitrile;
4-[6-(4-isopropyl-piperazin-1-yhriethyl)-4-moipholin-4-yl-thieno[3,2-d]pyrimidin-2-yl]-1H-indole-6-carbonitrile;
2-(6-methanesulfonyl-1H-indol-4-yl)-4-morpholin-4-yl-6-piperidin-1-ylmethyl-thieno[3,2-d]pyrimidine;
2-(6-methanesulfonyl-1H-indol-4-yl)-6-[4-(2-methoxyethyl)-piperidin-1-ylmethyl]-4-morpholin-4-yl-thieno[3,2-d]pyrimidine;
4-{6-[4-(2-methoxy-ethyl)-piperidin-1-ylmethyl]-4-morpholin-4-yl-thieno[3,2-d]pyrimidin-2-yl}-1H-indole-6-carbonitrile;
4-{6-[4-(2-methoxy-ethyl)-piperidin-1-ylmethyl]-4-morpholin-4-yl-thieno[3,2-d]pyrimidin-2-yl}-1H-indole-6-sulfonic acid dimethylamide;
2-(6-methanesulfonyl-1H-indol-4-yl)-4-morpholin-4-yl-6-piperazin-1-ylmethyl-thieno[2,3-d]pyrimidine;
2-(5-fluoro-1H-indol-4-yl)-6-[4-(2-methoxy-ethyl)-piperidin-1-ylmethyl]-4-morpholin-4-yl-thieno[3,2-d]pyrimidine;
4-{6-[4-(2-methoxy-ethyl)-piperidin-1-ylmethyl]-4-morpholin-4-yl-thieno[3,2-d]pyrimidin-2-yl}-1H-indole-2-carbonitrile;
4-[6-(4-methyl-piperazin-1-ylmethyl)-4-morpholin-4-yl-thieno[3,2-d]pyrimidin-2-yl]-1H-indole-6-carboxylic acid dimethylamide;
2-{4-[2-(6-cyano-1H-indol-4-yl)-4-morpholin-4-yl-thieno[3,2-d]pyrimidin-6-ylmethyl]-piperazin-1-yl}-isobutyramide;
2-{4-[2-(6-fluoro-1H-indol-4-yl)-4-morpholin-4-yl-thieno[3,2-d]pyrimidin-6-ylmethyl]-piperazin-1-yl}-isobutyramide;
2-(6-fluoro-1H-indol-4-yl)-4-morpholin-4-yl-6-piperidin-1-ylmethyl-thieno[3,2-d]pyrimidine;
2-(6-fluoro-1H-indol-4-yl)-6-[4-(2-methoxyethyl)-piperidin-1-ylmethyl]-4-morpholin-4-yl-thieno[3,2-d]pyrimidine;
4-(4-morpholin-4-yl-6-piperidin-1-ylmethyl-thieno[3,2-d]pyrimidin-2-yl)-1H-indole-6-carbonitrile;
2-(6-fluoro-1H-indol-4-yl)-4-morpholin-4-yl-6-piperazin-1-ylmethyl-thieno[2,3-d]pyrimidine;
2-{4-[2-(6-fluoro-1H-indol-4-yl)-4-morpholin-4-yl-thieno[3,2-d]pyrimidin-6-ylmethyl]-piperazin-1-yl}-N-methyl-isobutyramide;
2-(6-fluoro-1H-indol-4-yl)-6-[4-(2-methoxyethyl)-piperidin-1-ylmethyl]-7-methyl-4-morpholin-4-yl-thieno[3,2-d]pyrimidine;
4-{6-[4-(2-methoxy-ethyl)-piperidin-1-ylmethyl]-7-methyl-4-morpholin-4-yl-thieno[3,2-d]pyrimidin-2-yl}-1H-indole-6-carbonitrile;
2-{4-[2-(6-fluoro-1H-indol-4-yl)-7-methyl-4-morpholin-4-yl-thieno[3,2-d]pyrimidin-6-ylmethyl]-piperazin-1-yl}-isobutyramide;
2-(6-methanesulfonyl-1H-indol-4-yl)-6-[4-(2-methoxy-ethyl)-piperidin-1-ylmethyl]-7-methyl-4-morpholin-4-yl-thieno[3,2-d]pyrimidine;
2-{4-[2-(6-cyano-1H-indol-4-yl)-7-methyl-4-morpholin-4-yl-thieno[3,2-d]pyrimidin-6-ylmethyl]-piperazin-1-yl}-isobutyramide;
2-{4-[2-(6-methanesulfonyl-1H-indol-4-yl)-7-methyl-4-morpholin-4-yl-thieno[3,2-d]pyrimidin-6-ylmethyl]-piperazin-1-yl}-isobutyramide;
2-{4-[2-(1H-indol-4-yl)-4-morpholin-4-yl-thieno[3,2-d]pyrimidin-6-ylmethyl]-piperazin-1-yl}-2-methyl-1-pyrrolidin-1-yl-propan-1-one;
cyclopropylmethyl-{1-[2-(1H-indol-4-yl)-4-morpholin-4-yl-thieno[3,2-d]pyrimidin-6-ylmethyl]-piperidin-4-yl}-(2-methoxy-ethyl)-amine;
2-(1H-indol-4-yl)-6-(4-isopropyl-piperazin-1-ylmethyl)-4-morpholin-4-yl-thieno[3,2-d]pyrimidine;
2-(1H-indol-4-yl)-6-(4-isopropyl-piperazin-1-ylmethyl)-4-morpholin-4-yl-thieno[2,3-d]pyrimidine;
6-[4-(2-methoxy-ethyl)-piperidin-1-ylmethyl]-4-morpholin-4-yl-2-(6-trifluoromethyl-1H-indol-4-yl)-thieno[3,2-d]pyrimidine;
2-(1H-indol-4-yl)-4-morpholin-4-yl-6-piperazin-1-ylmethyl-thieno[2,3-d]pyrimidine;
4-morpholin-4-yl-6-piperazin-1-ylmethyl-2-(6-trifluoromethyl-1H-indol-4-yl)-thieno[2,3-d]pyrimidine;
2-{4-[2-(1H-indol-4-yl)-4-morpholin-4-yl-thieno[2,3-d]primidin-6-ylmethyl]-piperazin-1-yl}-ethanol;
4-[6-(4-isopropyl-piperazin-1-ylmethyl)-4-morpholin-4-yl-thieno[2,3-d]pyrimidin-2-yl]-1H-indole-6-carbonitrile;
4-(4-morpholin-4-yl-6-piperazin-1-ylmethyl-thieno[2,3-d]pyrimidin-2-yl)-1H-indole-6-carbonitrile;
4-(4-morpholin-4-yl-6-piperidin-1-ylmethyl-thieno[3,2-d]pyrimidin-2-yl)-1H-indole-6-carboxylic acid amide;
4-(4-morpholin-4-yl-6-piperidin-1-ylmethyl-thieno[3,2-d]pyrimidin-2-yl)-1H-indole-6-sulfonic acid dimethylamide;
4-{6-[4-(2-methoxy-ethyl)-piperidin-1-ylmethyl]-4-morpholin-4-yl-thieno[3,2-d]pyrimidin-2-yl}-1H-indole-6-carboxylic acid amide;
4-(4-morpholin-4-yl-6-piperazin-1-ylmethyl-thieno[2,3-d]primidin-2-yl)-1H-indole-6-sulfonic acid dimethylamide;
4-(4-morpholin-4-yl-6-piperazin-1-ylmethyl-thieno[2,3-d]pyrimidin-2-yl)-1H-indole-6-carboxylic acid amide;
2-{4-[2-(6-methanesulfonyl-1H-indol-4-yl)-4-morpholin-4-yl-thieno[3,2-d]pyrimidin-6-ylmethyl]-piperazin-1-yl}-N-methyl-isobutyl amide;
2-(5-fluoro-1H-indol-4-yl)-4-morpholin-4-yl-6-piperidin-1-ylmethyl-thieno[3,2-d]pyrimidine;
4-(4-morpholin-4-yl-6-piperidin-1-ylmethyl-thieno[3,2-d]pyrimidin-2-yl)-1H-indole-2-carbonitrile;
4-{6-[4-(2-hydroxy-ethyp-piperazin-1-ylmethyl]-4-morpholin-4-yl-thieno[3,2-d]pyrimidin-2-yl}-1,1-indole-6-carbonitrile;
2-{4-[2-(6-methanesulfonyl-1H-indol-4-yl)-4-morpholin-4-yl-thieno[3,2-d]pyrimidin-6-ylmethyl]-piperazin-1-yl}-ethanol;
4-{6-[4-(2-Hydroxy-1,1-dimethyl-ethyl)-piperazin-1-ylmethyl]-4-morpholin-4-yl-thieno[3,2-d]pyrimidin-2-yl}-1H-indole-6-carbonitrile;

2-{4-[2-(6-fluoro-1H-indol-4-yl)-4-morpholin-4-yl-thieno[3,2-d]pyrimidin-6-ylmethyl]-piperazin-1-yl}-2-methyl-propan-1-ol;
4-morpholin-4-yl-6-piperidin-1-ylmethyl-2-(2-trifluoromethyl-1H-indol-4-yl)-thieno[3,2-d]pyrimidine;
6-[4-(2-methoxy-ethyl)-piperidin-1-ylmethyl]-4-morpholin-4-yl-2-(2-trifluoromethyl-1H-indol-4-yl)-thieno[3,2-d]pyrimidine;
4-morpholin-4-yl-6-piperazin-1-ylmethyl-2-(2-trifluoromethyl-1H-indol-4-yl)-thieno[2,3-d]pyrimidine;
4-morpholin-4-yl-6-piperidin-1-ylmethyl-2-(6-trifluoromethyl-1H-indol-4-yl)-thieno[3,2-d]pyrimidine;
2-(5-fluoro-1H-indol-4-yl)-4-morpholin-4-yl-6-piperazin-1-ylmethyl-thieno[2,3-d]pyrimidine;
4-(4-morpholin-4-yl-6-piperazin-1-ylmethyl-thieno[2,3-d]pyrimidin-2-yl)-1H-indole-2-carbonitrile;
4-(4-morpholin-4-yl-6-piperazin-1-ylmethyl-thieno[2,3-d]pyrimidin-2-yl)-1H-indole-2-carboxylic acid amide;
1-butoxy-3-{4-[2-(1H-indol-4-yl)-4-morpholin-4-yl-thieno[3,2-d]pyrimidin-6-ylmethyl]-piperazin-1-yl}-propan-2-ol;
6-(cis-3,5-dimethyl-piperazin-1-ylmethyl)-2-(6-fluoro-1H-indol-4-yl)-4-morpholin-4-yl-thieno[3,2-d]pyrimidine;
{1-[2-(6-fluoro-1H-indol-4-yl)-4-morpholin-4-yl-thieno[3,2-d]pyrimidin-6-ylmethyl]-pyrrolidin-3-yl}-dimethylamine;
2-(6-fluoro-1H-indol-4-yl)-6-(3-methyl-piperazin-1-ylmethyl)-4-morpholin-4-yl-thieno[3,2-d]pyrimidine;
1-[2-(6-fluoro-1H-indol-4-yl)-4-morpholin-4-yl-thieno[3,2-d]pyrimidin-6-ylmethyl]-piperidin-4-ylamine;
2-(6-fluoro-1H-indol-4-yl)-4-morpholin-4-yl-6-(4-pyrrolidin-1-yl-piperidin-1-ylmethyl)-thieno[3,2-d]pyrimidine;
{1-[2-(5-fluoro-1H-indol-4-yl)-4-morpholin-4-yl-thieno[3,2-d]pyrimidin-6-ylmethyl]-piperidin-4-yl}-dimethyl-amine;
{1-[2-(6-fluoro-1H-indol-4-yl)-4-morpholin-4-yl-thieno[3,2-d]pyrimidin-6-ylmethyl]-piperidin-4-yl}-dimethyl-amine;
2-{4-[2-(6-fluoro-1H-indol-4-yl)-4-morpholin-4-yl-thieno[3,2-d]pyrimidin-6-ylmethyl]-piperazin-1-N,N-dimethyl-isobutyramide;
{1-[2-(6-fluoro-1H-indol-4-yl)-4-morpholin-4-yl-thieno[3,2-d]pyrimidin-6-ylmethyl]-piperidin-3-yl}-dimethyl-amine;
2-(6-fluoro-1H-indol-4-yl)-6-((5)-3-isopropyl-piperazin-1-ylmethyl)-4-morpholin-4-yl-thieno[3,2-d]pyrimidine;
2-(1H-indol-4-yl)-6-[4-(2-methoxy-ethyl)-piperazin-1-ylmethyl]-4-morpholin-4-yl-thieno[3,2-d]pyrimidine;
3-{4-[2-(1H-indol-4-yl)-4-morpholin-4-yl-thieno[3,2-d]pyrimidin-6-ylmethyl]-piperazin-1-yl}-propan-1-ol;
3-{4-[2-(1H-indol-4-yl)-4-morpholin-4-yl-thieno[3,2-d]pyrimidin-6-ylmethyl]-piperazin-1-yl}-propionitrile;
2-{4-[2-(1H-indol-4-yl)-4-morpholin-4-yl-thieno[3,2-d]pyrimidin-6-ylmethyl]-piperazin-1-yl}-acetamide;
1-{4-[2-(1H-indol-4-yl)-4-morpholin-4-yl-thieno[3,2-d]pyrimidin-6-ylmethyl]-piperazin-1-yl}-propan-2-ol;
3-{4-[2-(1H-indol-4-yl)-4-morpholin-4-yl-thieno[3,2-d]pyrimidin-6-ylmethyl]-piperazin-1-yl}-propionamide;
6-(4-cyclobutylmethyl-piperazin-1-ylmethyl)-2-(1H-indol-4-yl)-4-morpholin-4-yl-thieno[3,2-d]pyrimidine;
N-cyclopropyl-2-{4-[2-(1H-indol-4-yl)-4-morpholin-4-yl-thieno[3,2-d]pyrimidin-6-ylmethyl]-piperazin-1-yl}-acetamide;
6-[4-(2,6-dichloro-pyridin-4-ylmethyl)-piperazin-1-ylmethyl]-2-(1H-indol-4-yl)-4-morpholin-4-yl-thieno[3,2-d]pyrimidine;
2-(1H-indol-4-yl)-4-morpholin-4-yl-6-(4-propyl-piperazin-1-ylmethyl)-thieno[3,2-d]pyrimidine;
1-{4-[2-(1H-indol-4-yl)-4-morpholin-4-yl-thieno[3,2-d]pyrimidin-6-ylmethyl]-piperazin-1-yl}-3,3-dimethyl-butan-2-one;
2-(1H-indol-4-yl)-6-(4-isobutyl-piperazin-1-ylmethyl)-4-morpholin-4-yl-thieno[3,2-d]pyrimidine;
2-{4-[2-(1H-indol-4-yl)-4-morpholin-4-yl-thieno[3,2-d]pyrimidin-6-ylmethyl]-piperazin-1-yl}-ethylamine;
Diethyl-(2-{4-[2-(1H-indol-4-yl)-4-morpholin-4-yl-thieno[3,2-d]pyrimidin-6-ylmethyl]-piperazin-1-yl}-ethyl)-amine;
6-(4-ethyl-piperazin-1-ylmethyl)-2-(1H-indol-4-yl)-4-morpholin-4-yl-thieno[3,2-d]pyrimidine;
2-(1H-indol-4-yl)-6-(4-methyl-piperidin-1-ylmethyl)-4-morpholin-4-yl-thieno[3,2-d]pyrimidine;
2-(1H-indol-4-yl)-6-(3-methyl-piperidin-1-ylmethyl)-4-morpholin-4-yl-thieno[3,2-d]pyrimidine;
6-(3,5-dimethyl-piperidin-1-ylmethyl)-2-(1H-imdol-4-yl)-4-morpholin-4-yl-thieno[3,2-d]pyrimidine;
6-(2-ethyl-piperidin-1-ylmethyl)-2-(1H-indol-4-yl)-4-morpholin-4-yl-thieno[3,2-d]pyrimidine;
{1-[2-(1H-indol-4-yl)-4-morpholin-4-yl-thieno[3,2-d]pyrimidin-6-ylmethyl]-piperidin-3-yl}-methanol;
2-(1H-indol-4-yl)-6-(2-methyl-piperidin-1-ylmethyl)-4-morpholin-4-yl-thieno[3,2-d]pyrimidine;
2-(1H-indol-4-yl)-4-morpholin-4-yl-6-[4-(3-piperidin-1-yl-propyl)-piperazin-1-ylmethyl]-thieno[3,2-d]pyrimidine;
2-(1H-indol-4-yl)-4-morpholin-4-yl-6-(4-pyridin-2-ylmethyl-piperazin-1-ylmethyl)-thieno[3,2-d]pyrimidine;
4-{4-[2-(1H-indol-4-yl)-4-morpholin-4-yl-thieno[3,2-d]pyrimidin-6-ylmethyl]-piperazin-1-yl}-butyronitrile;
2-(1H-indol-4-yl)-4-morpholin-4-yl-6-piperidin-1-ylmethyl-thieno[3,2-d]pyrimidine;
2-(1H-indol-4-yl)-6-(2-methyl-pyrrolidin-1-ylmethyl)-4-morpholin-4-yl-thieno[3,2-d]pyrimidine;
2-(1H-indol-4-yl)-4-morpholin-4-yl-6-(4-pyridin-2-yl-piperazin-1-ylmethyl)-thieno[3,2-d]pyrimidine;
{1-[1H-indol-4-yl)-4-morpholin-4-yl-thieno[3,2-d]pyrimidin-6-ylmethyl]-pyrrolidin-3-yl}-methanol;
2-(1H-indol-4-yl)-6-{4-[2-(1-methyl-pyrrolidin-2-yl)-ethyl]-piperazin-1-ylmethyl}-4-morpholin-4-yl-thieno[3,2-d]pyrimidine;
2-(1H-indol-4-yl)-4-morpholin-4-yl-6-[4-(2-piperidin-1-yl-ethyl)-piperazin-1-ylmethyl]-thieno[3,2-d]pyrimidine;
2-(1H-indol-4-yl)-4-morpholin-4-yl-6-[4-(2-pyrrolidin-1-yl-ethyl)-piperazin-1-ylmethyl]-thieno[3,2-d]pyrimidine;
6-(4-cyclopropylmethyl-piperazin-1-ylmethyl)-2-(1H-indol-4-yl)-4-morpholin-4-yl-thieno[3,2-d]pyrimidine;
6-(cis-3,5-dimethyl-piperazin-1-ylmethyl)-2-(1H-indol-4-yl)-4-morpholin-4-yl-thieno[3,2-d]pyrimidine;
6-(cis-3,5-dimethyl-piperazin-1-ylmethyl)-2-(5-fluoro-1H-indol-4-yl)-4-morpholin-4-yl-thieno[3,2-d]pyrimidine;
{1-[2-(6-fluoro-1H-indol-4-yl)-4-morpholin-4-yl-thieno[3,2-d]primidin-6-ylmethyl]-piperidin-4-yl}-methyl-amine; and
2-(6-fluoro-1H-indol-4-yl)-6-((R)-3-isopropyl-piperazin-1-ylmethyl)-4-morpholin-4-yl-thieno[3,2-d]pyrimidine;

as well as the pharmaceutically acceptable salts and/or hydrates of any of the foregoing compounds.

The drug IC87114, which is in phase one clinical trials for the treatment of hematological cancers (ClinicalTrials.gov Identifier: NCT00710528), has not been tried in the treatment of CNS disorders, nor is its patent based on an indication for CNS uses. We have discovered evidence that this drug and other PIK3CD inhibitors will be effective treatments of psychosis and cognitive decline. Because psychosis and cognitive decline are among the most common and debilitating afflictions of humans, the search for new treatments is very important and timely. PIK3CD is a druggable target, and a drug already exists that affects this enzyme and is being tested in the context of other medical disorders.

In one embodiment, a selective PIK3CD inhibitor is an antibody. The present disclosure includes isolated (i.e., removed from their natural milieu) antibodies that selectively bind PIK3CD. As used herein, the term “selectively binds to” refers to the ability of antibodies of the present disclosure to preferentially bind to PIK3CD. Binding can be measured using a variety of methods standard in the art including enzyme immunoassays (e.g., ELISA), immunoblot assays, and the like; see, for example, Sambrook et al., Eds., Molecular Cloning: A Laboratory Manual, 2nd ed., Cold Spring Harbor Laboratory Press, 1989, or Harlow and Lane, Eds., Using Antibodies, Cold Spring Harbor Laboratory Press, 1999. An antibody selectively binds to or complexes with PIK3CD, preferably in such a way as to reduce the activity of PIK3CD.

As used herein, antibody includes antibodies in serum, or antibodies that have been purified to varying degrees, specifically at least about 25% homogeneity. The antibodies are specifically purified to at least about 50% homogeneity, more specifically at least about 75% homogeneity, and most specifically greater than about 90% homogeneity. Antibodies may be polyclonal antibodies, monoclonal antibodies, humanized or chimeric antibodies, anti-idiotypic antibodies, single chain antibodies, Fab fragments, fragments produced from an Fab expression library, epitope-binding fragments of the above, and the like. An antibody includes a biologically active fragment, that is, a fragment of a full-length antibody the same target as the full-length antibody. Biologically active fragments include Fab, F(ab′)₂ and Fab′ fragments.

Antibodies are prepared by immunizing an animal with full-length polypeptide or fragments thereof. The preparation of polyclonal antibodies is well known in the molecular biology art; see for example, Production of Polyclonal Antisera in Immunochemical Processes (Manson, ed.), (Humana Press 1992) and Coligan et al., Production of Polyclonal Antisera in Rabbits, Rats, Mice and Hamsters in Current Protocols in Immunology, (1992).

A monoclonal antibody composition is produced, for example, by clones of a single cell called a hybridoma that secretes or otherwise produces one kind of antibody molecule. Hybridoma cells are formed, for example, by fusing an antibody-producing cell and a myeloma cell or other self-perpetuating cell line. Numerous variations have been described for producing hybridoma cells.

In one embodiment, monoclonal antibodies are obtained by injecting mammals such as mice or rabbits with a composition comprising an antigen, thereby inducing in the animal antibodies having specificity for the antigen. A suspension of antibody-producing cells is then prepared (e.g., by removing the spleen and separating individual spleen cells by methods known in the art). The antibody-producing cells are treated with a transforming agent capable of producing a transformed or “immortalized” cell line. Transforming agents are known in the art and include such agents as DNA viruses (e.g., Epstein Bar Virus, SV40), RNA viruses (e.g., Moloney Murine Leukemia Virus, Rous Sarcoma Virus), myeloma cells (e.g., P3X63-Ag8.653, Sp2/0-Ag14), and the like. Treatment with the transforming agent results in production of a hybridoma by means of fusing the suspended spleen cells with, for example, mouse myeloma cells. The transformed cells are then cloned, preferably to monoclonality. The cloning is performed in a medium that will not support non-transformed cells, but that will support transformed cells. The tissue culture medium of the cloned hybridoma is then assayed to detect the presence of secreted antibody molecules by antibody screening methods known in the art. The desired clonal cell lines are then selected.

A therapeutically useful antibody may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions.

In addition, chimeric antibodies can be obtained by splicing the genes from a mouse antibody molecule with appropriate antigen specificity together with genes from a human antibody molecule of appropriate biological specificity. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies are used to produce single chain antibodies, as described, for example, in U.S. Pat. No. 4,946,778. Single chain antibodies are formed by linking the heavy and light chain fragments of the Fv region via an amino acid bridge, resulting in a single chain polypeptide.

In one embodiment, antibody fragments that recognize specific epitopes are generated by techniques well known in the art. Such fragments include Fab and F(ab′)₂ fragments produced by proteolytic digestion, and Fab′ fragments generated by reducing disulfide bridges. Fab, F(ab′)₂ and Fab′ fragments of antibodies can be prepared. Fab fragments are typically about 50 kDa, while F(ab′)₂ fragments are typically about 100 kDa in size. Antibodies are isolated (e.g., on protein G columns) and then digested and purified with sepharose coupled to papain and to pepsin in order to purify Fab and F(ab′)₂ fragments according to protocols provided by the manufacturer (Pierce Chemical Co.). The antibody fragments are further purified, isolated and tested using ELISA assays. Antibody fragments are assessed for the presence of light chain and Fc epitopes by ELISA.

In another embodiment, antibodies are produced recombinantly using techniques known in the art. Recombinant DNA methods for producing antibodies include isolating, manipulating, and expressing the nucleic acid that codes for all or part of an immunoglobulin variable region including both the portion of the variable region comprised by the variable region of the immunoglobulin light chain and the portion of the variable region comprised by the variable region of the immunoglobulin heavy chain. Methods for isolating, manipulating and expressing the variable region coding nucleic acid in eukaryotic and prokaryotic subjects are known in the art.

The structure of the antibody may also be altered by changing the biochemical characteristics of the constant regions of the antibody molecule to a form that is appropriate to the particular context of the antibody use. For example, the isotype of the antibody may be changed to an IgA form to make it compatible with oral administration. IgM, IgG, IgD, or IgE isoforms may have alternate values in the specific therapy in which the antibody is used.

Antibodies are purified by methods known in the art. Suitable methods for antibody purification include purification on Protein A or Protein G beads, protein chromatography methods (e.g., DEAE ion exchange chromatography, ammonium sulfate precipitation), antigen affinity chromatography and others.

In one embodiment, the selective PIK3CD inhibitor comprises an antisense RNA. An antisense RNA (aRNA) is single-stranded RNA that is complementary to a messenger RNA (mRNA) strand transcribed within a cell. Antisense RNA may be introduced into a cell to inhibit translation of a complementary mRNA by base pairing to it and physically obstructing the translation machinery. An antisense molecule specific for an S1P₂ receptor should generally be substantially identical to at least a portion, specifically at least about 20 continuous nucleotides, of the nucleic acid encoding the S1P₂ receptor, but need not be identical. The antisense nucleic acid molecule can be designed such that the inhibitory effect applies to other proteins within a family of genes exhibiting homology or substantial homology to the nucleic acid. The introduced antisense nucleic acid molecule also need not be full-length relative to either the primary transcription product or fully processed mRNA. Generally, higher homology can be used to compensate for the use of a shorter sequence. Furthermore, the antisense molecule need not have the same intron or exon pattern, and homology of non-coding segments will be equally effective. Antisense phosphorothioate oligodeoxynucleotides (PS-ODNs) is exemplary of an antisense molecule specific for the S1P₂ receptor.

In another embodiment, the selective PIK3CD inhibitor comprises an siRNA. RNA interference (“RNAi”) is a method of post-transcriptional gene regulation that is conserved throughout many eukaryotic organisms. RNAi is induced by short (i.e., less than 30 nucleotide) double stranded RNA (“dsRNA”) molecules, which are present in the cell. These short dsRNA molecules, called “short interfering RNA” or “siRNA”, cause the destruction of messenger RNAs (“mRNAs”), which share sequence homology with the siRNA to within one nucleotide resolution. Without being held to theory, it is believed that the siRNA and the targeted mRNA bind to an “RNA-induced silencing complex” or “RISC”, which cleaves the targeted mRNA. The siRNA is apparently recycled much like a multiple-turnover enzyme, with 1 siRNA molecule capable of inducing cleavage of approximately 1000 mRNA molecules. siRNA-mediated RNAi degradation of an mRNA is therefore effective for inhibiting expression of a target gene.

siRNA comprises short double-stranded RNA of about 17 nucleotides to about 29 nucleotides in length, specifically about 19 to about 25 nucleotides in length, that are targeted to the target mRNA, that is, PIK3CD mRNA. The siRNA comprise a sense RNA strand and a complementary antisense RNA strand annealed together by standard Watson-Crick base-pairing interactions (“base-paired”). The sense strand comprises a nucleic acid sequence which is identical to a target sequence contained within the target mRNA.

The sense and antisense strands of siRNA comprise two complementary, single-stranded RNA molecules, or comprise a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form an siRNA of two individual base-paired RNA molecules.

One or both strands of the siRNA can also comprise a 3′ overhang. A “3′ overhang” refers to at least one unpaired nucleotide extending from the 3′-end of a duplexed RNA strand. In one embodiment, the siRNA comprises at least one 3′ overhang of 1 to about 6 nucleotides (which includes ribonucleotides or deoxynucleotides) in length, specifically of 1 to about 5 nucleotides in length, more specifically of 1 to about 4 nucleotides in length, and particularly specifically of about 2 to about 4 nucleotides in length. In the embodiment in which both strands of the siRNA molecule comprise a 3′ overhang, the length of the overhangs can be the same or different for each strand. In one embodiment, the 3′ overhang is present on both strands of the siRNA, and is 2 nucleotides in length. For example, each strand of the siRNA of the can comprise 3′ overhangs of dithymidylic acid (“TT”) or diuridylic acid (“uu”). In order to enhance the stability of the siRNA, the 3′ overhangs can also be stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′; -deoxythymidine significantly enhances the nuclease resistance of the 3′ overhang in tissue culture medium.

The siRNA is obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference. The siRNA expressed from recombinant plasmids is isolated from cultured cell expression systems by standard techniques, or is expressed intracellularly at or near the area of neovascularization in vivo. The siRNA can also be expressed from recombinant viral vectors intracellularly. The recombinant viral vectors comprise sequences encoding the siRNA and a promoter for expressing the siRNA sequences. Exemplary promoters include, for example, the U6 or H1 RNA pol III promoter sequences and the cytomegalovirus promoter.

One skilled in the art can readily determine an effective amount of the siRNA to be administered to a given subject, by taking into account factors such as the size and weight of the subject; the extent of the disorder; the age, health and sex of the subject; the route of administration; and whether the administration is regional or systemic.

In one embodiment, the therapeutic value of a selective PIK3CD inhibitor can be predicted by ErbB4 or PIK3CD genotype, the disease state and or the cognitive function. Peripheral and/or CNS ErbB4 and PIK3CD levels and NRG1 induced PIP3 production serve as biomarkers that may be useful in predicting response. Thus, in certain embodiments, the method of administering selective PIK3CD inhibitors to treat CNS disorders further comprise additional steps such as determining an ErB4 genotype of the individual, determining the PIK3CD genotype of the individual, determining the disease state of the individual, determining the cognitive function of the individual, determining the peripheral and/or CNS Erb4 level in the individual, determining the peripheral and/or CNS PIK3CD level in the individual, and/or determining NRG1-induced PIP3 production.

In an embodiment, determining the ErbB4 genotype of an individual comprises determining the diplotype of the individual for a risk-associated haplotype comprising three single nucleotide polymorphisms (SNPs) in ErbB4, rs7598440; rs839523; and rs707284 and the risk-associated haplotype has the alleles AGG at rs7598440; rs839523; and rs707284, respectively. All SNPs herein are referred to by reference SNP identifier from National Center for Biotechnology Information dbSNP, build 130.

In another embodiment, determining the ErbB4 genotype of an individual comprises determining the allele present on one or both chromosomes in the individual of any of the ErbB4 SNPs enumerated in Table 2.

In one embodiment, determining the PIK3CD genotype of an individual comprises identifying the allele present on one or both chromosomes in the individual of any of the 20 PIC3CD SNPs shown in Table 1.

The SNPs in Tables 1 and 2 are identified by the reference SNP identifier (rs number) of the SNP in the NCBI dbSNP database. For each SNP of a unique rs number in the database, a reference sequence and a position of the SNP within that reference sequence is provided. Those skilled in the art may easily identify the reference sequence and the position of the SNP using the dbSNP rs Accession No. All or only part of the reference sequence flanking the polymorphic site can be used by the skilled practitioner to identify the SNP in a nucleic acid. The column labeled SEQ ID NO. in Tables 1 and 2 presents a sequence identification number for the reference sequence provided by dbSNP build 130 for identification of the listed SNP in a nucleic acid. The position of the polymorphic site in the SEQ ID NO. is also provided. In describing the SNPs herein, reference is made to the reference sequence for convenience. However, as recognized by the skilled artisan, nucleic acid molecules containing a particular gene such as PIK3CD or ErbB4 may be complementary double stranded molecules and thus reference to alleles at a particular SNP or haplotype on the reference sequence refers as well to the complementary alleles at the SNP or haplotype on the complementary strand. Further, reference may be made to detecting a genetic marker or haplotype for one strand and it will be understood by the skilled artisan that this includes detection of the complementary allele on the other strand.

The term “genotype” refers to a description of the alleles of a gene or genes contained in an individual. As used herein, no distinction is made between the genotype of an individual and the genotype of a sample originating from the individual. Although, typically, a genotype is determined from samples of diploid cells, a genotype can be determined from a sample of haploid cells, such as a sperm cell. The term “haplotype” refers to a combination of alleles, for example at one or more polymorphic sites, that are located together on the same chromosome and that tend to be inherited together. A “diplotype” is the pair of haplotypes for one or more polymorphic sites characterizing both chromosomes of an individual. The term “target region” refers to a region of a nucleic acid that is to be analyzed and usually includes at least one polymorphic region. “Linkage Disequilibrium” (“LD”) refers to alleles at different loci that are not associated at random, i.e., not associated in proportion to their frequencies. If the alleles are in positive linkage disequilibrium, then the alleles occur together more often than expected assuming statistical independence. Conversely, if the alleles are in negative linkage disequilibrium, then the alleles occur together less often than expected assuming statistical independence.

The individual's genotype for a polymorphic site may be determined using a variety of methods well known in the art for identifying the nucleotide present at polymorphic sites. The particular method used to identify the genotype is not a critical aspect of the invention. Although considerations of performance, cost, and convenience will make particular methods more desirable than others, it will be clear that any method that can identify the nucleotide present will provide the information needed to identify the genotype. Examples of genotyping methods include DNA sequencing, allele-specific amplification, or probe-based detection of amplified nucleic acid.

Such methods often include isolating a genomic DNA sample from the individual comprising both copies of the gene or locus of interest, amplifying from the sample one or more target regions containing the polymorphic sites to be genotyped, and detecting the nucleotides present at each polymorphic site of interest in the amplified target region(s).

Alleles can be identified by DNA sequencing methods, such as the chain termination method (Sanger et al., 1977, Proc. Natl. Acad. Sci., 74:5463 5467), which are well known in the art. In one embodiment, a subsequence of the gene encompassing the polymorphic site is amplified and either cloned into a suitable plasmid and then sequenced, or sequenced directly. PCR-based sequencing is described in U.S. Pat. No. 5,075,216. Typically, sequencing is carried out using one of the automated DNA sequencers that are commercially available, e.g., from Applied Biosystems (Foster City, Calif.)

Genotyping alleles can also be performed using amplification-based genotyping methods. Various nucleic acid amplification methods known in the art can be used in to detect nucleotide changes in a target nucleic acid. A preferred method is the polymerase chain reaction (PCR), which is now well known in the art, and described in U.S. Pat. Nos. 4,683,195; 4,683,202; and 4,965,188. Commercial vendors, such as Applied Biosystems (Foster City, Calif.) market PCR reagents and publish PCR protocols.

Other suitable amplification methods include the ligase chain reaction; the strand displacement assay; and several transcription-based amplification systems, including the methods described in U.S. Pat. Nos. 5,437,990; 5,409,818; and 5,399,491; the transcription amplification system (TAS); and self-sustained sequence replication (3SR) (WO 92/08800). Alternatively, methods that amplify the probe to detectable levels can be used, such as Qβ-replicase amplification.

Genotyping also can also be carried out by detecting and analyzing mRNA under conditions when both, maternal and paternal, chromosomes are transcribed. Amplification of RNA can be carried out by first reverse-transcribing the target RNA using, for example, a viral reverse transcriptase, and then amplifying the resulting cDNA, or using a combined high-temperature reverse-transcription-polymerase chain reaction (RT-PCR), as described in U.S. Pat. Nos. 5,310,652; 5,322,770; 5,561,058; 5,641,864; and 5,693,517.

Alleles can also be identified using allele-specific amplification or primer extension methods, which are based on the inhibitory effect of a terminal primer mismatch on the ability of a DNA polymerase to extend the primer. To detect an allele sequence using an allele-specific amplification or extension-based method, a primer complementary to the target region is chosen such that the 3′ terminal nucleotide hybridizes at the polymorphic position. In the presence of the allele to be identified, the primer matches the target sequence at the 3′ terminus and primer is extended. In the presence of only the other allele, the primer has a 3′ mismatch relative to the target sequence and primer extension is either eliminated or significantly reduced. Allele-specific amplification- or extension-based methods are described in, for example, U.S. Pat. Nos. 5,137,806; 5,595,890; 5,639,611; and U.S. Pat. No. 4,851,331.

Using allele-specific amplification-based genotyping, identification of the alleles requires only detection of the presence or absence of amplified target sequences. Methods for the detection of amplified target sequences are well known in the art. For example, gel electrophoresis and the probe hybridization assays described above have been used widely to detect the presence of nucleic acids.

Alleles can be also identified using probe-based methods, which rely on the difference in stability of hybridization duplexes formed between a probe and its corresponding target sequence comprising a polymorphic site. Under sufficiently stringent hybridization conditions, stable duplexes are formed only between a probe and its target allele sequence and not other allele sequences. The presence of stable hybridization duplexes can be detected by any of a number of well known methods. In general, it is preferable to amplify a nucleic acid encompassing a polymorphic site of interest prior to hybridization in order to facilitate detection. However, this is not necessary if sufficient nucleic acid can be obtained without amplification.

Probe-based genotyping can be carried out using a “TaqMan®” or “5′-nuclease assay”, as described in U.S. Pat. Nos. 5,210,015; 5,487,972; and 5,804,375. In the TaqMan® assay, labeled detection probes that hybridize within the amplified region are added during the amplification reaction mixture. The probes are modified so as to prevent the probes from acting as primers for DNA synthesis. The amplification is carried out using a DNA polymerase that possesses 5′ to 3′ exonuclease activity, e.g., Tth DNA polymerase. During each synthesis step of the amplification, any probe which hybridizes to the target nucleic acid downstream from the primer being extended is degraded by the 5′ to 3′ exonuclease activity of the DNA polymerase. Thus, the synthesis of a new target strand also results in the degradation of a probe, and the accumulation of degradation product provides a measure of the synthesis of target sequences. Any method suitable for detecting degradation product can be used in the TaqMan® assay. In some embodiments, the accumulation of degradation product is monitored by measuring the increase in reaction fluorescence.

In addition, the identity of the allele(s) present at a polymorphic site described herein may be indirectly determined by haplotyping or genotyping another polymorphic site having an allele that is in linkage disequilibrium with an allele of the polymorphic site that is of interest. Detection of the allele(s) present at a polymorphic site, wherein the allele is in linkage disequilibrium with an allele of the novel polymorphic sites described herein may be performed by, but is not limited to, any of the above-mentioned methods for detecting the identity of the allele at a polymorphic site.

The nucleic acid sample used in the above genotyping methods is typically isolated from a biological sample taken from the individual, such as a blood sample or tissue sample. Suitable tissue samples include whole blood, saliva, tears, urine, skin, and hair.

In both direct and indirect haplotyping methods, the identity of a nucleotide at a polymorphic site(s) in the amplified target region may be determined by sequencing the amplified region(s) using conventional methods. If both copies of the gene are represented in the amplified target, it will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a polymorphism is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

A polymorphic site in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated, or derivatized to facilitate the immobilization of the allele-specific oligonucleotide or target nucleic acid.

Detecting the nucleotide or nucleotide pair at a polymorphic site of interest may also be determined using a mismatch detection technique, including but not limited to the RNase protection method using riboprobes and proteins which recognize nucleotide mismatches, such as the E. coli muts protein. Alternatively, variant alleles can be identified by single strand conformation polymorphism (SSCP) analysis.

A polymerase-mediated primer extension method may also be used to identify the polymorphism(s). Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798. Another primer extension method is allele-specific PCR. In addition, multiple polymorphic sites may be investigated by simultaneously amplifying multiple regions of the nucleic acid using sets of allele-specific primers as described in WO 89/10414.

The genotype or haplotype of an individual may also be determined by hybridization of a nucleic acid sample containing one or both copies of the gene, mRNA, cDNA or fragment(s) thereof, to nucleic acid arrays and subarrays such as described in WO 95/11995. The arrays would contain a battery of allele-specific oligonucleotides representing each of the polymorphic sites to be included in the genotype or haplotype.

Phasing of genotype information into haplotypes can be performed statistically using commercially available or free software packages.

Direct haplotyping of an individual can be performed using a method such as, for example, CLASPER System™ technology ((U.S. Pat. No. 5,866,404), single molecule dilution, or allele-specific long-range PCR (Michalotos-Beloin et al., Nucleic Acids Res. 24:4841-3 (1996)).

In one embodiment, the method comprises, prior to administering the selective PIK3CD inhibitor, determining the disease state and/or the cognitive function of the individual.

In one embodiment, the method further comprises determining for the individual a cognitive factor score, where in the cognitive factor score includes verbal memory, digit span, processing speed, visual memory, attention, card sorting, or a combination thereof. One useful cognitive test is the Measurement and Treatment Research to Improve Cognition in Schizophrenia (MATRICS) Consensus Cognitive Battery (MCCB). The Schizophrenia Cognition Rating Scale is an 18-item interview-based assessment that covers all the cognitive domains tested in the MCCB, except social cognition. The 7 cognitive domains were speed of processing, attention/vigilance, working memory, verbal learning, visual learning, reasoning, and problem solving, and social cognition. The 5 selection criteria were reliability, utility, relationship to functional status, potential changeability in response to pharmacological agents, and practicality for clinical trials and tolerability for patients. Other cognitive tests for schizophrenia include the Wechsler Adult Intelligence Scale, the University of California San Diego Performance-Based Skills Assessment (UPSA), the Repeatable Battery for the Assessment of Neuropsychological Status (RBANS), the Brief Assessment of Cognition in Schizophrenia (BACS), and the Brief Cognitive Assessment (BCA).

In one embodiment, a neurocognitive battery comprised of neuropsychological tests with evidence of heritability and association with risk for schizophrenia is performed. It included the Wechsler Memory Scale-Revised (WMS-R), Wechsler Adult Intelligence Scale-Revised (WAIS-R: arithmetic, similarities, digit-symbol-substitution and picture completion), Trailmaking Test Parts A and B, Verbal and Category Fluency, Continuous Performance Test (CPT), N-Back task, California Verbal Learning Test (CVLT), Judgment of Line Orientation, and the Wisconsin Card Sorting Test (WCST). These 24 sub-tests are reducible via principal components and confirmatory factor analyses to a 7-factor solution. Factor 1 is loaded with verbal episodic memory measures from the WMS-R and CVLT; factor 2 with aspects of working memory from the N-back task; factor 3 with spatial episodic memory measures from WMS-R and Judgment of Line Orientation; factor 4 with executive cognitive control and processing speed measures from WAIS-R, trails A and B, and letter and category fluency; factor 5 with logical reasoning measures from the WCST, factor 6 with attention measures from the CPT, and factor 7 with measures from the WMS-R digit span backwards and forwards.

The PIK3CD inhibitors can be administered as the neat chemical, but are specifically administered as a pharmaceutical composition, for example a pharmaceutical formulation comprising a PIK3CD inhibitor or pharmaceutically acceptable salt and/or solvate (e.g., hydrate) thereof, together with at least one pharmaceutically acceptable carrier.

The PIK3CD inhibitor may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorings, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active and/or inactive agents may be included in the pharmaceutical compositions, provided that such agents do not substantially interfere with the activity of the PIK3CD inhibitors used in the pharmaceutical compositions. The optional active is an additional active agent that is not a compound or salt of formula I.

The pharmaceutical compositions can be formulated for oral administration. These compositions contain between 0.1 and 99 weight % (wt. %) of a 2 PIK3CD inhibitor and usually at least about 5 wt. % of a PIK3CD inhibitor. Some embodiments contain from about 25 wt. % to about 50 wt. % or from about 5 wt. % to about 75 wt. % of the PIK3CD inhibitor.

In one embodiment, the PIK3CD inhibitor is administered with a second active agent such as an antipsychotic or a mood stabilizer. Exemplary antipsychotic drugs include, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof. Exemplary mood stabilizers include carbamazepine, divalproex, gabapentin, lamotrigine, lithium, olanzapine, quetiapine, valproate, valproic acid, verapamil, and equivalents and pharmaceutically active isomer(s) and metabolite(s) thereof.

The methods disclosed herein are suitable for alleviating one or more symptoms of a variety of CNS disorders. Individuals with a CNS disorder frequently exhibit one or more symptoms that are characteristic of the particular disorder. It is also contemplated that a constellation of symptoms from multiple CNS disorders in the same individual can be alleviated by the present methods. In this regard, recognizing symptoms from CNS disorders, and determining alleviation of the symptoms during or after practice of the present method is well within the purview of a person having ordinary skill in the art and can be performed using any suitable clinical, diagnostic, observational, or other techniques. For example, symptoms of schizophrenia include but are not limited to delusions, hallucinations, disorganized speech, catatonic behavior, cognitive symptoms, or a combination thereof. Symptoms of psychosis include delusions, hallucinations, or a combination thereof. A reduction in any of these particular symptoms resulting from practicing the methods disclosed herein is considered an alleviation of the symptom. Particular CNS disorders presenting symptoms suitable for alleviation by the present methods include but are not limited to: broad spectrum psychosis such as bipolar disorders; depression; mood disorders; anxiety; obsessive compulsive disorders; sleep disorders; feeding disorders such as anorexia and bulimia; panic attacks; drug addictions and withdrawal from drug addictions; attention deficit disorders; cognitive disorders; age-associated memory impairment (AAMI); neurodegenerative disorders such as Alzheimer's disease, Parkinson's disease, and stroke related dementia; Down's Syndrome; and combinations thereof. Symptoms of each of these disorders are well known. Recognizing and determining a reduction in the symptoms of any of these particular disorders can be readily performed by those skilled in the art. In specific embodiments, the CNS disorder is schizophrenia, psychosis or a cognitive disorder.

### EXAMPLES

### Example 1

### Schizophrenia and Disease-Associated Polymorphisms in ErbB4 Predict Elevated Expression of a PI3K Signaling Complex

Lymphoblastoid B cell lines (LCLs) derived from patients with schizophrenia and normal control individuals were used to study the genetic regulation of the NRG1-ErbB4 signaling pathway in this example.

The human LCL system is a model for examination of the genetic regulation of NRG1-ErbB4 signaling in schizophrenia that replicates, in separate individuals, the impact of ErbB4 risk polymorphisms on ErbB4 CYT-1 expression seen in brain. Human LCLs predominantly express the ErbB4 isoform JM-a/CYT-1 ErbB4 receptor while expression of the JM-biCYT-2 isoform is undetectable. Thus, the ErbB4 isoform, specifically elevated in the brain in schizophrenia and regulated by a schizophrenia-associated haplotype in ErbB4 (AGG; rs7598440; rs839523; rs707284), can be studied in this cell type. LCLs also expressed abundant ErbB2 and ErbB3, the two other receptors that mediate NRG1 signaling. ErbB2 and ErbB3 expression was not associated with the ErbB4 risk genotype, but ErbB3 was significantly reduced in LCLs in schizophrenia as reported in human brain.

The human LCL cohort used was derived from 34 normal controls (18 females, 16 males; age 32.92 years at the time of blood collection and 25 individuals with schizophrenia (11 females, 14 males; age 37.6 years). All subjects were drawn from individuals participating in the Clinical Brain Disorders Branch “Sibling Study” (CDBD SS) protocol, an ongoing investigation of neurobiological abnormalities related to genetic risk for schizophrenia. Only Caucasian subjects of self-reported European ancestry were included to avoid genetic stratification and to reduce heterogeneity.

Expression of PIK3CA, PIK3CB, PIK3CD, PIK3R1, PIK3R2, and PIK3R3 gene transcripts was quantified in human LCLs.

For the expression studies, RNA from 32 normal controls and 23 patients was available. Total RNA was extracted from B lymphoblasts. Yield was determined by absorbance at 260 nm. RNA quality was assessed by high resolution capillary electrophoresis on an Agilent Bioanalyzer 2100 (Agilent Technologies Palo Alto, Calif., USA). Approximately 700 ng RNA was applied to a RNA 6000 Nano Lab Chip without prior heating. RNA integrity number (RIN), obtained from the entire Agilent electrophoretic trace using the RIN software algorithm, was used for assessment of RNA quality (scale 1-10, with 1 being the lowest and 10 being the highest RNA quality).

Total RNA (3 pg) was used in 50 μL of reverse transcriptase reaction to synthesize cDNA, by using a Superscript First-Strand Synthesis System for RT-PCR (Invitrogen, Carlsbad, Calif., USA) according the manufacturer's protocol. To control for potential variability between reverse transcriptase reactions, a total of 3 sequential reactions were performed (3 μg total RNA each) and the products pooled.

Gene expression levels were measured by quantitative real-time RT-PCR using an ABI Prism 7900 sequence detection system with 384-well format (Applied Biosystems, Foster City, Calif. USA). Briefly, each 20 μL reaction contained 900 nM of each primer, 250 nM of probe and TaqMan® Universal PCR Mastermix (Applied Biosystems) containing Hot Goldstar® DNA Polymerase, dNTPs with dUTP, uracil-N-glycosylase, passive reference and 200 ng of cDNA template. PCR cycle parameters were 50° C. for 2 min, 95° C. for 10 min, 40 cycles of 95° C. for 15s, and 59° C. or 60° C. for 1 min. PCR data were acquired from the Sequence Detector Software (SDS version 2.0, Applied Biosystems) and quantified by a standard curve method. In each experiment the R² value of the curve was more than 0.99 and controls comprising no-template cDNA resulted in no detectable signal. SDS software plotted real-time fluorescence intensity and selected the threshold within the exponential phase of the amplicon profiles. The software plotted a standard curve of the cycles at threshold (Ct) vs. quantity of RNA. For each target isoform, all samples were measured with constant reaction conditions and their Ct values were in the linear range of the standard curve. All measurements were performed in triplicates for each mRNA and expression level calculated as an average of the triplicates. Experimental measurements with a >20% variance from the mean of the triplicate samples were omitted.

Briefly, TaqMan® probes were designed to differentiate ErbB4 isoforms through hybridizing to isoform-specific exons, 16 or 15 JM-a, JM-b respectively and exon 26 for CYT-I. TaqMan® assay-on-demand sets were purchased from Applied Biosystems: hCG2012284 used for total ErbB4; Hs00908671 and Mm00435674 for PIK3CD; Hs00177524, PIK3R3; Hs01001599, ErbB2 and Hs00176538 ErbB3. Primary data analysis is based on normalization of mRNA transcripts to the geometric mean of the quantity of three endogenous control genes purchased from Applied Biosystems, Assays-on-demand: porphobilinogen deaminase (PBGD), glyceraldehydes-3-phosphate dehydrogenase (GADPH) and B-Actin (ACTBH) assays Hs00609297; Hs99999905 and Hs99999903, respectively.

In the genotyping experiments, all genotypes were determined using the TaqMan® 5′-exonuclease allelic discrimination assay. DNA was extracted using standard methods from blood collected from each individual. Genotype reproducibility was routinely assessed by regenotyping samples for selected SNPs and was generally >99%. Genotyping completion rate was, greater than 95% and genotyping errors were detected as Mendelization errors and haplotype inconsistency via MERLIN version 1.0.1, which identifies improbable recombination events from dense SNP maps.

The three intronic risk associated SNPs in the ErbB4 gene (rs7598440; rs839523; rs707284) were genotyped. Overall genotyping failure rate was less than 1%. The program SNPHAP (version 1.0, a program for estimating frequencies of haplotypes of large numbers of diallelic markers from unphased genotype data from unrelated subjects written by and freely available from David Clayton, Cambridge Institute for Medical Research) was used to calculate haplotype frequencies and to assign diplotypes to individuals. Individuals were divided according to diplotype into three groups, risk hap homozygotes (AAG/AAG); risk hap carrier (AGG/non risk) and non risk/non risk (all other diplotypes).

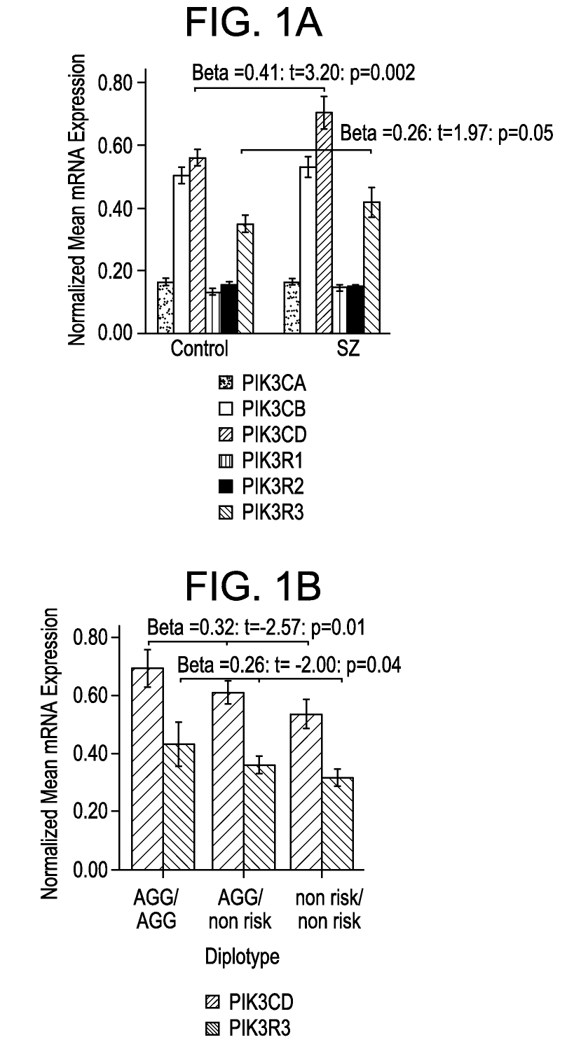

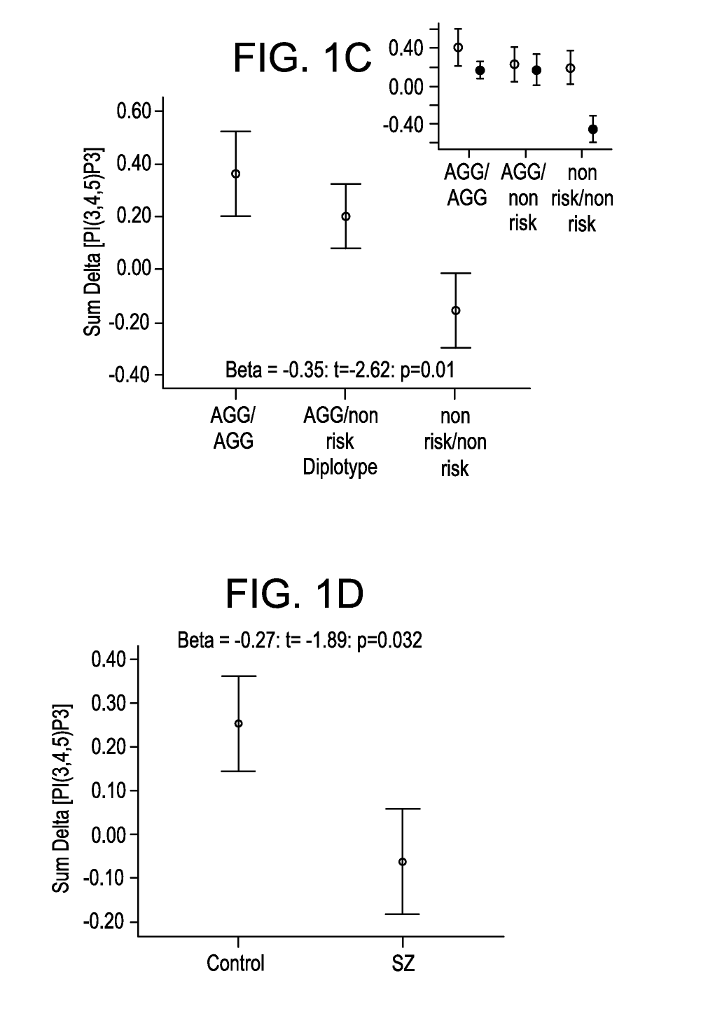

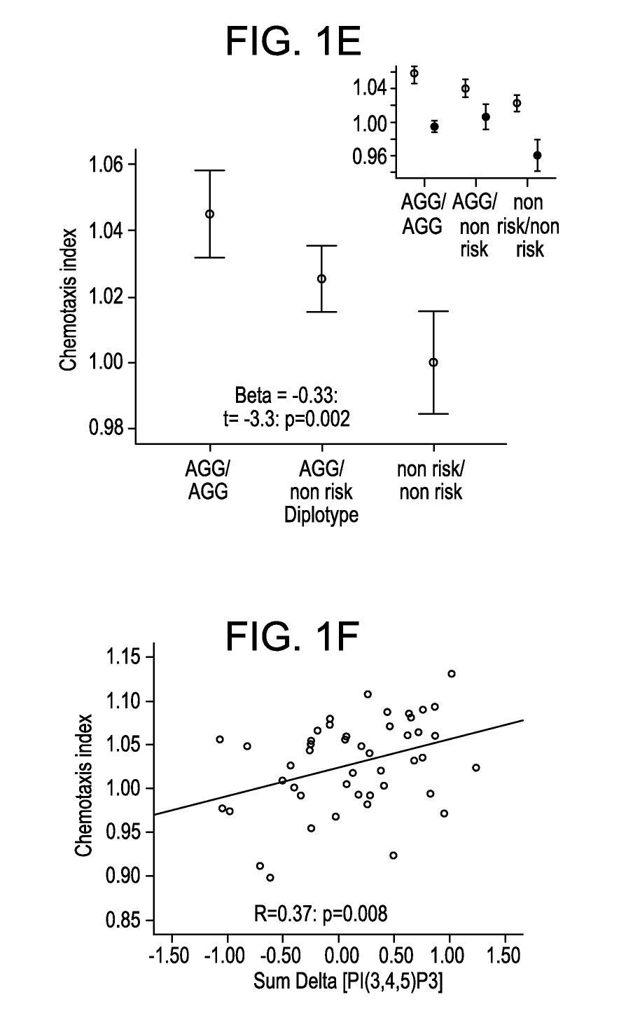

The results of the expression determinations as a function of population characteristics are shown in FIG. 1 panels (A) and (B). Expression of PIK3CD and PIK3R3 is increased in schizophrenia and is also associated with ErbB4 risk genotype.

Twenty three percent of the variance in PIK3CD expression was explained by two factors (full model F(3,52)=5.0, p=0.004): schizophrenia and ErbB4 haplotype, with a 40% increase in schizophrenia (FIG. 1A) and greater expression in subjects with the AGG risk haplotype (FIG. 1B). A similar genotype effect on PIK3R3 was seen, with 17% of the variance (full model F(3,52)=3.4, p=0.025) explained by schizophrenia (FIG. 1A) and by ErbB4 haplotype (FIG. 1B).

Analysis shows that 23% of the variance in PIK3CD expression was explained by two factors (full model F(3,52)=5.0, p=0.004): schizophrenia (P=0.41; t=3.20; p=0.002) and ErbB4 AGG risk haplotype (P=−0.32; t=−2.57; p=0.01), with a 40% increase in schizophrenia (FIG. 1A) and greater expression in subjects with the AGG risk haplotype (FIG. 1B). A similar effect on PIK3R3 expression was seen, with 17% of the variance (full model F(3,52)=3.4, p=0.025) explained by schizophrenia (P=0.26; t=1.97; p=0.05; FIG. 1A) and by the ErbB4 risk haplotype (P=−0.26; t=−2.0; p=0.04; FIG. 1B). These results demonstrate an influence of schizophrenia and ErbB4 risk genetic variation on expression traits of selected PI3K subunits in the same directionality as on expression of ErbB4, CYT-1.

To test whether these effects are specific to the PI3K pathway, expression of genes in the ErbB4-MAPK pathway (Shc, GRB2, SOS1, and MAPKI) to which ErbB4-CYT1 also couples were examined. No diagnostic or genotype effects were seen.

A positive correlation was found in human LCLs between PIK3R3 and PIK3CD transcripts (Spearman's rho=0.41, p=0.002) independent of ErbB4 genetic variation. The effect of ErbB4 genetic variation and schizophrenia on PIK3R3 expression was explained by PIK3CD alone (model F(4,51)=3.9, p=0.007; P=0.43; 3), suggesting that altered PIK3R3 expression is secondary to changes in PIK3CD. These correlational data, combined with evidence implicating PIK3CD but not PIK3R3 genetically in schizophrenia and adult brain function (see below), indicate that the primary PI3K abnormality concerns PIK3CD.

### Example 2

### NRG1 Activation of the PI3K Pathway is Deficient in Schizophrenia and Related to Disease-Associated Polymorphisms in ErbB4

PI3K catalyses formation of phosphatidylinositol-3,4,5-triphosphate ([PI(3,4,5)P3]). To address the biochemical consequences of increased PIK3CD expression, downstream of schizophrenia risk-associated variation in ErbB4, flow cytometry was used to measure NRG1 induced intracellular [P1(3,4,5)P3] production in LCLs.

Flow cytometric analysis of NRG1-stimulated [PI(3,4,5)P3] production was obtained for LCLs from 29 of the controls and 19 patients in the cohort described above in Example 1.

Intracellular staining was used to determine relative [PI(3,4,5)P3] concentrations at the single cell level using the Cytofix/Cytoperm™ kit (BD Biosciences, San Jose, Calif.). Cells were stimulated with either NRG1a (100 ng/ml) or CD 19B cell receptor (BCR) crosslinking in a 5% CO₂ incubator at 37° C. For the CD19/BCR crosslinking, cells were incubated with mouse monoclonal anti-human IgM antibody (BD Biosciences) and mouse monoclonal anti-CD 19 antibody (BD Biosciences) followed by incubation with goat anti-mouse antibody (Pierce, Rockford, Ill.). The reaction was terminated at 5, 10, 15 and 30 min by fixing cells with Phosflow Fix Buffer I (BD Bioscience) for 10 min at 37° C. Baseline represented 0 time point in the absence of NRG1α stimulation. Cells were washed with Phosflow Perm/Wash Buffer I (BD Bioscience), permeabilized in Phosflow Perm/Wash Buffer I, and stained with biotin-conjugated anti-[PI(3,4,5)P3] antibody (Echelon Biosciences Inc., Salt Lake City, Utah) for 1 hr at room temperature. After washing twice with Phosflow Perm/Wash Buffer I, cells were incubated with phycoerythrin-conjugated avidin (BD Bioscience). After washing with Phosflow Perm/Wash Buffer I, cells were analyzed using FACScan (BD Bioscience). CellQuest software (BD Bioscience) was used to acquire and quantify the fluorescence signal intensities. Data are presented as Sum Delta [PI(3,4,5)P3] calculated as the sum of ratios ([geometric mean of fluorescent intensity level (GMF)-baseline GMF]/baseline GMF) over 5 consecutive time points (0, 5, 10, 15 and 30 min). NRG1-induced [PI(3,4,5)P3] production was blocked in a dose-dependent manner by treatment with wortmannin.

The flow cytometry results are shown in FIGS. 1C and 1D. FIG. 1C shows NRG1 induced [PI(3,4,5)P3] production in LCLs graphed as a function of the number of AGG alleles of the ErbB4 risk haplotype in the whole sample (AGG/AGG, n=11; AGG/non risk, n=23, non risk-non risk n=13), with the inset showing a graph of the data parsed by diagnostic group (darker bars are patients with schizophrenia). The results of a multiple linear regression on the whole sample between NRG1 induced [PI(3,4,5)P3] production in LCLs and the number of AGG alleles of the ErbB4 risk haplotype is also shown in FIG. 1C. LCLs from subjects homozygous or heterozygous for the ErbB4 risk haplotype exhibited significantly greater [PI(3,4,5)P3] production in response to NRG1 stimulation compared to LCLs from individuals who did not carry the haplotype (FIG. 1C). This genotype effect appears in both controls and patients (FIG. 1C, inset) FIG. 1D shows NRG1-stimulated intracellular [PI(3,4,5)P3] production in controls and in patients with schizophrenia (n=29 vs. 19). LCLs from patients with schizophrenia showed a decreased response to NRG1 stimulation (P=−0.27; t=−1 29; p=0.032; FIG. 1D), independent of genotype.

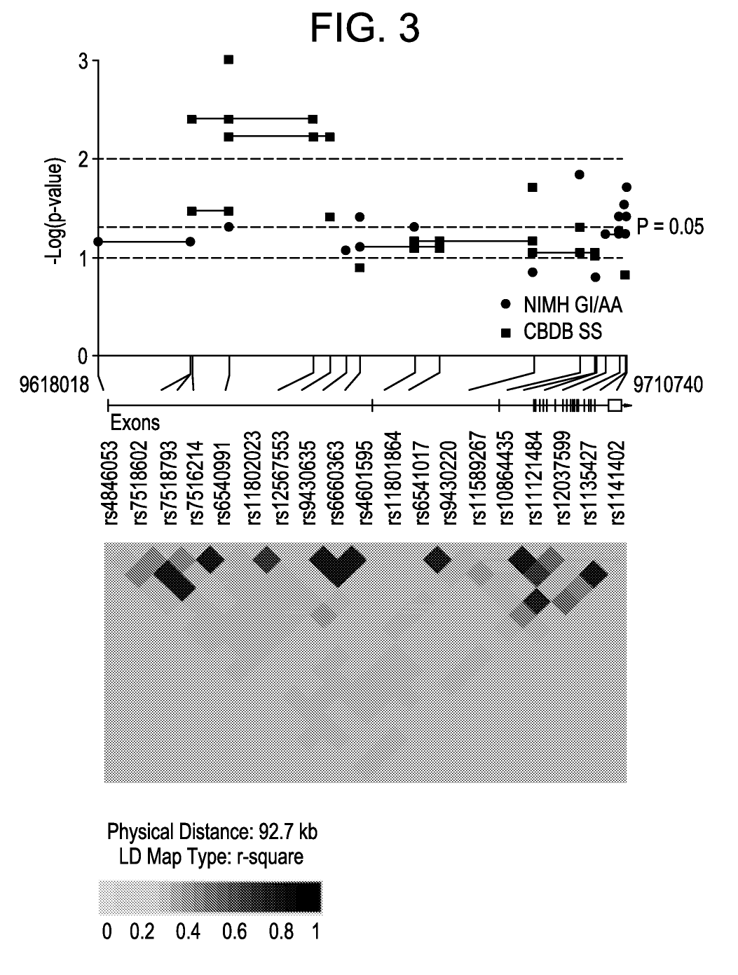

Analysis showed that 29% of the variance in NRG1 induced [PI(3,4,5)P3] production was explained by three factors (full model F(4,44)=5.19, p=0.004): 1) the ErbB4 risk haplotype (P=−0.35; t=−2.62; p=0.01; FIG. 1 C); 2) schizophrenia (P=−0.27; t=−1.89; p=0.032; FIG. 1D) and 3) PIK3CD mRNA and protein expression (mRNA, P=−0.35; t=−2.96; p=0.005; protein, P=−0.20; t=−1.49; p=0.05).

These biochemical data are indicative of blunted NRG1-mediated PI3K signaling in the disease and are consistent with the inverse relationship observed between either PIK3CD mRNA or protein expression and [P1(3,4,5)P3] production noted above.

PIK3CD also mediates B cell antigen receptor (BCR) mediated [PI(3,4,5)P3] production. If the molecular effect of the ErbB4 schizophrenia-associated haplotype is specifically on NRG1/ErbB4-stimulated PIK3CD activation, there should be no association with CDI9/BCR induced activation of PIK3CD. No such association was observed. Hence, schizophrenia and the ErbB4 risk haplotype appear related specifically to a NRG1-ErbB4-CYT-1-PI3KCD pathway, rather than there being a more general abnormality of PI3K-activating pathways.

### Example 3

### NRG1 Mediated Chemotaxis is Influenced by Intracellular [P1(3,4,5)P3] Production, Disease-Associated Polymorphisms in ErbB4, and Schizophrenia

PI3K-dependent signaling regulates cell migration, ErbB4 plays a critical role in neuronal migration, and ErbB4 CYT-I mediates PI3K dependent NRG1 induced chemotaxis. Moreover, NRG1-stimulated chemotaxis is impaired in LCLs of the patients investigated here, providing a more complex cell based phenotype for assessing NRG1-ErbB4-PI3K signaling in the disease.

Given that genetic variation in ErbB4 was observed to influence the activity of the PI3K system, NRG1-induced cell migration should be impacted.

LCL migration was analyzed using a transwell chemotaxis assay carried out using an InnoCyte™ chemotaxis chamber with an 8-mm pore size (Calbiochem) or a QCM chemotaxis chamber with a 5-mm pore size (Chemicon, Temecula, Calif., USA), according to manufacturer's instructions. Cells were suspended at 4×105 cells/mL in serum-free RPMI-1640 and 100-150 mL of the cell suspension (40,000-60,000 cells) was applied to the upper wells of the chemotaxis chamber. Serum-free RPMI-1640 with or without NRG1 (200 μL/well) was added to the lower wells as a chemotractant. After 4-24 h of incubation (95% air/5% CO₂ at 37° C.), cells attached to the lower side of the membrane were detached by detachment solution provided in the kit, lysed with 0.1% Triton X-100, and measured using CyQUANT® GR double-stranded DNA detecting reagent (Molecular Probes, Eugene, Oreg., USA). For the InnoCyte kit migration assay, migrated cells were measured following labeling cells with Calcein-AM. Chemotaxis index is defined as the ratio of migration in response to NRG1 exposure to migration in response to vehicle control. All assays were carried out in triplicate.

Results from the chemotaxis assays are shown in FIGS. 1E and F. FIG. 1E shows a graph of chemotaxis to NRG1 as a function of diplotype of the ErbB4 risk haplotype (AGG/AGG, N=12; AGG/non risk, n=28, non risk/non risk n=12) for the whole sample, with the inset showing the data parsed by diagnosis. As shown in FIG. 1E, increasing numbers of the ErbB4 risk haplotype predicted increased chemotactic response to NRG1, consistent with its effect on [PI(3,4,5)P3] production. FIG. 1E also shows that individuals null for the ErbB4 AGG risk haplotype did not migrate towards NRG1, reflecting their lack of ErbB4 expression and of [PI(3,4,5)P3] production (FIG. 1C). These data demonstrate that ErbB4 signaling is essential for mediating NRG1-induced migration in LCLs, as it is in neural progenitor cells. FIG. 1F shows a graph of chemotaxis to NRG1 as a function of NRG1-induced [PI(3,4,5)P3] production (n=47). The chemotaxis index increases linearly with [P1(3,4,5)P3] production.

In summary, 34% of the variance in NRG1-induced cell migration was explained by three factors (full model: F(4,44)=6.84, p=0.001): 1) ErbB4 haplotype (P=−0.33; t=−3.3; p=0.002; FIG. 1E), 2) [P1(3,4,5)P3] production (P=0.37; t=2.64; p=0.008; FIG. 1F) and 3) PIK3CD expression (protein/3=-0.39; t=−3.00; p=0.005; mRNA/3=−0.20; t=−1.55; p=0.05; data not shown).

The observation that NRG1 stimulated [P1(3,4,5)P3] production and cell migration are correlated and impaired in schizophrenia suggests that blunted NRG1-mediated PI3K signaling represents a pathogenic foundation for impaired cell migration in the disorder.

### Example 4

### PIK3CD Expression in Brain is Predicted by Disease-Associated Polymorphisms in ErbB4 and is Influenced by Antipsychotic Medication

In this example, the molecular phenotypes related to the ErbB4-PI3K pathway observed in human LCLs were confirmed to be similar to those in human brain cells representative of the disease state.

Postmortem brain tissue was collected at the Clinical Brain Disorders Branch, National Institute of Mental Health (NIMH). Brain tissue from 72 normal controls (22 females/50 males; 46 African American, 21 American Caucasian, 4 Hispanic, and 1 Asian individual; mean age 41.5 years±15.2 years (standard deviation (SD)), postmortem interval (PMI) 30.2±14.1 hrs, pH 6.59±0.32); and 31 schizophrenic patients (13 females/18 males; 18 African Americans, 11 Caucasians; mean age 48.5±17.7 years, PMI, 35.1±17.6 hrs, pH 6.49±0.24) was available for this study. Diagnoses were determined by independent reviews of clinical records and family interviews by two psychiatrists using DSM-IV criteria. Inpatient and outpatient clinical records were reviewed for every subject. Macro- and microscopic neuropathological examinations and toxicology screening were performed on all cases prior to inclusion in the study. The different genotype groups in this cohort did not differ on any of the measured variables that potentially affect gene expression in human postmortem brain (i.e., I, pH, and RNA Integrity Number (RIN).

Tissue from dorsolateral prefrontal cortical gray matter (DLPFC) and the hippocampus, two brain regions prominently implicated in the pathophysiology of schizophrenia, were stored at −80° C. Total RNA was extracted from 300 mg of tissue using TRIZOL® Reagent (Life Technologies Inc., Grand Island, N.Y., USA). Reverse transcription and RT-PCR were performed as described in Example 1. Genotyping of the SNPs of the ErbB4 risk haplotype in DNA extracted from the brain tissue was also performed as described in Example 1.

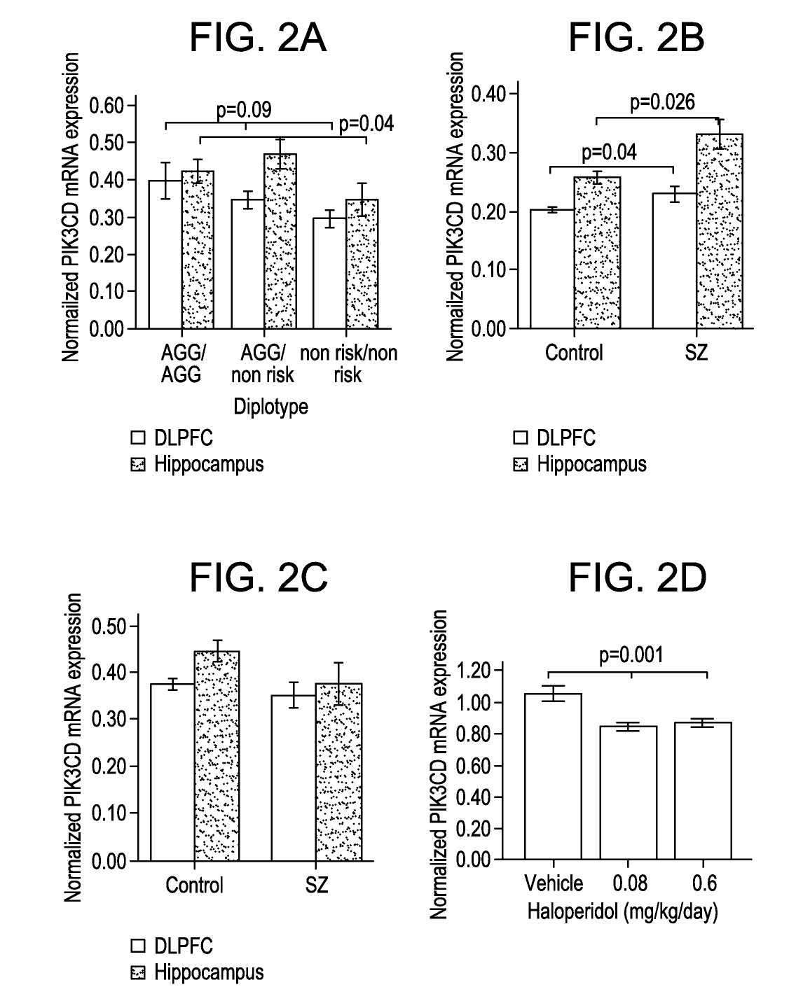

Results of the experiments using brain tissue are shown in FIG. 2. FIG. 2A shows a histogram of PIK3CD mRNA expression of normal controls as a function of diplotype of the ErbB4 risk haplotype in DLPFC and hippocampus. The association between diplotype and expression of PIK3CD in the hippocampus and DLPFC was analyzed by univariate ANOVA. FIG. 2A shows that in normal controls, presence of the ErbB4 risk haplotype (hippocampus, n=4 AGG/AGG; n=24 AGG/non risk; n=27, non risk/non risk; DLPFC, n=5 AGG/AGG; n=30 AGG/non risk; n=32, non risk/non risk) predicted increased PIK3CD mRNA expression in hippocampus (F (2, 53)=3.08; p=0.04) and in DLPFC (F(2, 65)=1.69, p=0.09). As discussed further below, effects of antipsychotic treatment precluded examination of genotype effect on PIK3CD mRNA expression in patients with schizophrenia.

FIG. 2B presents a histogram of PIK3R3 mRNA expression as a function of disease state (control or schizophrenia) in DLPFC and hippocampus. Univariate ANOVA, covaried for age, pH and PMI, indicated that PIK3R3 mRNA is increased in both brain regions in patients with schizophrenia compared to the controls, replicating the findings in LCLs of increased PIK3R3 expression in schizophrenia (FIG. 1A). Similarly, ErbB3 mRNA expression in DLPFC of patients with schizophrenia was shown to decrease compared to the controls (F (1, 105)=4.98; p=0.028), replicating the ErbB3 mRNA expression findings in LCLs.

FIG. 2C presents a histogram of PIK3CD mRNA expression as a function of disease state (control or schizophrenia) in DLPFC and hippocampus. Univariate ANOVA, covaried for age, pH, and PMI, indicated that PIK3CD mRNA is not increased in either brain region in patients with schizophrenia compared to the controls (DLPFC, n=72 controls vs. 31 SZ; hippocampus, n=69 controls vs. 31 SZ). This observation was in contrast to the increase in PIK3CD expression seen in the LCLs from patients with schizophrenia (FIG. 1A). We hypothesized that this was due to antipsychotic medications received by all the patients for extended periods prior to death and that this confound likely does not apply to the LCLs in view of their transformation and multiple passages.

To explore this hypothesis, rats treated chronically with haloperidol, a standard antipsychotic drug, were tested for PIK3CD expression in the brain. Male Sprague-Dawley rats (weight 250 g) were on a 12-h light/dark cycle (lights on/off 0600 hours/1800 hours) in a temperature-controlled environment and with access to food and water. Rats were randomly assigned to drug treatment groups (8 per dose) and administered intraperitoneal injections of haloperidol (0.08 or 0.6 mg/kg/day) or vehicle (0.02% lactic acid) once daily for 28 days. Haloperidol (20 mg/ml) was prepared in 1% lactic acid, diluted with water, and neutralized with 1M NaOH to obtain pH 5.3. Rats were euthanized 7 h after the last injection. Brains were dissected and frozen at −80° C. PIK3CD, PIK3R3, ErbB4, and ErbB3 mRNA expression was determined in rat hippocampus using methods described above. Association of mRNA expression with haloperidol treatment was analyzed by ANOVA.

Results for PIK3CD mRNA expression in rat brain as a function of haloperidol treatment are shown in FIG. 2D. Chronic administration of haloperidol to rats decreases expression of PIK3CD mRNA in rat hippocampus compared to untreated rats (F(2,22)=9.45, p=0.001). In contrast, haloperidol did not affect PIK3R3, ErbB4, or ErbB3 mRNA expression in rat hippocampus.

These findings in rat brain provide a possible explanation for the unchanged expression of PIK3CD mRNA in the brains of subjects with schizophrenia. Additionally, these findings suggest that PIK3CD is relevant to actions of antipsychotic drugs, specifically that PIK3CD could be a therapeutic target for potential new drugs for treating CNS disorders.

### Example 5

### Genetic Dissection of ErbB4 Pathways Identifies PIK3CD as a Novel Schizophrenia Susceptibility Gene

Risk genes for schizophrenia and other complex disorders appear to be clustered in specific cellular pathways. In view of the findings of molecular interaction between ErbB4 and PIK3CD disclosed above, and the prior evidence that NRG1 and ErbB4 are schizophrenia susceptibility genes, as is AKTI, a downstream target of PI3K activity, PIK3CD was investigated for clinical genetic association with schizophrenia.

An 8.2 kb region of PIK3CD (including all exons and promoters) was resequenced and 20 single nucleotide polymorphisms (SNPs) spanning a 92.72 Kb region (chr 1: 9, 618,018-9,710,740) encompassing PIK3CD (77.17 kb gene; chr 1:9,634,390-9,711,563) were genotyped. Association of the SNPs with schizophrenia was tested in two independent family samples and two case control datasets (406 families, 946 patients, and 1114 independent controls). An empirical P-value for association significance was calculated using permutation testing. The SNPs comprised 13 tag SNPs from HAPMAP (rel 22/Phase II) and 7 SNPs selected in potentially functional domains including known promoters, 5′ and 3′ untranslated regions (UTR), and conserved noncoding sequences (Table 1).

Three independent clinical samples were used for clinical genetic study. The principal family sample was ascertained as part of the CBDB/National Institute of Mental Health (NIMH) Sibling Study (SS). DNA was available from 445 probands, 400 siblings of probands, 612 parents, and 488 unrelated controls. All probands met DSM-IV criteria for a broad diagnosis category consisting of schizophrenia, schizoaffective disorder, simple schizophrenia, psychosis NOS, delusional disorder, schizotypal, schizoid, or paranoid personality disorder. Control subjects were ascertained from the NIMH normal volunteer office and required absence of diagnosis of a psychiatric disorder, extended to include first-degree relatives. For family based association analysis, families (n=356) with a single affected proband were examined. A partially-independent case-control analysis was employed comprising 445 unrelated probands and 488 unrelated healthy controls. Inclusion criteria for all participants included: self-identification as Caucasian (mostly European ancestry), aged between 18 to 60 years and IQ scores above 70 (for probands, premorbid IQ).

A second smaller independent sample for follow-up investigation to confirm family based association with schizophrenia was obtained from the NIMH Genetics Initiative (NIMH-GI) consisting of n=50 African American families (GI-AA). A different ascertainment strategy for collection was used compared to the principal CBDBNIMH cohort, being collected for linkage analysis and consisting of families with multiple affected siblings. Only nuclear families were included with DNA available from at least one sibling with a diagnosis of schizophrenia or schizoaffective disorder, and at least one parent.

A third cohort was collected from the Munich area in Germany consisting of 501 unrelated schizophrenia patients and 626 unrelated healthy controls, all self-identified Caucasian.

Clinical genetic association and epistasis analyses were conducted using logistic regression and family-based association testing using the software program, FBAT (freely available from Nan Laird, Harvard University).

Hardy Weinberg equilibrium was tested with Fisher's exact test. Linkage disequilibrium (LD) between markers was measured with the D′ and r² statistics from unrelated controls and founders in families using LDMAX within the Graphical Overview of Linkage Disequilibrium (GOLD) software package (Abecasis G R et al. (2000) Bioinformatics 16:182-3). Main effects analyses of single SNPs were conducted using unconditional logistic regression models and haplotype analysis was performed using the score statistic-based test implemented in the R package haplo.stats, controlling for sex and age in the case-control sample and using FBAT in families to test both single SNPs and haplotypes. Three-SNP haplotypes were tested for association in a sliding window across the gene with permutation testing for significance assessment.

Family-based association results are depicted in FIG. 3 and Table 1. FIG. 3, created using the R package snp.plotter (available as a contributed software package from the Comprehensive R Archive Network), shows a linear schematic representation of the PIK3CD gene region from nucleotides 9618018 to 9710740 in the center of the graphic. An arrowhead indicates the direction of transcription and the short, vertical lines indicates position of the exons. The relative positions of the 20 PIK3CD SNPs tested are also represented. Superimposed on the PIK3CD gene are test results for single SNPs, sliding window 3 SNP haplotypes, and linkage disequilibrium (LD) over PIK3CD in the CBDB SS sample and NIMHGI-AA family cohorts. In the plot of -log(p-value) vs. chromosome map position above the PIK3CD gene, the horizontal dashed lines are at p=0.1, p=0.05 and p=0.01. Below the PIK3CD gene, linkage disequilibrium (LD), expressed as r², between SNP loci is indicated for 370 unrelated healthy Caucasian controls.

TABLE 1
PIK3CD Single-marker association results for CBDB family based, NIMHGI-AA, and CBDB case-control cohorts

| | | | | | CBDB SS Study | | | | NIMHGI-AA | | | | Case-Control | | | | | |
|---|---|---|---|---|---|---|---|---|---|---|---|---|---|---|---|---|---|---|
| | Location | | Posi- | | 356 families | | | | 50 families | | | | 445 patients and 448 controls | | | | | |
| | bp UCSC | SEQ | tion in | | | Emp | | | | Emp | | | MAF | | | | | |
| | Build 36 | ID | SEQ | Al- | | p | | | | p | | | con- | MAF | Geno- | | (95% | P |
| SNP rs | (HG18) | NO | ID NO | leles | MAF | value | Assoc | Risk | MAF | value | Assoc | Risk | trols | cases | type | OR | CI) | value |
| rs4846053 | 9618018 | — | | G/C | 0.24 | 0.39 | | | 0.53 | 0.39 | | | 0.25 | 0.25 | | | | 0.76 |
| rs7518602 | 9633931 | 1 | 1418 | C/T | 0.38 | 0.20 | | | 0.85 | 0.37 | | | 0.41 | 0.39 | | | | 0.17 |
| rs7518793 | 9634074 | 2 | 976 | C/T | 0.21 | 0.59 | | | 0.29 | 0.63 | | | 0.2 | 0.21 | | | | 0.79 |
| rs7516214 | 9634324 | — | | A/G | 0.37 | 0.66 | | | 0.65 | 0.55 | | | 0.41 | 0.38 | | | | 0.16 |
| rs6540991 | 9640674 | 3 | 201 | T/C | 0.31 | 0.05 | + | T | 0.72 | 0.001 | + | T | 0.34 | 0.32 | T/C | 0.7 | (0.51- | 0.02 |
| | | | | | | | | | | | | | | | | | 0.96) | |
| rs11802023 | 9655498 | — | | C/T | 0.08 | 0.50 | | | 0.09 | 0.32 | | | 0.09 | 0.08 | | | | 0.28 |
| rs12567553 | 9658431 | 4 | 501 | A/G | 0.12 | 0.66 | | | 0.56 | 0.04 | + | A | 0.15 | 0.13 | | | | 0.2 |
| rs9430635 | 9661373 | 5 | 251 | C/G | 0.48 | 0.07 | | | 0.47 | 0.851 | | | 0.46 | 0.46 | | | | 0.16 |
| rs6660363 | 9663780 | 6 | 1437 | A/G | 0.48 | 0.04 | + | A | 0.31 | 0.127 | | | 0.46 | 0.47 | | | | 0.36 |
| rs4601595 | 9673413 | 7 | 301 | G/T | 0.49 | 0.05 | + | G | 0.29 | 0.08 | | | 0.45 | 0.47 | | | | 0.73 |
| rs11801864 | 9677860 | 8 | 501 | G/A | 0.04 | 0.26 | | | 0.23 | 0.08 | | | 0.04 | 0.04 | | | | 0.9 |
| rs6541017 | 9694151 | 9 | 900 | A/G | 0.17 | 0.14 | | | 0.18 | 0.02 | + | A | 0.15 | 0.15 | | | | 0.73 |
| rs9430220 | 9702458 | 10 | 401 | T/C | 0.24 | 0.01 | + | T | 0.6 | 0.05 | + | T | 0.24 | 0.21 | C/C | 0.46 | (0.22- | 0.03 |
| | | | | | | | | | | | | | | | | | 0.94) | |
| rs11589267 | 9705143 | 11 | 401 | C/T | 0.45 | 0.16 | | | 0.11 | 0.1 | | | 0.46 | 0.45 | C/T | 1.48 | (1.05- | 0.02 |
| | | | | | | | | | | | | | | | | | 2.07) | |
| rs10864435 | 9705353 | — | | C/T | 0.08 | 0.48 | | | 0.33 | 0.976 | | | 0.09 | 0.08 | | | | 0.28 |
| rs11121484 | 9707010 | — | | C/T | 0.11 | 0.21 | | | 0.47 | 0.97 | | | 0.1 | 0.11 | | | | 0.99 |
| rs12037599 | 9709432 | 12 | 401 | G/C | 0.04 | 0.05 | + | G | 0.28 | 0.791 | | | 0.05 | 0.04 | | | | 0.29 |
| rs1135427 | 9710427 | 13 | 401 | T/G | 0.45 | 0.03 | + | T | 0.75 | 0.154 | | | 0.44 | 0.45 | T/G | 1.46 | (1.04- | 0.02 |
| | | | | | | | | | | | | | | | | | 2.04) | |
| rs1141402 | 9710740 | 14 | 201 | G/A | 0.05 | 0.02 | + | G | 0.25 | 0.871 | | | 0.05 | 0.04 | | | | 0.48 |

CBDB SS Clinical Brain Disorders Branch Sibling study families,
NIMHGI-AA NIMH genetics initiative African American families.
Alleles presented as major/minor alleles in the CEPH population (Utah residents with ancestry from northern and western Europe) (abbreviation: CEU) of HapMapPhase III (rel. 1) (The International HapMap Consortium. The International HapMap Project. Nature 426, 789-796 (2003)).
Minor allele frequency (MAF) set in SS Caucasian controls. MAF in family samples stated from parents.
Association risk (assoc) = positive (+) when 1 allele over transmitted.
Bold denotes consistent replication and directionality across all three study samples.
The empirical P-value for association significance was calculated using permutation testing.
Case control dataset representative of 445 probands and 488 healthy controls

Single SNP analysis in the CBDB sibling study (CBDB SS) families revealed nominal evidence for association with schizophrenia to SNPs in the PIK3CD 5′ promoter region, (rs6540991, p=0.05; rs6660363, p=0.04 and rs4601595, p=0.05); the 3′ intronic region (rs9430220, p=0.01) and the 3′ UTR (rs1141402, p=0.02; rs1135427, p=0.03 and rs12037599, p=0.05) (See Table I). The 5′ and 3′ markers showing association are in weak LD with each other (D′ range: 0.26 to 0.41; r² range: 0.04 to 0.1), suggesting at least two independent signals within the gene. Sliding window 3-SNP haplotypes containing these SNPs were also significantly associated but the haplotypic p-values were not smaller than the individual markers (FIG. 3).

Only one SNP at p<0.05 would have been expected by chance. Furthermore, association of three of the above SNPs, and to the same alleles, was replicated in a dataset comparing unrelated cases from the SS family data (plus 100 additional cases) to a set of independent unrelated controls: rs6540991 (p=0.02); rs9430220 (p=0.03) and rs1135427 (p=0.02) (See Table 1). In the NIMHGI African-American family sample, significant association to the same allele was again replicated for rs6540991 (p=0.001) and rs9430220 (p=0.05), with suggestive evidence for rs4601595 (p=0.08), and a novel association to another 5′ SNP, rs12567553 (p=0.04; Table 1). No significant association to any SNP was observed in the German case control sample. These findings provide evidence for association of PIK3CD with risk for schizophrenia in Caucasian and African American individuals.

Subsequently, 25 additional SNPs in PIK3R3 and 9 SNPs in ErbB3 were evaluated in the CBDB SS samples for association with schizophrenia. No association of any of these SNPs with schizophrenia was found (all p>0.2), supporting the interpretation that alterations in PIK3R3 and ErbB3 expression in schizophrenia are secondary or compensatory to a cardinal PIK3CD and ErbB4 involvement in the disorder that includes a role in its genetic risk architecture.

### Example 6

### Genetic Interaction Between ErbB4 and PIK3CD Further Increases Risk for Schizophrenia

Epistasis is recognized as fundamentally important to understanding the structure and function of genetic pathways and the complex genetic basis of many common medical disorders. Given the evidence that interactions between ErbB4 and PIK3CD exist at the molecular level, and PIK3CD contributes to schizophrenia risk beyond its association with ErbB4, statistical epistasis between ErbB4 and PIK3CD was examined.

To test for epistasis in the case control sample (CBDB/NIMH SS sample), logistic regression and likelihood ratio tests (LRT) comparing nested logistic regression models were used; the reduced model contained main effects, whereas the full model contained interaction terms. Tests of interaction correspond to testing whether the regression coefficients that represent interaction terms in the mathematical model equal zero or not. This approach is a standard approach for detecting gene-gene interactions in complex disease.

Likelihood ratio tests comparing nested conditional logistic regression models, in which the “cases” are the combination of alleles/genotypes transmitted to the probands and the “pseudocontrols” are those that could have been but were not transmitted to the probands, were conducted for family-based data to assess epistasis between genotypes at ErbB4 and PIK3CD and affection status. This case-pseudocontrol approach is a standard approach for testing epistasis in family-based studies. The uncorrected alpha level to determine ‘significant’ interaction was set at 0.05. A total of 19 SNPs from PIK3CD and 9 SNPs from ErbB4 (3 SNP haplotype plus sequencing SNPs) were included in the analysis.

The clinical epistasis results for combinations of ErbB4 and PIK3CD SNPs showing a significant LRT p-value are summarized in Table 2.

TABLE 2
ErbB4-PIK3CD Clinical Epistasis Results

| | ErbB4 | Position | | | | | | | | |
|---|---|---|---|---|---|---|---|---|---|---|
| ErbB4 | SEQ | in SEQ | PIK3CD | | ErbB4 | PIK3CD | | 95% CI | OR | LRT |
| SNP | ID NO | ID NO. | SNP | Sample | Genotype | Genotype | OR | for OR | P-value | P-value |
| rs7598440 | 15 | 301 | rs6541017 | SS C-C^{a} | (G/G) | (G/G) | 1.69 | (0.88-3.24) | 0.12 | 0.018 |
| rs7598440 | 15 | 301 | rs4601595 | SS C-C | (A/A) | (G/G) | 2.52 | (1.13, 5.60) | 0.023 | 0.05 |
| rs7598440 | 15 | 301 | rs4601595 | SS C-C | (A/A) | (G/T) | 0.59 | (0.34, 1.04) | 0.069 | 0.05 |
| rs839541 | 16 | 401 | rs12037599 | SS C-C | (T/T) | (G/G) | 2.55 | (1.16-5.62) | 0.020 | 0.037 |
| rs707284 | 17 | 559 | rs4601595 | SS C-C | (A/A) | (T/T) | 9.27 | (1.08, 79.29) | 0.042 | 0.030 |
| rs839539 | 18 | 451 | rs7518793 | SS fam | (G) carrier | (T) carrier | 2.62 | (1.13-6.09) | 0.025 | 0.0019 |
| rs839539 | 18 | 451 | rs11801864 | SS fam | (G) carrier | (A) carrier | 6.36 | (1.26-32.11) | 0.025 | 0.022 |
| rs1098059 | 19 | 1773 | rs12567553 | SS fam | (T) carrier | (G) carrier | 2.55 | (1.05-6.19) | 0.039 | 0.044 |
| rs1098059 | 19 | 1773 | rs11801864 | SS fam | (T) carrier | (A) carrier | 6.21 | (1.23-31.32) | 0.027 | 0.017 |
| rs839523 | 20 | 301 | rs11589267 | SS fam | (G/G) | (C/C) | 0.57 | (0.31-1.05) | 0.072 | 0.017 |
| rs62185768 | 21 | 251 | rs9430635 | SS fam | (C/C) | (C/C) | 1.97 | (1.08-3.61) | 0.028 | 0.042 |
| rs62185768 | 21 | 251 | rs6660363 | SS fam | (C/C) | (A/A) | 2.13 | (1.16-3.92) | 0.015 | 0.046 |
| rs707284 | 17 | 559 | rs9430635 | SS fam | (G/G) | (C/C) | 1.75 | (0.96-3.15) | 0.064 | 0.047 |
| rs707284 | 17 | 559 | rs7518602 | SS fam | (G/G) | (1/1) | 1.61 | (0.92-2.8) | 0.094 | 0.027 |
| rs707284 | 17 | 559 | rs6660363 | SS fam | (G/G) | (G/G) | 1.66 | (0.91-3.02) | 0.095 | 0.024 |

^{a}SS C-C Sibling study case-control, SS fam, sibling study families;
1 = major allele.

The strongest evidence for epistasis was found in the CBDB/NIMH SS sample between rs839539 in ErbB4 and rs7518793 in PIK3CD: probands carrying at least one minor allele (G) at rs839539 were preferentially transmitted the minor allele (T) of rs75 18793 (LRT p-value=0.001 9, OR=2.62, 95% CI (1.13-6.09)).

A more complex epistatic pattern was seen between ErbB4 rs7598440, a SNP in the ErbB4 risk haplotype, and two SNPs in PIK3CD (rs6541017 and rs4601595) in the CBDB case-control sample. Individuals homozygous for the major risk allele at ErbB4 rs7598440 (A/A) showed increased risk for schizophrenia when homozygous for the major allele at rs4601595 (LRT p-value=0.05, OR=2.52, 95% CI (1.13-5.60)). In contrast, individuals homozygous for the same major risk allele at ErbB4 rs7598440 (A/A) who carry one copy of the minor allele at PIK3CD rs4601595 showed ‘decreased’ risk for schizophrenia (OR=0.60, 95% CI (0.34-1.04)). These observations, which seem counterintuitive at first, demonstrate a classic ‘yin-yang’ type epistatic effect whereby epistasis can block one allelic effect (i.e. rs7598440's association with increased risk) by an allele at another locus (i.e. minor allele at rs4601595). This finding provides a plausible explanation for the lack of clinical association of rs7598440 with schizophrenia in the sample because the risk alleles at rs7598440 can be observed as ‘risk’ or ‘protective’ dependant upon genetic background at PIK3CD and thus, masking a main effect.

Overall, these results provide statistical support for the existence of genetic interactions between ErbB4 and PIK3CD, relevant to schizophrenia risk, which complement the molecular evidence that they are biologically interrelated.

### Example 7

### PIK3CD Polymorphisms Influence Cognition and Brain Physiology in Healthy Controls

If PIK3CD influences risk for schizophrenia, it presumably does this by affecting the function of the brain. Yet, direct evidence of PI3K involvement in human brain function has been absent, although there are animal studies showing that PI3K signaling has a key role in learning and memory, and that activation of PI3K signaling and accumulation of [PI(3,4,5)P3] influences neurodevelopment.

The observation that PIK3CD gene expression is robustly identified in adult human brain, consistently associated with ErbB4 risk genetic variation (FIGS. 1; 2A, B), and is modified by antipsychotic treatment in the rat brain (FIG. 2D); combined with the observation that PIK3CD shows genetic association to schizophrenia in three datasets, led us to examine whether PIK3CD SNPs also impact cognition and brain activity. Cognitive deficits, particularly those affecting working memory and executive cognition, are a core feature of schizophrenia and are also seen in unaffected monozygotic co-twins and other relatives indicating their intimate relationship to the genetic basis of the syndrome.

Normal control subjects (N=413) performed a neurocognitive battery of neuropsychological tests selected for evidence of heritability and association with risk for schizophrenia. The neurocognitive battery comprised neuropsychological tests with evidence of heritability and association with risk for schizophrenia. It included the Wechsler Memory Scale-Revised (WMS-R), Wechsler Adult Intelligence Scale-Revised (WAIS-R: arithmetic, similarities, digit-symbol-substitution and picture completion), Trailmaking Test Parts A and B, Verbal and Category Fluency, Continuous Performance Test (CPT), N-Back task, California Verbal Learning Test (CVLT), Judgment of Line Orientation, and the Wisconsin Card Sorting Test (WCST). These 24 sub-tests were reducible via principal components and confirmatory factor analyses to a 7-factor solution. These factors were more independent than the individual sub-test scores, and putatively closer to the individuals' underlying psychometric structure than any single sub-test. Factor 1 was loaded with verbal episodic memory measures from the WMS-R and CVLT; factor 2 with aspects of working memory from the N-back task; factor 3 with spatial episodic memory measures from WMS-R and Judgment of Line Orientation; factor 4 with executive cognitive control and processing speed measures from WAIS-R, trails A and B, and letter and category fluency; factor 5 with logical reasoning measures from the WCST, factor 6 with attention measures from the CPT, and factor 7 with measures from the WMS-R digit span backwards and forwards.

PIK3CD genetic contribution to these cognitive factors was analyzed in a group of 413 healthy individuals for whom neuropsychological data was available. Individual SNP association was performed via a linear regression model, controlling for age and sex, to identify variation associated with cognitive factor scores.

The prediction would be that normal individuals carrying risk-associated genotypes would show patterns of cognition more similar to patients with schizophrenia than individuals lacking such genotypes. Indeed, normal individuals carrying the risk allele at rs9430220, which is over-transmitted to patients with schizophrenia, performed poorer on tasks measuring executive function (Table 3).

TABLE 3
PIK3CD Genotype Effects on Cognitive Factor Scores

| | | | | | | | Reference |
|---|---|---|---|---|---|---|---|
| | Cognitive | | β; median (p-value) | | | | group |
| rs number | Trait | Factor | 1/2 | 2/2 | 2 carrier | Model | median |
| rs9430635 | Verbal | 1 (n = 274) | — | — | −5.05; 0.14 | β with respect to 1/1 | 0.46 |
| | memory | | | | (0.02) | | |
| rs9430635 | Digit span | 7 (n = 292) | −1.2; 0.23 | −1.89; 0 | — | β with respect to 1/1 | 0.485 |
| | | | (0.06) | (0.02) | | | |
| rs9430635 | Processing | 4 (n = 413) | — | −0.21; | — | β with respect to 1 carrier | 0.03 |
| | speed | | | −0.14 | | | |
| | | | | (0.002) | | | |
| rs11801864 | Visual | 3 (n = 272) | — | — | −1.45; 0.10 | β with respect to 1/1 | 0.23 |
| | memory | | | | (0.072) | | |
| rs11801864 | Attention | 6 (n = 388) | — | — | −0.73; 0.10 | β with respect to 1/1 | 0.18 |
| | | | | | (0.022) | | |
| rs6541017 | Processing | 4 (n = 413) | — | 0.36; 0.36 | — | β with respect to 1 carrier | −0.01 |
| | speed | | | (0.019) | | | |
| rs9430220 | Card sort | 5 (n = 380) | — | 2.66; 0.39 | — | β with respect to 1 carrier | 0.14 |
| | | | | (0.04) | | | |
| rs11802023 | Attention | 6 (n = 388) | — | — | 0.568; 0.14 | β with respect to 1/1 | 0.18 |
| | | | | | (0.022) | | |
| rs11802023 | Digit span | 7 (n = 292) | — | — | −1.41; 0 | β with respect to 1/1 | 0.36 |
| | | | | | (0.048) | | |
| rs12567553 | Digit span | 7 (n = 292) | — | — | 1.38; | β with respect to 1/1 | 0.415 |
| | | | | | −0.025 | | |
| | | | | | (0.02) | | |

Markers rs10864435, rs11121484 and rs12037599 which are in moderate LD with rs9430220 also showed association with card sort, with the 2/2 genotype (non-risk) being associated with better performance (p = 0.0001 for each genotype). Two SNPs in tight LD with rs9430635 (rs6660363 and rs4601595) also showed association to task performance digit span performance. For nucleotide designation for 1 (major) and 2 (minor) alleles refer to Table 1.

Haplotype analysis revealed that protective alleles at rs6540991 which are under-transmitted to patients with schizophrenia were found on the background of 3-SNP haplotypes that were consistently associated with better performance in both controls and patients with schizophrenia on tests of verbal memory and executive function (verbal memory; Global controls, p0.01; haplotypic p=0.01; Z, 2.4; Global patients, p=0.00007, haplotypic p value p=0.0018, Z, 3.11. N back; Global controls, p=0.05; haplotypic, p=0.003, Z, 2.88).

Association was further observed between cognitive performance and 4 SNPs that were not observed to be single-point associated with schizophrenia, an observation possibly consistent with greater penetrance of gene effects at the level of brain function than behavior. The SNP rs9430635, that showed evidence of statistical epistasis with ErbB4, showed association to the most number of cognitive traits. Overall, these findings suggest a novel role for PIK3CD in cognitive function, particularly implicating the prefrontal cortex, and provide evidence that genetic variation in PIK3CD associated with schizophrenia is associated with cognitive functions impaired in the disease.

It was next predicted that SNPs in PIK3CD and ErbB4 associated with prefrontal-linked cognition would also be linked to relevant measures of brain physiology in healthy individuals as assayed with functional magnetic resonance imaging (fMRI) during performance of the N-back working memory task, a paradigm that robustly engages the prefrontal cortex. Because abnormal behavior results from abnormal brain function, it is rational that genetic association with cognition should show even more apparent effects at the level of how the brain processes cognitive information. This so-called ‘imaging genetics approach’ has been substantiated in a number of recent reports, including the demonstration of association of a genome-wide supported psychosis variant with altered brain function in normal individuals, and is highly robust to false positive findings.

To limit multiple testing, only two SNPs, rs9430635 in PIK3CD, selected for its association to multiple aspects of prefrontal cortex related cognition, and rs7598440 in ErbB4, a SNP in the risk haplotype, were examined.

All subjects who participated in the imaging study were of European ancestry and free of any lifetime history of neurological or psychiatric illness, substance abuse problems, other medical problems, neuropsychiatric treatment, or medical treatment relevant to cerebral metabolism and blood flow. There was no significant difference in age, IQ score (WAIS-R) across the genotype groups. Gender distribution differed across genotypes for the PIK3CD SNP rs9430635 (χ²=6.227, df=2, p=0.04), but not for ErbB4 rs7598440.

During fMRI scanning, subjects performed an N-back working memory (WM) task, previously described to robustly engage dorsolateral prefrontal cortex (DLPFC) response. Briefly, N-back refers to the number of previous stimuli that the subject had to recall. The stimuli presented in all the conditions consisted of numbers (1-4) pseudorandomly displayed at the corners of a diamond-shaped box for 500 msec with an interstimulus interval of 1500 msec. A non-memory guided control condition (O-back) that required subjects to identify the stimulus currently seen, alternated with the WM condition. The WM condition required the recollection of a stimulus seen two stimuli (2-back) previously while continuing to encode new incoming stimuli. Four blocks of control condition alternated with four blocks of WM condition, for total task duration of 240 seconds. Visual stimuli were presented via a back-projection screen, and performance data were recorded thorough the use of a fiber-optic response system as the number of correct responses (accuracy) and reaction time (RT).

Each subject was scanned on a GE Signa (Milwaukee, Wis.) 3T scanner. Whole-brain gradient echo blood oxygen level-dependent (BOLD)-EPI pulse sequence was used to acquire one hundred and twenty images per run. Each functional image consisted of 24 6-mm-thick axial slices covering the entire cerebrum and most of the cerebellum (TR=2000 ms; TE=30 ms; Field of View=24 cm; Flip angle=90). Data were pre-processed and analyzed using Statistical Parametrical Mapping (SPM 5, Wellcome Department of Cognitive Neurology, London, UK). The first four volumes were discarded in order to allow for T1 equilibration effects. All functional volumes were realigned to the first volume acquired using INRlalign—a motion correction algorithm unbiased by local signal changes. Images were then spatially normalized to the Montreal Neurological Institute standard brain in the space of Talairach and Tournoux to allow group analysis. Smoothing was carried out with an 8-mm full width at half maximum isotropic three-dimensional Gaussian kernel to control for residual intersubject differences and to increase the signal-to-noise ratio. All data were screened for motion and scanner artifacts. The data were then temporally highpass-filtered with a cut-off frequency of 1/120 Hz to remove the effects of scanner signal drifts. For each experimental condition, a boxcar model convolved with the hemodynamic response function at each voxel was modeled. Subject-specific movement parameters obtained from the realignment procedure were included in the general linear model as covariates, taking into account the effects of subject motion. In the first level analyses, linear contrasts were computed producing t-statistical parameter maps at each voxel for the working memory relative to the control condition. Each contrast of interest was entered into second-level random effects analyses to identify the effect of the WM task as well as genotype group differences in BOLD responses to the task. One sample t-tests across all the subjects were carried out for each SNP and showed the general effect of WM task on brain activation irrespective of genotype. ANOVA analysis was used to compare working memory related neural activity (2-back-0-back) across ErbB4 rs7598440 genotype groups, with subjects as a random effect variable and genotype groups as the independent variable. Given the gender distribution difference in the PIK3CD rs9430635 genotype groups, an ANCOVA analysis with subjects as a random effect variable, genotype groups as the independent variable, and gender as a covariate was performed.

A hypothesis-driven region of interest (ROI) approach was used to investigate genotype related effects on functional activity in DLPFC regions that were significantly activated by the task. All second level analyses were thresholded using a significance of p=0.05 family wise corrected (FWE). Small volume correction was also applied according to the Random Field Theory within the DLPFC for the ROI analyses. Anatomical dorsolateral prefrontal cortex ROI was created using the WFU Pick atlas software (available from Wake Forest University School of Medicine, Advanced Neuroscience Imaging Research Laboratory, Winston-Salem, N.C.). Mean BOLD signal change from the peak of significant DLPFC clusters was then extracted using the Marsbar toolbox. Post-hoc tests on the mean BOLD signal change were performed using Fisher's least significant difference test (LSD). It has recently been shown that this statistical approach to genetic association with functional MRI is strongly resistant to false positives, with an overall study false-positive rate significantly less than 5% per genetic variant tested despite the apparent large number of brain voxels examined. Behavioral Accuracy and reaction time differences were analyzed using a one-way ANOVA and ANCOVA with gender as a nuisance variable for ErbB4 rs7598440 and PIK3CD rs9430635, respectively.

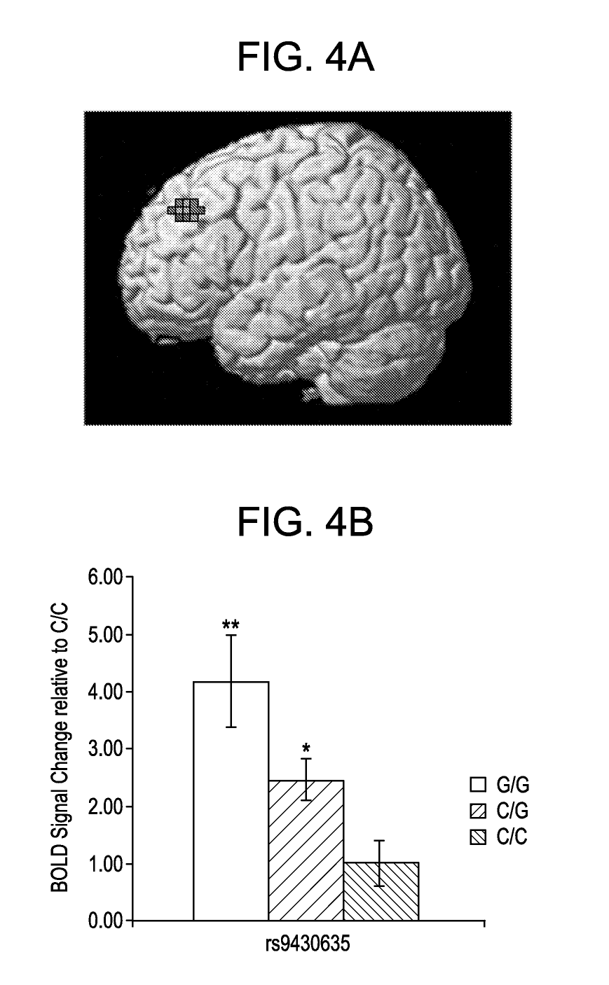

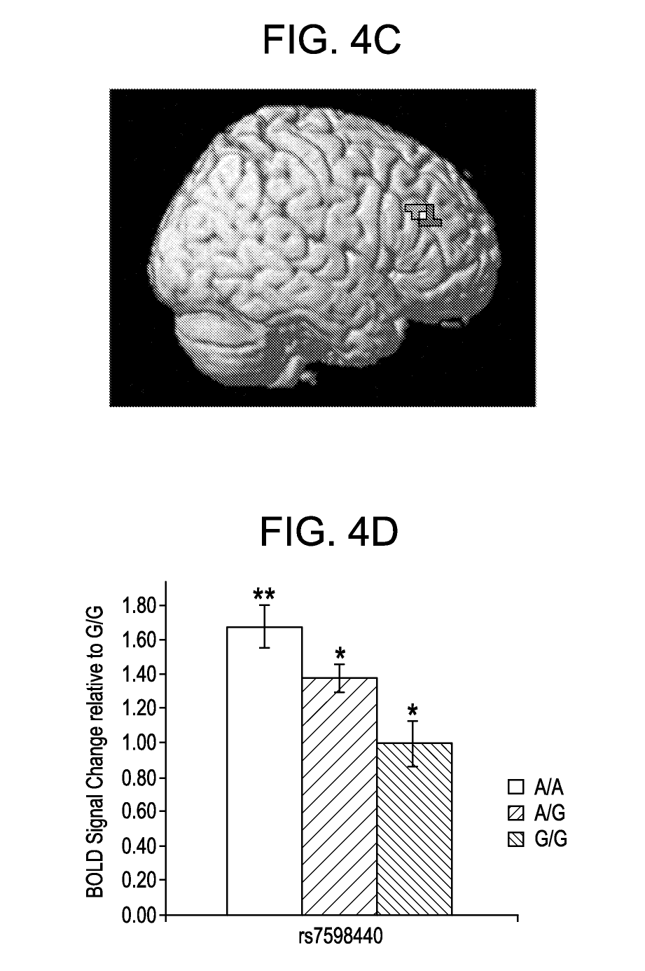

In the working memory condition, greater activation in the left DLFPC was seen in normal subjects homozygous for the minor allele (G/G) at rs9430635 (FIGS. 4A and B). GG homozygote individuals, (n=59) showed greater signal change relative to CG (n=154; post hoc Fisher's LSD, p=0.02) and CC individuals (n=82; post hoc Fisher's LSD p=0.0001).

During the same task, greater activation was observed in the right DLPFC in subjects homozygous for the clinical risk allele (A/A) at rs7598440 (FIG. 4). Individuals homozygous for the risk allele (AA, n=62) show greater signal change relative to AG subjects (n=160; post hoc Fisher's LSD p=0.06) and GG homozygotes (n=75; post hoc Fisher's LSD p=0.0001).

Since these genotype comparisons involved groups matched for performance accuracy and reaction time, the results reflect differences in brain physiology related to information processing and not to effects of test performance. This pattern of increased activation for the same level of behavioral output has been referred to as “inefficient” or “untuned” processing and has been observed during the N back task for a number of other putative schizophrenia associated genes, including AKT1, a target of PI3K function.

### Example 8

### A Genetic Variant in the PIK3CD Gene Predicts Increased Levels of PIK3CD Protein in Peripheral LCLs and Impaired NRG1-Induced Cell Migration in Individuals with Schizophrenia

A genetic variant in the PIK3CD gene, rs6540991, is over-transmitted to patients with schizophrenia. The effect of this genetic variant in the PIK3CD gene on expression of PIK3CD protein in peripheral LCLs and NRG1-induced cell migration was investigated in individuals with schizophrenia.

Genotyping of rs6540991 was performed using a commercially available TaqMan® assay, as described above in Example 1. NRG1-induced cell migration was determined using the transwell chemotaxis assay as described above in Example 2.

PIK3CD protein expression was quantified by Western Blot analysis. B lymphoblasts were lysed in TNESV buffer (50 mM Tris-HCl PH 7.4, 100 mM NaCl, 1% NP-40, 2 mM EDTA, 1 mM Na₃VO₄, and protease inhibitor cocktail) and incubated for 20 min on ice. Following centrifugation at 14000 g for 10 min, the supernatants were collected. 50 μg of protein was denatured in 4×NuPAGELDS sample buffer at 95° C. for 5 min. Samples were separated by gel electrophoresis using NuPAGE 10% bis-Tris gels and transferred to nitrocellulose membranes, then probed with the primary antibodies: 1:200 of PI3K p110d (Abcam Inc, ab32401) at 4° C. overnight; 1:10000 of anti-B-actin-HRP (Sigma, A3854) as internal control at room temperature for 1 hr and then incubated for 1 hr with 1:2000 goat anti-rabbit IgG-HRP (Santa Cruz Biotechnology, sc-2004). Protein bands were detected by ECL Western blotting analysis system (Amersham Biosciences, RPN2109) and exposed to Kodak scientific imaging film. Protein bands were imaged and the relative optical density of each band was measured using NIH Image software.

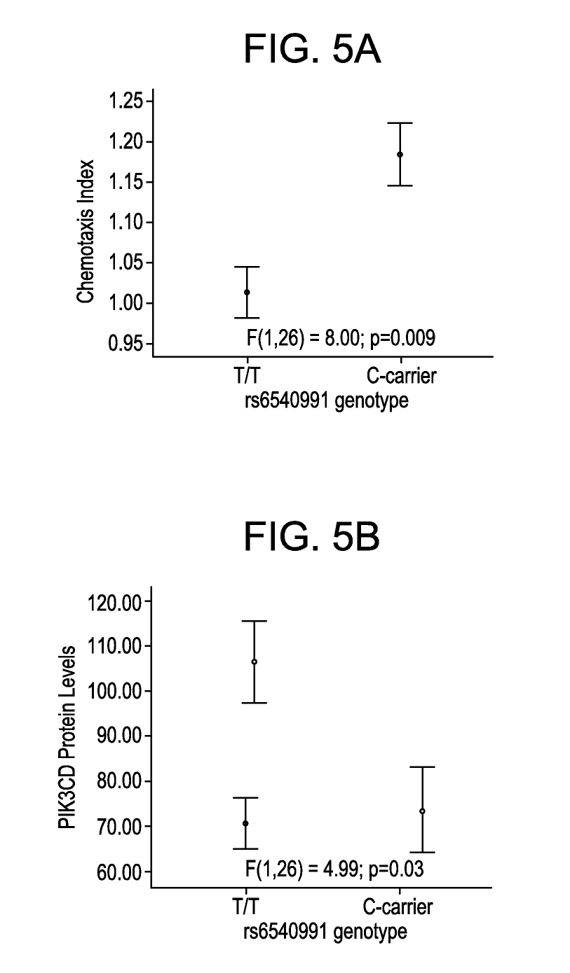

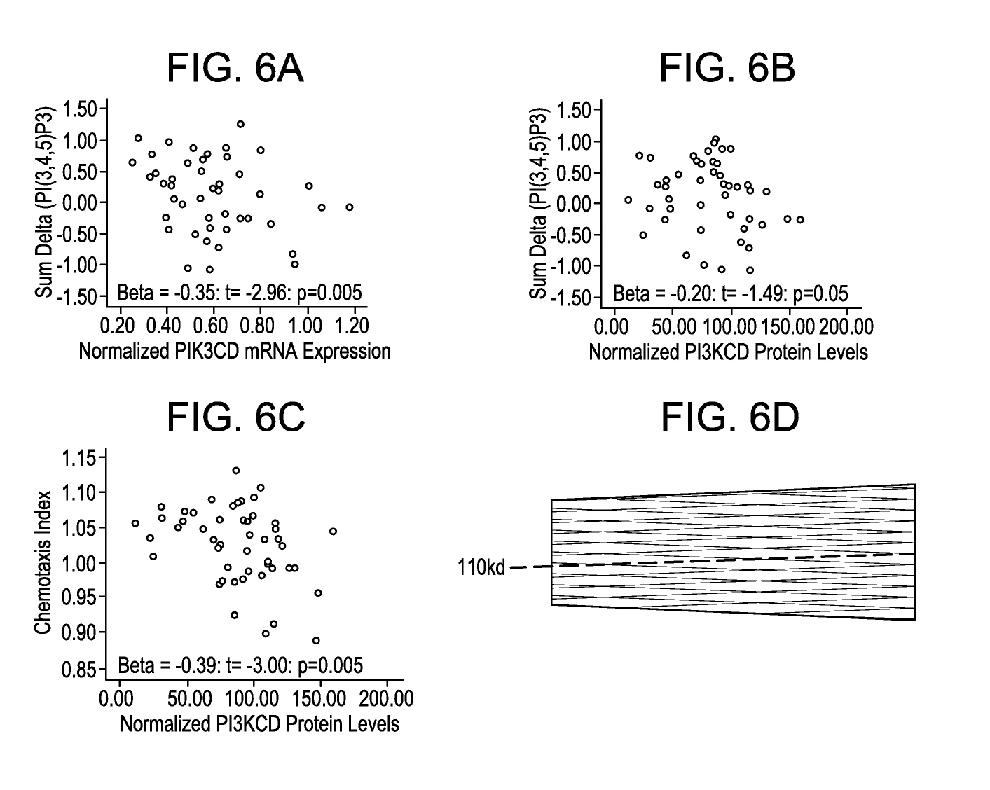

Results of these experiments are shown in FIGS. 5A and B. The T allele of rs6540991 is associated with increased levels of PIK3CD protein in peripheral LCLs (FIG. 5B) and with impaired NRG1-induced cell migration in individuals with schizophrenia (FIG. 5A). These findings are consistent with the significant inverse linear relationship observed between PIK3CD protein expression and NRG1 stimulated [PI(3,4,5)P3] production (p=0.05) (FIG. 6B) and between PIK3CD protein expression and cell migration (p=0.0005) (FIG. 6A).

Further, these findings provide a potential biological mechanism responsible for the clinical association of this region of the PIK3CD gene to schizophrenia. Specifically, these data provide evidence that in addition to the ErbB4 genotype, the therapeutic value of PIK3CD targeted compounds, such as IC87114, can be predicted by PIK3CD genotype. Moreover, peripheral and CNS ErbB4 and PIK3CD levels, NRG1 induced PIPS production and cell migration could serve as biomarkers for predicting treatment response.

### Example 9

### Genetic Association of PIK3CD in Genetic Association Information Network (GAIN) Genome-Wide Association Study Dataset

To further corroborate the genetic association of PIK3CD with schizophrenia, the publicly available schizophrenia genome-wide association study dataset Genetic Association Information Network (GAIN) maintained by Foundation for the National Institutes of Health (Bethesda, Md.) was consulted.

None of the SNPs with a replicable association disclosed herein were genotyped in the GAIN sample. SNPs genotyped in our sample that showed no association to schizophrenia were consistently negative in the GAIN dataset (p>0.5).

However, rs11589267, a SNP showing association in the case-control cohort (Table 1) and which is in LD (r²=1; D′=1) with rs9430220, a SNP disclosed herein to show strong association (Table 1), showed a trend for association with schizophrenia (allelic Chi-squared; p=0.079) in the Caucasian GAIN sample. Statistical imputation of rs9430220 in GAIN based on the LD with rs11589267 provided nominal evidence of single point association with schizophrenia (allelic Chi-squared; p=0.025), however association to the risk allele was reversed.

These findings provide further support for association of genetic variation in PIK3CD to schizophrenia.

### Example 10

### IC87114, a Specific PIK3CD Inhibitor, Rescues a Cellular Phenotype Related to Schizophrenia

In-vitro migration experiments investigating the effects of a PIK3CD inhibitor (IC87114) on NRG1-induced lymphocyte (LCL) migration have been performed. The cellular phenotype is migration of lymphocytes to the chemoattractant, Neuregulin (NRG1), a key regulator of brain development. NRG1 induced LCL migration is diminished in patients with schizophrenia.

Human B-lymphoblast cells were cultured in RPMI 1640 without L-Glutamine medium (Quality Biological, Inc.) supplemented with 15% fetal bovine serum (FBS), 1% Penicillin-streptomycin, and 2% L-Glutamine in a 5% CO₂ incubator at 37° C. Prior to performing migration assay, cells were incubated for 16-18 hours in the same culture, lowering the concentration of FBS to 2%. Then the cells were washed once with HBSS followed by incubation for 60 minutes in IC87114 compound at 0.1 μM concentration using the same media without serum. Following incubations, cells were seeded into the top chamber of the HTS 5 μM Transwell®-96 Cell Migration plate (Corning) at 4×105 cell/well concentration in a 50 μl volume. NRG-1 serum free RPMI solution with antibiotics and L-Glutamine supplements (NRG-1 200 ng/ml; 160 μl) was added to the bottom chamber and incubated for 4 hours at 37° C. 75 μl of the bottom chamber fraction was collected and combined with 25 μl of cyquant/lysis buffer reagent (according to Invitrogen protocol) for 15 min at RT and in the dark, and then read with a Wallac 1420 VICTOR3 multilabel plate reader under standard fluorescein (485 nm/535 nm, 1.0 s) protocol.

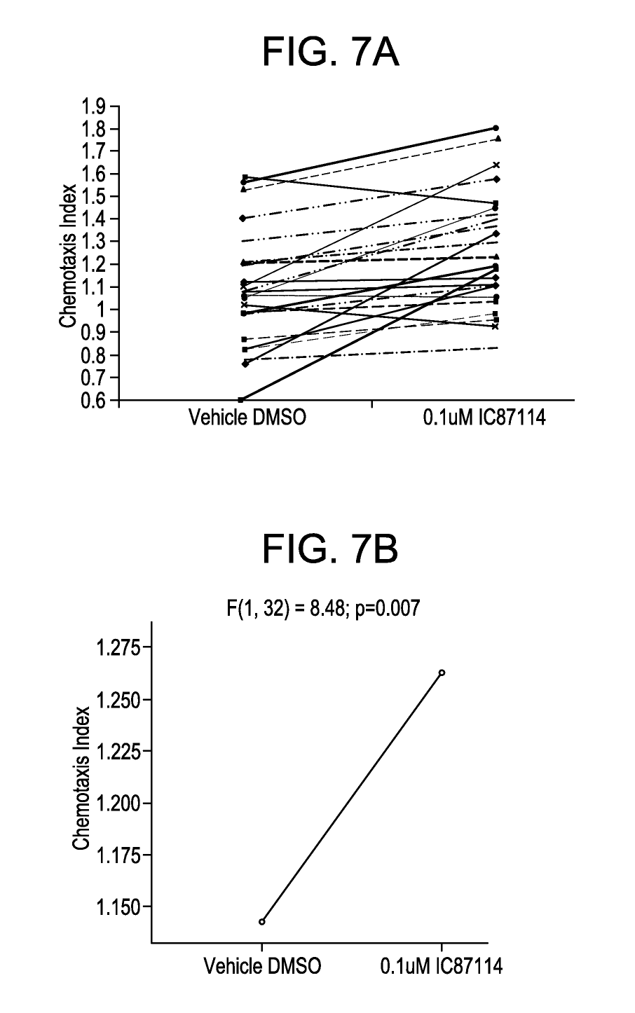

FIG. 7 shows that doses of IC87114 within the IC50 range of PIK3CD inhibition (0.1-10 uM) significantly improve LCL migration in response to NRG-1 stimulation. Data shown are 0.1 uM.

The same protocol is repeated with additional PIK3CD inhibitors, including PIK-39,

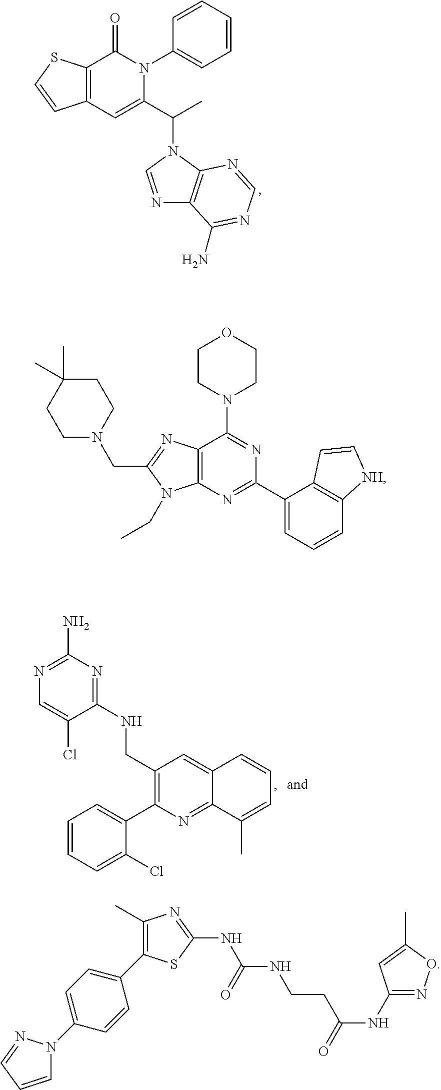

### Example 11

### Permeation of the Blood-Brain Barrier by IC87114

For compounds to be clinically useful to treat CNS disorders, they should penetrate the blood-brain barrier (BBB). Partitioning, passive blood-brain barrier permeability analysis, was conducted on IC87114 to derive a C_{brain}/C_{blood} value>2. The degree of BBB penetration is measured as the ratio of the steady-state concentrations of the drug in the brain and in the blood, expressed as log(C_{brain}/C_{blood}) or log BB. Compounds with logBB>0.3 (i.e. C_{brain}/C_{blood}>2.0) cross the BBB readily. From these studies, IC87114 represents one of these compounds.

BBB analysis was performed using Analiza assays: Based upon a method described previously, a combination of two descriptors representing the lipophilicity (as measured by octanol-buffer partitioning, logD7.4) and relative hydrophobicity (as measured by aqueous two-phase partitioning (ATPPS), N(CH₂)) of organic compounds was used to determine if that compound will permeate the blood-brain barrier.

Sample Preparation: One sample was received as 10 mM stock solutions in Dimethyl sulfoxide (DMSO) frozen on dry ice in a microtube. Upon arrival at Analiza the vial was found to be intact. The compound was stored frozen at −20° C. for approximately 24 hours. Immediately prior to analysis, the sample was thawed in a dessicator at ambient temperature, centrifuged at 3000 RPM for 5 minutes, and sonicated in a 40° C. water bath to facilitate dissolution. Following sonication, the compound appeared to be fully dissolved. The 10 mM stock solution was diluted 10-fold with DMSO, for a final nominal concentration of 1.0 mM for ATPPS analysis; 30 μL of 10 mM stock was reserved for log D analysis.

Partitioning Experiments: Partitioning in an aqueous dextran-polyethylene glycol (Dex-PEG) two-phase system containing 0.15M NaCl in 0.01M sodium phosphate buffer at pH 7.4 was performed with an Automated Signature Workstation (Analiza, Inc. Cleveland, Ohio). DMSO stock solutions, 1 mM were added to 3 wells of the DEX-PEG two-phase system per compound (30, 60, and 95 μL). The plates were sealed, vortexed on a specially designed deepwell plate mixer, and centrifuged to aid in phase settling. Relative Hydrophobicity (N(CH₂)) is then calculated from this partition coefficient. Automated Discovery Workstation, ADW (Analiza, Inc. Cleveland, Ohio) was used to remove aliquots from the two-phase systems and directly inject phases into the nitrogen detector for assay by total chemiluminescent nitrogen detection. The equimolar nitrogen response of the detector is calibrated using standards which span the dynamic range of the instrument from 0.08 to 4500 μg/ml nitrogen. Both the top and bottom phases were quantitated with respect to this calibration curve and the natural logarithm of the ratio of the concentration in the top phase to the concentration in the bottom phase is calculated as the partition coefficient from the linear regression of the compound concentration in the top phase vs. the bottom phase for the 3 dose concentrations. Relative Hydrophobicity (N(CH₂) is then calculated from this partition coefficient.

For octanol/buffer partitioning, Analiza's standard two-phase system plates were used. Octanol in equilibrium with universal buffer (composed of 0.15 M NaCl and 0.01 M each of phosphoric, boric, and acetic acids) adjusted to pH 7.4 with NaOH were used to prepare partitioning plates for the assay. This buffer provides uniform ionic composition across a wide pH range. DMSO stock solutions (10 mM, 25 μL) were added to each partitioning plate to a final concentration of 10% DMSO. The plates were sealed, vortexed on our specially designed deepwell plate mixer, and centrifuged to aid in phase settling. The assay was conducted on the ADW workstation using chemiluminescent nitrogen detection. The equimolar nitrogen response of the detector is calibrated using standards that span the dynamic range of the instrument from 0.08 to 4500 μg/ml nitrogen. Both the top and bottom phases were quantitated with respect to this calibration curve and the Logarithm of the ratio of the concentration in the top phase to the concentration in the bottom phase is calculated as the partition coefficient. In addition to reporting the directly observed Log D value, the observed Log D value was adjusted to a corrected Log D* based upon our previous work correlating Log D in the presence and absence of a fixed amount of DMSO in the partitioning system. The calculated Log D and Log D* values are corrected for any background nitrogen in the octanol buffer two-phase system and DMSO.

Calculation of Results: The probability of a compound to cross the blood-brain barrier through passive transport is calculated using the following equation.

Ln [P(CNS=“+”)/(1−P(CNS=“+”))]=−7.90+24.91*n log D*−1.10*n log D*N(CH2)

The results are presented as the ratio of the compound concentration in the brain to compound concentration in the blood (C_{brain}/C_{blood}). Values greater than 2 (>2) can be interpreted as CNS+ and values less than 0.1 (<0.1) can be interpreted as CNS−. Compounds with values between 0.1 and 2 (0.1<(C_{brain}/C_{blood})<2) may or may not passively penetrate the blood-brain barrier or may penetrate at intermediate levels.

The same protocols are repeated with additional PIK3CD inhibitors, including PIK-39,

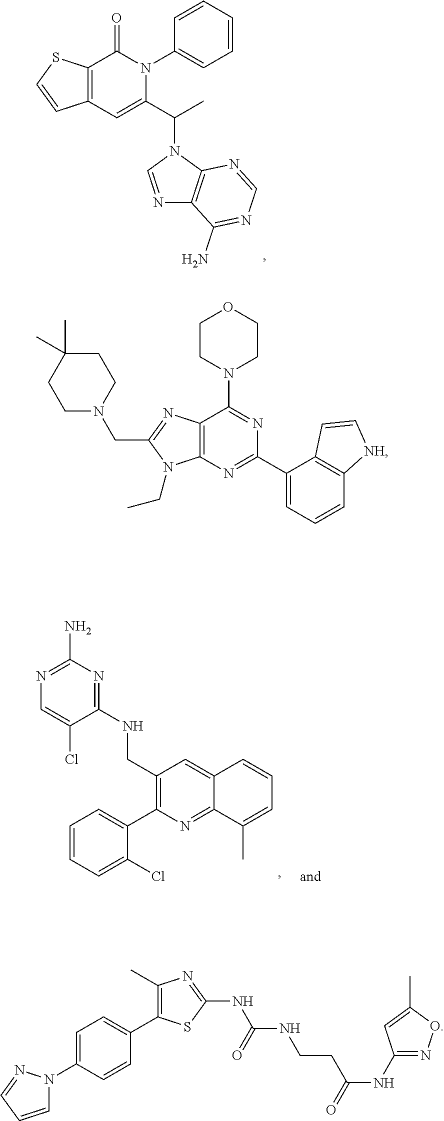

### Example 12

### Effect of PIK3CD Inhibitors on Amphetamine Induced Locomotor Abnormalities in Normal Mice and in a Genetic Mouse Model of Schizophrenia

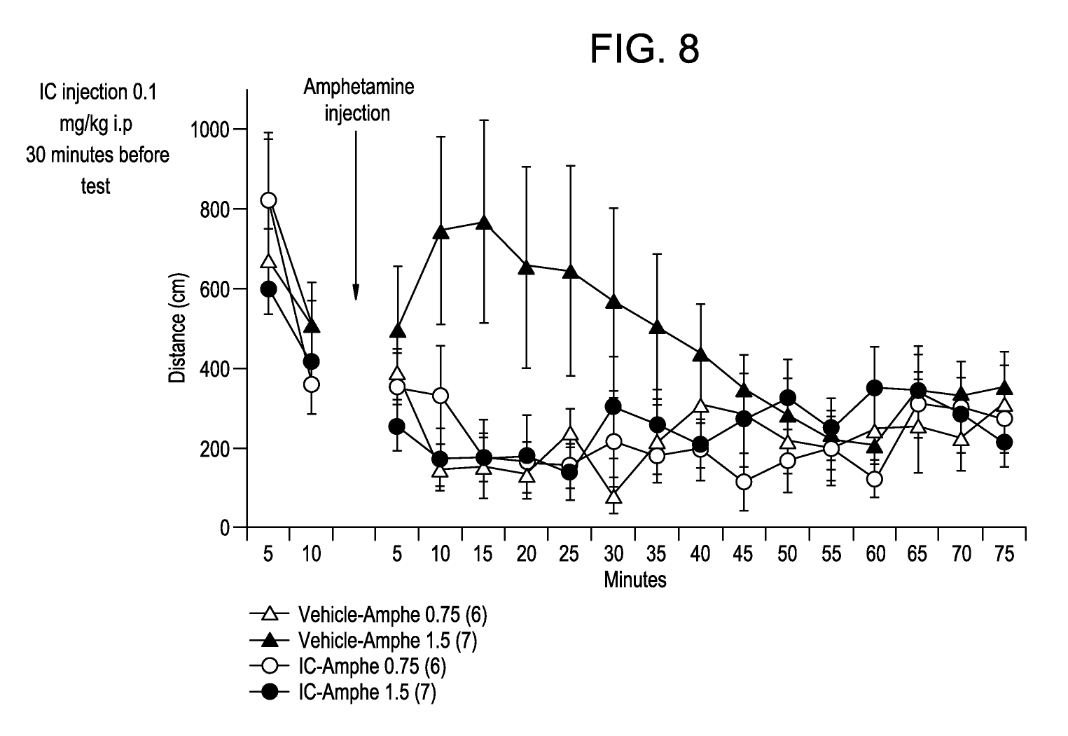

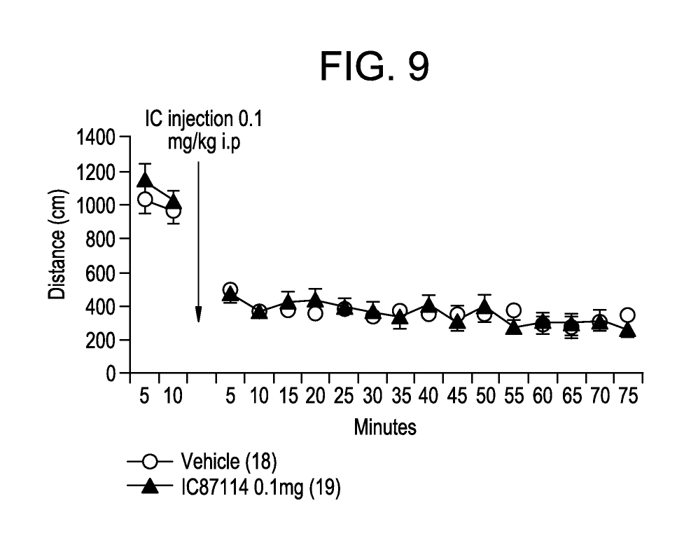

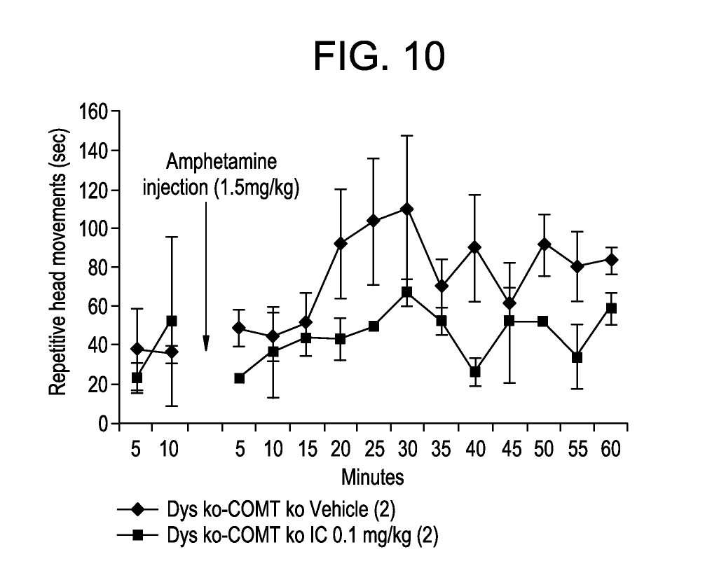

IC87114 (at doses required to specifically inhibit PIK3CD) reduces amphetamine induced locomotor abnormalities in normal mice (FIG. 8) without affecting baseline behavior (FIG. 9). Furthermore, IC87114 dramatically reduces amphetamine-induced stereotypy in a genetic mouse model of schizophrenia (FIG. 10). These data provide preclinical evidence that IC87114 effectively crosses the blood brain barrier in-vivo and ameliorates behaviors associated with schizophrenia in mice without affecting baseline behavior. These are dramatic data adding to the evidence for this therapeutic indication for this drug. Demonstration of therapeutic efficacy in a disease mouse model is a critical step in preclinical validation of antipsychotic potential.

Subjects. C57BL/6J mice were purchased from The Jackson Laboratory at 8 weeks old. All mice were group-housed (4/cage) in a climate-controlled animal facility (22±2° C.) and maintained on a 12-hr light/dark cycle, with free access to food and water. Testing was conducted in male mice, at ages 2-3 months, during the light phase of the circadian cycle. Mice were handled by the experimenter on alternate days during the week preceding the tests. At least one hour before any test manipulation, mice were habituated in a room adjacent to the testing room.

Locomotor Activity with IC87114 Treatment. Mice were tested on day 1 in an experimental apparatus consisting of four Plexiglas Digiscan automated open fields (Accuscan; 42×42×30 cm dimensions). One red light (5±2 lux) was placed overhead, evenly illuminating each open field. Each apparatus contained photobeam sensors to measure the exploratory and locomotor activity of the mice. During the first 10-minute session, mice were placed in the empty open field and allowed to explore the arena. Immediately after, mice were removed from the field and given either an injection of 0.1 mg/kg IC87114 or vehicle. They were then place back in the same open field for an additional 75 minutes. All sessions were videotaped. IC87114 was dissolved in 0.25% DMSO in saline physiological saline (vehicle) and injected i.p. in a volume of 10 ml/kg of body weight. “Vehicle-treated” mice were injected with the same volume of 0.25% DMSO in saline physiological saline.

Locomotor Activity with IC87114 and Amphetamine Treatment. On day 3, mice were tested in the same experimental apparatus and conditions as on day 1. Thirty minutes before the start of the first 10-minute session the mice were injected with either IC87114 (0.1 mg/kg) or vehicle, the same as the treatment received on day 1. During the first 10-minute session mice were placed in the same empty open field as day 1. Immediately after, mice were removed from the field and given an injection of D-amphetamine sulphate (either 0.75 mg/kg or 1.5 mg/kg, i.p., Sigma-Aldrich, St. Louis, Mo., USA). They were then placed back in the same open field and allowed to explore for an additional 75 minutes. Amphetamine was dissolved in physiological saline and injected in a volume of 10 ml/kg. IC87114 in COMT*Dysbindin double knockout mice.

Subjects. All procedures were approved by the NIMH Animal Care and Use Committee and followed the NIH Guidelines “Using Animals in Intramural Research.” To derive the double knockout mice, the lines of COMT knockout mice and dysbindin knockout mice previously described were used. COMT*dysbindin double knockout mice (COMT−/− dys−/−) were littermates and bred by double heterozygote mating (dCOMT+/−dys+/− with cCOMT+/−dys+/−). Mice were identified by PCR analysis of tail DNA. Mice were group-housed (2-4/cage) in a climate-controlled animal facility (22±2° C.) and maintained on a 12-hr light/dark cycle, with free access to food and water. Testing was conducted in male mice, 7 months old, during the light phase. Experimenters were blind to the genotype during behavioral testing. Mice were handled by the experimenter on alternate days during the week preceding the tests. At least one hour before any test manipulation, mice were habituated in a room adjacent to the testing room.

Stereotypy behavior with IC87114 and Amphetamine Treatment. Mice were tested on days 1 and 3 in an experimental apparatus consisting of four Plexiglas Digiscan automated open fields (Accuscan; 42×42×30 cm dimensions). One red light (5±2 lux) was placed overhead, evenly illuminating each open field. Each apparatus contained photobeam sensors to measure the exploratory and locomotor activity of the mice. Thirty minutes before the start of the first 10-minute session each mouse was assigned to receive a single injection of either vehicle or IC87114 (0.1 mg/kg) according to a full Latin-square design, wherein each mouse was randomly treated with vehicle or IC87114. IC87114 was dissolved in 0.25% DMSO in saline physiological saline (vehicle) and injected intraperitoneally (i.p.) in a volume of 10 ml/kg of body weight. “Vehicle-treated” mice were injected with the same volume of 0.25% DMSO in saline physiological saline. During the first 10-minute session, mice were placed in the empty open field and allowed to explore the arena. Immediately after, mice were removed from the field and given an injection of D-amphetamine sulphate (1.5 mg/kg, i.p., Sigma-Aldrich, St. Louis, Mo., USA). They were then placed back in the same open field and allowed to explore for an additional 75 minutes. Amphetamine was dissolved in physiological saline and injected in a volume of 10 ml/kg. All sessions were videotaped. Stereotypy was scored from videotapes by an observer blind to the treatments and genotype conditions of each mouse. Stereotypy behaviors were defined as time spent in focused engagement in repetitive head movements (including sniffing, bobbing, weaving, swaying, stretching back-and-forth or left-and-right), while in a stationary posture.

Statistical analysis. Results were expressed as mean±standard error of the mean (S.E.M.) throughout. Student T-test, Two- or Three-Way analysis of variance (ANOVA) was used. Post-hoc analyses for individual group comparisons employed Newman-Keuls analyses.

Detailed Results. IC87114 was tested in wild-type C57BL/6J mice in an open field arena.

Baseline activity: Analysis of the distance traveled during the first 10 minutes prior to experimental condition, showed no difference in locomotor activity in the two groups of mice assigned to receive either vehicle or IC87114 (0.1 mg/kg; FIG. 3B). Analysis of the distance traveled during the 75 minutes following vehicle or IC87114 (0.1 mg/kg) also did not show any drug-treatment effect.

Amphetamine administration: Analysis of the distance traveled during the 75 minutes following amphetamine injections revealed a significant interaction of pretreatment (vehicle or IC87114), amphetamine dose (0.75 mg/kg or 1.5 mg/kg) and session time (3-Way ANOVA; F_{14,308}=3.04; P<0005), whereby, IC87114 pretreatment blocked the amphetamine-induced increase in locomotor activity (P<0.05; 8).

IC87114 in COMT*Dysbindin double knockout mice. Amphetamine injection in the COMT*dysbindin double null mutant mice produced stereotypy behaviors which were diminished by IC87114 pretreatment

The same protocols are repeated with additional PIK3CD inhibitors, including PIK-39,

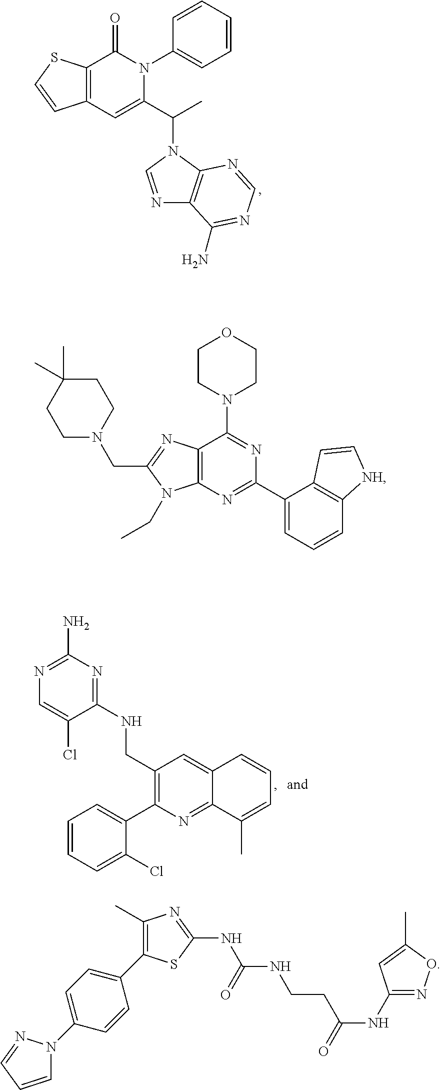

Representative selective PIK3CD inhibitors, for use in the compositions and methods described herein, satisfy one or more of the following Formulas I-X, or are a pharmaceutically acceptable salt and/or hydrate of such a compound. Variables within each Formula are defined herein independently of variables in the other Formulas (i.e., the variable R₁, for example, may carry a different definition in different Formulas).

It may be helpful in the understanding of the present disclosure to set forth definitions of certain terms used herein.

The terms “a” and “an” do not denote a limitation of quantity, but rather denote the presence of at least one of the referenced items. The terms “comprising”, “having”, “including”, and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and the all ranges, including endpoints, are independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Compounds are described using standard nomenclature. All compounds are understood to include all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C. Compounds described herein may contain one or more asymmetric elements such as stereogenic centers, stereogenic axes and the like (e.g., asymmetric carbon atoms), so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers (i.e., optically active forms) can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

The term “substituted” means that any one or more hydrogens on the designated atom or group is replaced with a selection from the indicated group (a “substituent”), provided that the designated atom's normal valence is not exceeded. When the substituent is oxo e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

The term “alkyl” includes both branched and straight chain saturated aliphatic hydrocarbon groups, having the specified number of carbon atoms. The term C₁-C₆alkyl (also written as C₁₋₆alkyl) means an alkyl group having from 1 to about 6 carbon atoms, e.g., methyl, ethyl, n-propyl, isopropyl, t-butyl, etc. “Alkylene” refers to a divalent alkyl linking moiety; for example, C₁alkylene refers to —CH₂— and a C₂alkylene is —CH₂CH₂— or —CH(CH₃)—. An alkylene moiety may be indicated along with the moiety to which it is linked (e.g., C₁₋₃alkylenearyl, which is an aryl moiety linked via a C₁₋₃alkylene group). Such a group may also be indicated as “alkyl” following another group, as in arylC₁₋₃alkyl, which refers to the same substituent as C₁₋₃alkylenearyl. “Alkenyl” refers to straight or branched chain alkene groups, which comprise at least one unsaturated carbon-carbon double bond. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively. The term “alkoxy” means an alkyl group as described above attached via an oxygen bridge. Alkoxy groups include C₁-C₆alkoxy and C₁-C₄alkoxy groups, which have from 1 to 6 or from 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups.

“Halo” or “halogen” means fluoro, chloro, bromo, or iodo. The term “oxo” means a keto group (C═O). An oxo group that is a substituent of a nonaromatic carbon atom results in a conversion of CH₂, to C(═O). Similarly, a “thioxo” group is a C═S group. “Acyl” means any group of the form RC(═O)— where R is an organic group. Acetyl is a representative acyl group. “Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl. “Perfluoroalkyl” refers to an alkyl group in which each hydrogen is replaced by fluorine.

“Carbocycle” refers to a group that comprises at least one ring, wherein all ring members of all rings are carbon. Carbocycles include aryl and cycloalkyl moieties. “Aryl” refers to a cyclic moiety in which all ring members are carbon and at least one ring is aromatic. Aryl groups include monocycles (i.e., phenyl) as well as bicyclic groups (e.g., naphthyl or biphenylyl) and moieties with additional rings. “Cycloalkyl” refers to a cyclic group comprising one or more rings in which no ring is aromatic and all ring members are carbon. C₃-C₈cycloalkyl groups, for example, comprise a single ring with from 3 to 8 ring members, or a bridged ring with from 3 to 8 ring members, or a bicyclic group in which the total number of ring members ranges from 3 to 8. A “heterocycle” or “heterocyclic ring” is a saturated, partially saturated, or aromatic ring that comprises at least one (typically 1, 2 or 3) heteroatom ring members independently chosen from N, O and S, with remaining ring members being carbon. 5-membered heterocycles contain 5 ring members, at least one of which is a heteroatom. A “heteropolycyclic ring system” is a heterocyclic moiety that comprises more than one ring, at least one ring of which is a heterocycle. A “heterocycloalkyl” is a saturated cyclic group containing from 1 to about 3 heteroatoms chosen from N, O, and S, with remaining ring atoms being carbon. Examples of 5-membered heterocycloalkyl groups include tetrahydrofuranyl and pyrrolidinyl groups. A “heteroaryl” group is an aromatic cyclic group containing at least one heteroatom (e.g., from 1 to about 3 heteroatoms) chosen from N, O, and S, with remaining ring atoms being carbon. Heteroaryl groups may comprise more than one ring; in such cases, one or more of the rings may be heterocycles. Examples of heteroaryl groups include pyrimidinyl, pyridinyl, indolyl, and quinazolinyl groups.

Unless otherwise indicated, the term “compound of Formula X,” where “X” may be any formula number, is intended to refer to compounds that satisfy the recited formula, as well as pharmaceutically acceptable salts and/or hydrates of such compounds. A “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutically acceptable anions for use in salt formation include, but are not limited to, acetate, 2-acetoxybenzoate, ascorbate, benzoate, bicarbonate, bromide, calcium edetate, carbonate, chloride, citrate, dihydrochloride, diphosphate, ditartrate, edetate, estolate (ethylsuccinate), formate, fumarate, gluceptate, gluconate, glutamate, glycolate, glycollylarsanilate, hexylresorcinate, hydrabamine, hydrobromide, hydrochloride, hydroiodide, hydroxymaleate, hydroxynaphthoate, iodide, isethionate, lactate, lactobionate, malate, maleate, mandelate, methylbromide, methylnitrate, methylsulfate, mucate, napsylate, nitrate, pamoate, pantothenate, phenylacetate, phosphate, polygalacturonate, propionate, salicylate, stearate, subacetate, succinate, sulfamate, sulfanilate, sulfate, sulfonates including besylate (benzenesulfonate), camsylate (camphorsulfonate), edisylate (ethane-1,2-disulfonate), esylate (ethanesulfonate), 2-hydroxyethylsulfonate, mesylate (methanesulfonate), triflate (trifluoromethanesulfonate) and tosylate (p-toluenesulfonate), tannate, tartrate, teoclate and triethiodide. Similarly, pharmaceutically acceptable cations for use in salt formation include, but are not limited to ammonium, benzathine, chloroprocaine, choline, diethanolamine, ethylenediamine, meglumine, procaine, and metals such as aluminum, calcium, lithium, magnesium, potassium, sodium, and zinc. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, methanol, isopropanol or acetonitrile, is preferred.

“Pharmaceutical compositions” means compositions comprising at least one active agent, such as a compound or salt of the invention, and at least one other substance, such as a carrier. Pharmaceutical compositions meet the U.S. FDA's GMP (good manufacturing practice) standards for human or non-human drugs. “Carrier”, in the context of a compound, means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

An allele “carrier” means an individual that is a heterozygote at a polymorphic site.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient. The methods of the invention embrace various modes of treating an animal subject, preferably a mammal, more preferably a primate, and still more preferably a human. Among the mammalian animals that can be treated are, for example, companion animals (pets), including dogs and cats; farm animals, including cattle, horses, sheep, pigs, and goats; laboratory animals, including rats, mice, rabbits, guinea pigs, and nonhuman primates; and zoo specimens. Nonmammalian animals include, for example, birds, fish, reptiles, and amphibians.

“Providing” means giving, administering, selling, distributing, transferring (for profit or not), manufacturing, compounding, or dispensing.

“Treating” means preventing a disorder from occurring in an animal that can be predisposed to the disorder, but has not yet been diagnosed as having it; inhibiting the disorder, i.e., arresting its development; relieving the disorder, i.e., causing its regression; or ameliorating the disorder, i.e., reducing the severity of symptoms associated with the disorder.

“Disorder” encompasses medical disorders, diseases, conditions, syndromes, and the like, without limitation. A “therapeutically effective amount” of a pharmaceutical composition means an amount effective, when administered to a patient, to provide a therapeutic benefit such as an amelioration of symptoms, e.g., an amount effective to decrease the symptoms of a CNS disorder.

A “significant change” is any detectable change that is statistically significant in a standard parametric test of statistical significance such as Student's T-test, where p<0.05.

While the invention has been described with reference to a preferred embodiment, it will be understood by those skilled in the art that various changes may be made and equivalents may be substituted for elements thereof without departing from the scope of the invention. Also, many modifications may be made to adapt a particular situation or material to the teachings herein without departing from essential scope thereof. Therefore, it is intended that the claims not be limited to the particular embodiment disclosed as the best mode contemplated for carrying out this invention, but that the invention will include all embodiments falling within the scope of the appended claims.

1. A method for treating a patient in need of treatment for a CNS disorder, comprising administering to the patient a therapeutically effective amount of a selective PIK3CD inhibitor, and thereby reducing a symptom of the CNS disorder in the patient.

2. The method of claim 1, wherein the CNS disorder is schizophrenia, psychosis, or a cognitive disorder.

3. The method of claim 2, wherein the disorder is schizophrenia and the symptom is delusions, hallucinations, disorganized speech, catatonic behavior, a cognitive symptom, or a combination thereof.

4. The method of claim 2, wherein the disorder is psychosis and the symptom is delusions, hallucinations, or a combination thereof.

5. The method of claim 1, wherein the selective PIK3CD inhibitor is a compound of Formula I: 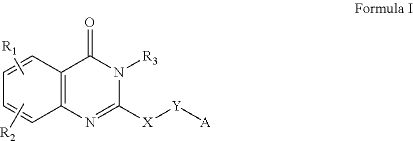
or a pharmaceutically acceptable salt thereof, wherein:
A is an optionally substituted monocyclic 5-membered heterocyclic ring system containing two or three nitrogen atoms or a bicyclic ring system containing two nitrogen atoms and one ring of the bicyclic system is aromatic, or a ring of the formula 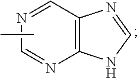
X is C(R_{b})₂, CH₂CHR_{b}, or CH═C(R_{b});
Y is NH, absent, S, SO, or SO₂;
R₁ and R₂, independently, are selected from hydrogen, C₁₋₆alkyl, aryl, heteroaryl, halo, NHC(═O)C₁₋₃alkyleneN(Rₐ)₂, NO₂, ORₐ, CF₃, OCF₃, N(Rₐ)₂, CN, OC(═O)Rₐ, C(═O)Rₐ, C(═O)ORₐ, arylOR_{b}, Het, NRₐC(═O)C₁₋₃alkyleneC(═O)ORₐ, C(═O)ORₐ, C₁₋₃alkyleneN(Rₐ)₂, arylOC(═O)Rₐ, C₁₋₄alkyleneC(═O)ORₐ, OC₁₋₄alkyleneC(═O)ORₐ, C₁₋₄alkyleneOC₁₋₄ alkyleneC(═O)ORₐ, C(═O)NRₐSO₂Rₐ, C₁₋₄alkyleneN(Rₐ)₂, C₂₋₆alkenylene-N(Rₐ)₂, C(═O)NRₐC₁₋₄-alkyleneORₐ, C(═O)NRₐC₁₋₄alkylene-Het, OC₂₋₄alkyleneN(Rₐ)₂, OC₁₋₄alkyleneCH(OR_{b})CH₂N(Rₐ)₂, OC₁₋₄alkyleneHet, OC₂₋₄alkyleneORₐ, OC₂₋₄alkylene-NRₐC(═O)ORₐ, NRₐ C₁₋₄alkyleneN(Rₐ)₂, NRₐC(═O)Rₐ, NRₐC(═O)N(Rₐ)₂, N(SO₂C₁₋₄alkyl)₂, NRₐC(SO₂C₁₋₄ alkyl), SO₂N(Rₐ)₂, OSO₂CF₃, C₁₋₃alkylenearyl, C₁₋₄alkyleneHet, C₁₋₆alkyleneOR_{b}, C₁₋₃alkyleneN(Rₐ)₂, C(═O)N(Rₐ)₂, NHC(═O)C₁-C₃alkylenearyl, C₃₋₈cycloalkyl, C₃₋₈heterocycloalkyl, arylOC₁₋₃alkyleneN(Rₐ)₂, arylOC(═O)R_{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR_{b}, C(═O)C₁₋₄alkyleneHet, and NHC(═O)haloC₁₋₆alkyl;
R₃ is optionally substituted aryl;
each Rₐ is selected from hydrogen, C₁₋₆alkyl, C₃₋₈cycloalkyl, C₃₋₈heterocycloalkyl, C₁₋₃ alkyleneN(R_{c})₂, aryl, arylC₁₋₃alkyl, C₁₋₃alkylenearyl, heteroaryl, heteroarylC₁₋₃alkyl, and C₁₋₃alkyleneheteroaryl;
or two Rₐ groups are taken together to form a 5- or 6-membered ring, optionally containing at least one heteroatom;
each R_{b} is selected from hydrogen, C₁₋₆alkyl; R_{c} is selected from hydrogen, C₁₋₆alkyl, C₃₋₈cycloalkyl, aryl, and heteroaryl; and
each Het is selected from 1,3-dioxolane, 2-pyrazoline, pyrazolidine, pyrrolidine, piperazine, pyrroline, 2H-pyran, 4H-pyran, morpholine, thiomorpholine, piperidine, 1,4-dithiane, and 1,4-dioxane, and optionally substituted with C₁₋₄alkyl or C(═O)ORₐ.

6. The method of claim 5, wherein the selective PIK3CD inhibitor is 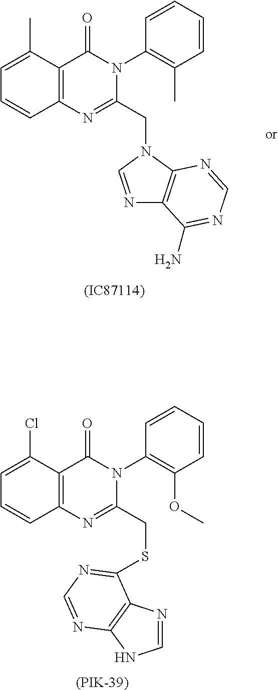
or a pharmaceutically acceptable salt thereof.

7. The method of claim 1, wherein the selective PIK3CD inhibitor is a compound of Formula II: 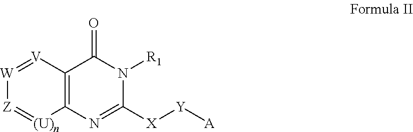
or a pharmaceutically acceptable salt thereof, wherein:
U, V, W, and Z, independently, are selected from CRₐ, N, NR_{b}, and O; or at least one of U, V, W and Z is N, and the others of U, V, W and Z are selected from the group consisting of CRₐ, NR_{b}, S, and O; and at least one, but not all, of U, V, W, and Z is different from CRₐ;
A is an optionally substituted monocyclic or bicyclic ring system containing at least two nitrogen atoms as ring members, and at least one ring of the system is aromatic;
X is C(R_{c})₂, C(R_{c})₂C(R_{c})₂, CH₂CHR_{c}, CHR_{c}CHR_{c}, CHR_{C}CH₂, CH═C(R_{c}), C(R_{c})═C(R_{c}), or C(R_{c})═CH;
Y is absent, S, SO, SO₂, NH, N(R_{c}), O, C(═O), OC(═O), C(═O)O, or NHC(═O)CH₂S;
R₁ is selected from H, substituted or unsubstituted C₁₋₁₀alkyl, substituted or unsubstituted C₂₋₁₀alkenyl, substituted or unsubstituted C₂₋₁₀alkynyl, substituted or unsubstituted C₁₋₆perfluoroalkyl, substituted or unsubstituted C₃₋₈cycloalkyl, substituted or unsubstituted C₃₋₈heterocycloalkyl, substituted or unsubstituted C₁₋₄alkyleneC₃₋₈cycloalkyl, substituted or unsubstituted aryl, substituted or unsubstituted heteroaryl, substituted or unsubstituted arylC₁₋₄alkyleneOR_{c}, substituted or unsubstituted heteroarylC₁₋₄alkyleneN(R_{d})₂, substituted or unsubstituted heteroarylC₁₋₄alkyleneORₑ, substituted or unsubstituted C₁₋₃alkyleneheteroaryl, substituted or unsubstituted C₁₋₃alkylenearyl, substituted or unsubstituted arylC₁₋₆alkyl, arylC₁₋₄alkyleneN(R_{d})₂, C₁₋₄alkyleneC(═O)C₁₋₄alkylenearyl, C₁₋₄alkyleneC(═O)C₁₋₄alkyleneheteroaryl, C₁₋₄alkyleneC(═O)heteroaryl, C₁₋₄alkyleneC(═O)N(R_{d})₂, C₁₋₆alkyleneOR_{d}, C₁₋₄alkyleneNRₐC(═O)R_{d}, C₁₋₄alkyleneOC₁₋₄alkyleneOR_{d}, C₁₋₄alkyleneN(R_{d})₂, C₁₋₄alkyleneC(═O)OR_{d}, and C₁₋₄alkyleneOC₁₋₄alkyleneC(═O)OR_{d};
each Rₐ is independently selected from H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, C₁₋₃alkylenearyl, substituted or unsubstituted heteroaryl, substituted or unsubstituted heteroarylC₁₋₃alkyl, substituted or unsubstituted C₁₋₃alkyleneheteroaryl, halo, NHC(═O)C₁₋₃alkyleneN(R_{d})₂, NO₂, ORₑ, CF₃, OCF₃, N(R_{d})₂, CN, OC(═O)R_{d}, C(═O)R_{d}, C(═O)OR_{d}, arylORₑ, NR_{d}C(═O)C₁₋₃alkyleneC(═O)OR_{d}, arylOC₁₋₃alkyleneN(R_{d})₂, arylOC(═O)R_{d}, C₁₋₄alkyleneC(═O)OR_{d}, OC₁₋₄alkyleneC(═O)OR_{d}, C₁₋₄alkyleneOC₁₋₄alkyleneC(═O)OR_{d}, C(═O)NR_{d}SO₂R_{d}, C₁₋₄alkyleneN(R_{d})₂, C₂₋₆alkenyleneN(R_{d})₂, C(═O)NR_{d}C₁₋₄alkyleneORₑ, C(═O)NR_{d}C₁₋₄alkyleneheteroaryl, OC₁₋₄alkyleneN(R_{d})₂, OC₁₋₄alkyleneCH(ORₑ)CH₂N(R_{d})₂, OC₁₋₄alkyleneheteroaryl, OC₂₋₄alkyleneORₑ, OC₂₋₄alkyleneNR_{d}C(═O)OR_{d}, NRₐC₁₋₄alkyleneN(R_{d})₂, NRₐC═O)R_{d}, NRₐC(═O)N(R_{d})₂, N(SO₂C₁₋₄alkyl)₂, NRₐ(SO₂C₁₋₄alkyl), SO₂N(R_{d})₂, OSO₂CF₃, C₁₋₃alkylenearyl, C₁₋₄ alkyleneheteroaryl, C₁₋₆alkyleneORₑ, C(═O)N(R_{d})₂, NHC(═O)C₁₋₃alkylenearyl, arylOC₁₋₃alkyleneN(R_{c})₂, arylOC(═O)R_{d}, NHC(═O)C₁₋₃alkyleneC₃₋₈heterocycloalkyl, NHC(═O)C₁₋₃alkyleneheteroaryl, OC₁₋₄alkleneOC₁₋₄alkyleneC(═O)OR_{d}, C(═O)C₁₋₄alkyleneheteroaryl, and NHC(═O)haloC₁₋₆alkyl;
each R_{b} is independently absent or selected from H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, substituted or unsubstituted arylC₁₋₃alkyl, C₁₋₃alkylenearyl, substituted or unsubstituted heteroaryl, heteroarylC₁₋₃alkyl, substituted or unsubstituted C₁₋₃alkyleneheteroaryl, C(═O)R_{d}, C(═O)OR_{d}, arylORₑ, arylOC₁₋₃alkyleneN(R_{d})₂, arylOC(═O)R_{d}, C₁₋₄alkyleneC(═O)OR_{d}, C₁₋₄alkyleneOC₁₋₄alkyleneC(═O)OR_{d}, C(═O)NR_{d}SO₂R_{d}, C₁₋₄alkyleneN(R_{d})₂, C₂₋₆alkenyleneN(R_{d})₂, C(═O)NR_{d}C₁₋₄ alkyleneORₑ, C(═O)NR_{d}C₁₋₄alkyleneheteroaryl, SO₂N(R_{d})₂, C₁₋₃alkylenearyl, C₁₋₄ alkyleneheteroaryl, C₁₋₆alkyleneORₑ, C₁₋₃alkyleneN(R_{d})₂, C(═O)N(R_{d})₂, arylOC₁₋₃alkyleneN(R_{d})₂, arylOC(═O)R_{d}, and C(═O)C₁₋₄alkyleneheteroaryl;
each R_{c} is independently selected from H, substituted or unsubstituted C₁₋₁₀alkyl, substituted or unsubstituted C₃₋₈cycloalkyl, substituted or unsubstituted C₃₋₈heterocycloalkyl, substituted or unsubstituted C₁₋₄alkyleneN(R_{d})₂, substituted or unsubstituted C₁₋₃alkyleneheteroC₁₋₃alkyl, substituted or unsubstituted arylheteroC₁₋₃alkyl, substituted or unsubstituted aryl, substituted or unsubstituted heteroaryl, substituted or unsubstituted arylC₁₋₃alkyl, substituted or unsubstituted heteroarylC₁₋₃alkyl, C₁₋₃alkylenearyl, substituted or unsubstituted C₁₋₃ alkyleneheteroaryl, C(═O)R_{d}, and C(═O)OR_{d};
or two R_{c} on the same atom or on adjacent connected atoms can cyclize to form a ring having 3-8 ring members, which ring is optionally substituted and may include up to two heteroatoms selected from NR_{d}, O, and S as ring members;
each R_{d} is independently selected from H, substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈heterocycloalkyl, substituted or unsubstituted C₁₋₃alkyleneN(Rₑ)₂, aryl, substituted or unsubstituted arylC₁₋₃alkyl, substituted or unsubstituted C₁₋₃alkylenearyl, substituted or unsubstituted heteroaryl, substituted or unsubstituted heteroarylC₁₋₃alkyl, and substituted or unsubstituted C₁₋₃alkyleneheteroaryl;
or two R_{d} groups are taken together with the nitrogen to which they are attached to form a 5- or 6-membered ring, optionally containing a second heteroatom that is N, O, or S;
each Rₑ is selected from H, substituted or unsubstituted C₁₋₆alkyl, substituted or unsubstituted C₃₋₈cycloalkyl, substituted or unsubstituted aryl, and substituted or unsubstituted hetero aryl,
or two Rₑ groups are taken together with the nitrogen to which they are attached to form a 5- or 6-membered ring, optionally containing a second heteroatom that is N, O, or S;
the A, R₁, Rₐ, R_{b}, R_{c}, and R_{d}, independently, are optionally substituted with one to three substituents selected from C₁₋₁₀alkyl, C₂₋₁₀alkenyl, C₂₋₁₀alkynyl, C₃₋₈cycloalkyl, C₃₋₈heterocycloalkyl, C₁₋₆alkyleneORₑ, C₁₋₄alkyleneN(Rₑ)₂, aryl, C₁₋₃alkylenearyl, heteroaryl, C(═O)ORₑ, C(═O)Rₑ, OC(═O)Rₑ, halo, CN, CF₃, NO₂, N(Rₑ)₂, ORₑ, OC₁₋₆ perfluoralkyl, OC(═O)N(Rₑ)₂, C(═O)N(Rₑ)₂, SRₑ, SO₂Rₑ, SO₃Rₑ, oxo(═O), and CHO; and
n is 0 or 1.

8. The method of claim 7, wherein the selective PIK3CD inhibitor is 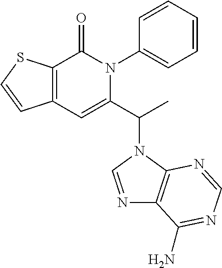
or a pharmaceutically acceptable salt thereof.

9. The method of claim 1, wherein the selective PIK3CD inhibitor is a compound of Formula III or Formula IV: 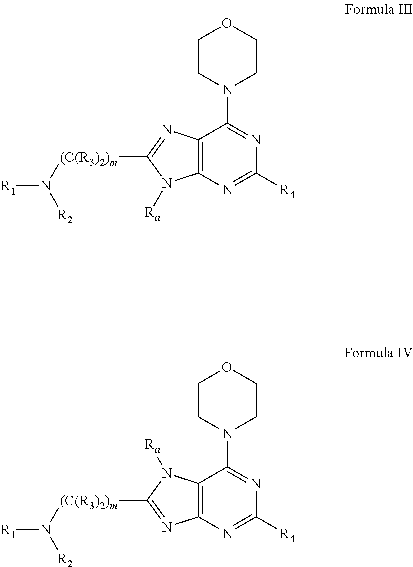
or a pharmaceutically acceptable salt thereof, wherein:
R₁ and R₂ form, together with the N atom to which they are attached:
(a) a 4- to 7-membered saturated N-containing heterocyclic ring which includes 0 or 1 additional heteroatoms selected from N, S, and O, the ring being unsubstituted or substituted;
(b) a 4- to 7-membered saturated N-containing heterocyclic ring which includes 0 or 1 additional heteroatoms selected from N, S and O, the ring being fused to a second ring selected from a 4- to 7-membered saturated N-containing heterocyclic ring as defined above, a 5- to 12-membered unsaturated heterocyclic ring, a 5- to 7-membered saturated O-containing heterocyclic ring, a 3- to 12-membered saturated carbocyclic ring and an unsaturated 5- to 12-membered carbocyclic ring to form a heteropolycyclic ring system, the heteropolycyclic ring system being unsubstituted or substituted;
(c) a 4- to 7-membered saturated N-containing heterocyclic ring which includes 0 or 1 additional heteroatoms selected from N, S and O and which further comprises, linking two constituent atoms of the ring, a bridgehead group selected from —(CR′₂)ₙ— and —(CR′₂), —O—(CR′₂)ₛ— wherein each R′ is independently H or C₁-C₆alkyl, n is 1, 2 or 3, r is 0 or 1, and s is 0 or 1, the remaining ring positions being unsubstituted or substituted; or
(d) a group of the formula: 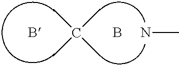
wherein ring B is a 4- to 7-membered saturated N-containing heterocyclic ring which includes 0 or 1, additional heteroatoms selected from N, S and O and ring B′ is a 3- to 12-membered saturated carbocyclic ring, a 5- to 7-membered saturated O-containing heterocyclic ring or a 4- to 7-membered saturated N-containing heterocyclic ring as defined above, each of B and B′ being unsubstituted or substituted;
or one of R₁ and R₂ is C₁-C₆alkyl and the other of R₁ and R₂ is selected from a 3- to 12-membered saturated carbocyclic group which is unsubstituted or substituted, a 5- to 12-membered unsaturated carbocyclic group which is unsubstituted or substituted, a 5- to 12-membered unsaturated heterocyclic group which is unsubstituted or substituted, a 4- to 12-membered saturated heterocyclic group which is unsubstituted or substituted and a C₁-C₆alkyl group which is substituted by a group selected from a 3- to 12-membered saturated carbocyclic group which is unsubstituted or substituted, a 5- to 12-membered unsaturated carbocyclic group which is unsubstituted or substituted, a 5- to 12-membered unsaturated heterocyclic group which is unsubstituted or substituted and a 4- to 12-membered saturated heterocyclic group which is unsubstituted or substituted;
m is 0, 1, or 2;
R₃ is H or C₁-C₆ alkyl;
Rₐ is selected from R, C(O)OR, C(O)NR₂, halo(C₁-C₆)alkyl, and SO₂R, SO₂NR₂, wherein each R is independently H or C₁-C₆ alkyl which is unsubstituted or substituted; and
R₄ is an indole group which is unsubstituted or substituted.

10. The method of claim 9, wherein the selective PIK3CD inhibitor is: 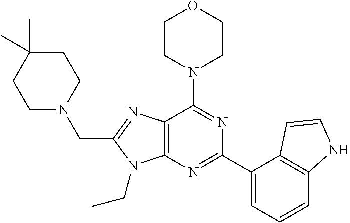
or a pharmaceutically acceptable salt thereof.

11. The method of claim 1, wherein the selective PIK3CD inhibitor is a compound of Formula V: 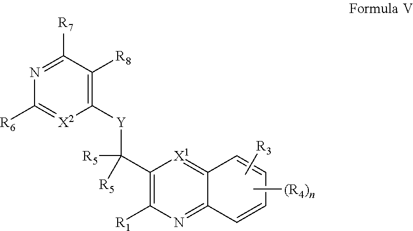
or a pharmaceutically acceptable salt thereof, wherein:
X¹ is C(R₉) or N;
X² is C(R₁₀) or N;
Y is N(R₁₁), O or S;
n is 0, 1, 2, or 3;
R₁ is a direct-bonded or oxygen-linked saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0 or 1, R₂ substituents, and the ring is additionally substituted by 0, 1, 2 or 3 substituents independently selected from halo, nitro, cyano, C₄alkyl, OC₁₋₄alkyl, OC₁₋₄haloalkyl, NHC₁₋₄alkyl, N(C₁₋₄-alkyl)C₁₋₄alkyl and C₁₋₄haloalkyl;
R₂ is selected from halo, C₁₋₄haloalkyl, cyano, nitro, —C(═O)Rₐ, C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, NRₐRₐ, N(Rₐ)C(═O)Rₐ, —N(Rₐ)C(═O)ORₐ, —N(Rₐ)C(═O)NRₐRₐ, N(Rₐ)C(═NRONRₐRₐ, —N(Rₐ)S(═O)₂Rₐ, —N(Rₐ)S(═O)₂NRₐRₐ, —NRₐC₂₋₆alkylNRₐRₐ and —NRₐC₂₋₆alkylORₐ;
or R₂ is selected from C₁₋₆alkyl, phenyl, benzyl, heteroaryl, heterocycle, —(C₁₋₃alkyl)heteroaryl, —(C₁₋₃alkyl)heterocycle, —O(C₁₋₃alkyl)heteroaryl, —O(C₁₋₃alkyl)heterocycle, —NRₐ(C₁₋₃alkyl)heteroaryl, —NRₐ(C₁₋₃alkyl)heterocycle, —(C₁₋₃alkyl)phenyl, —O(C₁₋₃alkyl)phenyl and —NRₐ(C₁₋₃alkyl)phenyl all of which are substituted by 0, 1, 2 or 3 substituents independently selected from C₁₋₄haloalkyl, OC₁₋₄alkyl, Br, Cl, F, I and C₁₋₄alkyl;
R₃ is selected from H, halo, C₁₋₄haloalkyl, cyano, nitro, —C(═O)Rₐ, —C(═O)ORₐ, C(═O)NRₐRₐ, —C(═NRₐ)NRₐRₐ, —ORₐ, —OC(═O)Rₐ, —OC(═O)NRₐRₐ, —OC(═O)N(ROS(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, NRₐRₐ, N(Rₐ)C(═O)Rₐ, —N(Rₐ)C(═O)ORₐ, —N(Rₐ)C(═O)NRₐRₐ, N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ, —NRₐC₂₋₆alkylORₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I, and C₁₋₆alkyl;
R₄ is, independently, in each instance, halo, nitro, cyano, C₁₋₄alkyl, OC₁₋₄alkyl, OC₁₋₄haloalkyl, NHC₁₋₄alkyl, N(C₁₋₄alkyl)C₁₋₄alkyl, or C₁₋₄haloalkyl;
R₅ is, independently, in each instance, H, halo, C₁₋₆alkyl, C₁₋₄haloalkyl, or C₁₋₆alkyl substituted by 1, 2 or 3 substituents selected from halo, cyano, OH, OC₁₋₄alkyl, C₁₋₄alkyl, C₁₋₃haloalkyl, OC₁₋₄alkyl, NH₂, NHC₄alkyl, N(C₁₋₄alkyl)C₁₋₄alkyl;
or both R₅ groups together form a C₃₋₆-spiroalkyl substituted by 0, 1, 2 or 3 substituents selected from halo, cyano, OH, OC₁₋₄alkyl, C₁₋₄alkyl, C₁₋₃haloalkyl, OC₁₋₄alkyl, NH₂, NFIC₁₋₄alkyl, N(C₁₋₄alkyl)C₁₋₄alkyl;
R₆ is selected from H, C₁₋₆haloalkyl, Br, Cl, F, I, ORₐ, NRₐRₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I, and C₁₋₆alkyl;
R₇ is selected from H, C₁₋₆haloalkyl, Br, Cl, F, I, ORₐ, NRₐRₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I and C₁₋₆alkyl;
R₈ is selected from H, halo, C₁₋₄haloalkyl, cyano, nitro, —C(═O)Rₐ, C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(ROS(═O)₂Rₐ, —OC₂₋₆alkylNRₐRₐ, —OC₂₋₆alkylORₐ, —SRₐ, S(═O)Rₐ, S(═O)₂Rₐ, S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, —NRₐRₐ, N(Rₐ)C(═O)Rₐ, N(Rₐ)C(═O)ORₐ, —N(Rₐ)C(═O)NRₐRₐ, N(Rₐ)C(═NRₐ)NRₐRₐ, N(Rₐ)S(═O)₂Rₐ, —N(Rₐ)S(═O)₂NRₐRₐ, —NRₐC₂₋₆alkylORₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from C₁₋₆haloalkyl, OC₁₋₆alkyl, Br, Cl, F, I and C₁₋₆alkyl;
R₉ is selected from H, halo, C₁₋₄haloalkyl, cyano, nitro, —C(═O)Rₐ, —C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(ROS(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, —NRₐRₐ, —N(Rₐ)C(═O)Rₐ, —N(Rₐ)C(═O)ORₐ, —N(Rₐ)C(═O)NRₐRₐ, —N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ, —NRₐC₂₋₆alkylORₐ, C₁₋₆alkyl, phenyl, benzyl, heteroaryl and heterocycle, wherein the C₁₋₆alkyl, phenyl, benzyl, heteroaryl and heterocycle are additionally substituted by 0, 1, 2 or 3 substituents selected from halo, C₁₋₄haloalkyl, cyano, nitro, —C(═O)Rₐ, —C(═O)ORₐ, —C(═O)NRₐRₐ, —C(═NRₐ)NRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, —NRₐRₐ, —N(Rₐ)C(═O)Rₐ, —N(Rₐ)C(═O)ORₐ, —N(Rₐ)C(═O)NRₐRₐ, —N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ, —NRₐC₂₋₆alkylORₐ;
or R₉ is a saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1, or 2 oxo or thioxo groups, wherein the ring is substituted by 0, 1, 2, 3 or 4 substituents selected from halo, C₁₋₄haloalkyl, cyano, nitro, —C(═O)Rₐ, —C(═O)ORₐ, —C(═O)NRₐRₐ, —C(═NRONRₐRₐ, —ORₐ, —OC(═O)Rₐ, —OC(═O)NRₐRₐ, —OC(═O)N(Rₐ)S(═O)₂Rₐ, —OC₂₋₆alkylNRₐRₐ, —OC₂₋₆alkylORₐ, —SRₐ, —S(═O)Rₐ, —S(═O)₂Rₐ, —S(═O)₂NRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, NRₐRₐ, N(Rₐ)C(═O)Rₐ, N(Rₐ)C(═O)ORₐ, —N(Rₐ)C(═O)NRₐRₐ, N(Rₐ)C(═NRₐ)NRₐRₐ, —N(Rₐ)S(═O)₂Rₐ, —N(Rₐ)S(═O)₂NRₐRₐ, —NRₐC₂₋₆alkylNRₐRₐ and —NRₐC₂₋₆alkylORₐ;
R₁₀ is H, C₁₋₃ alkyl, C₁₋₃ haloalkyl, cyano, nitro, CO₂Rₐ, C(═O)NRₐRₐ, —C(═NRONRₐRₐ, —S(═O)₂N(Rₐ)C(═O)Rₐ, —S(═O)₂N(Rₐ)C(═O)ORₐ, —S(═O)₂N(Rₐ)C(═O)NRₐRₐ, S(═O)R_{b}, S(═O)₂R_{b} or S(═O)₂NRₐRₐ;
R₁₁ is H or C₄alkyl;
Rₐ is independently, at each instance, H or R_{b}; and
R_{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, or —N(C₁₋₄alkyl)C₁₋₄alkyl.

12. The method of claim 11, wherein the selective PIK3CD inhibitor is 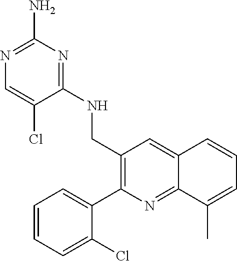
or a pharmaceutically acceptable salt thereof.

13. The method of claim 1, wherein the selective PIK3CD inhibitor is a compound of Formula V: 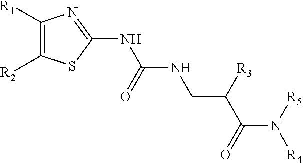
or a pharmaceutically acceptable salt thereof, wherein:
R₁ is C₁₋₃alkyl;
R₂ is phenyl, naphthyl, or biphenylyl, each being optionally substituted by one or more substituents selected from halogen, SO₂C₁₋₃alkyl, acyl and a 5 or 6 membered heteroaryl; or an optionally substituted 5- or 6-membered heteroaryl;
R₃ is H or C₁₋₃alkyl;
R₄ is phenyl, naphthyl or biphenylyl, each being optionally substituted by C₁₋₄alkyl; or an optionally substituted 5- or 6-membered heteroaryl comprising at least one N as heteroatom; provided that R₄ is other than naphthyl when R₂ is phenyl substituted by SO₂C₁₋₃alkyl and optionally halogen; and
R₅ is H or C₁₋₃alkyl.

14. The method of claim 13, wherein the selective PIK3CD inhibitor is: 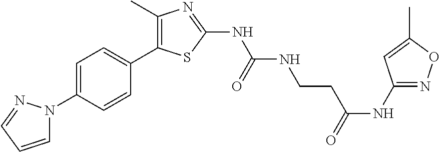
or a pharmaceutically acceptable salt thereof.

15. The method of claim 1 wherein the selective PIK3CD inhibitor is a compound of Formula VI: 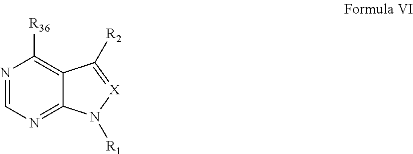
or a pharmaceutically acceptable salt thereof, wherein:
X is N;
R₁ is hydrogen, R₃-substituted or unsubstituted alkyl, unsubstituted cycloalkyl, unsubstituted heterocycloalkyl, or R₃-substituted heteroaryl;
R₂ is R₄-substituted aryl or heteroaryl;
R₃ is halogen, —CN, —OR₅, —S(O)_{N}R₆, —NR₇R₈, —C(O)R₉, —NR₁₀—C(O)R₁₁, —NR₁₂—C(O)—OR₁₃, —C(O)NR₁₄R₁₅, —NR₁₆S(O)₂R₁₇, R₁₉-substituted or unsubstituted alkyl, R₁₉-substituted or unsubstituted heteroalkyl, R₁₉-substituted or unsubstituted cycloalkyl, R₁₉-substituted or unsubstituted heterocycloalkyl, R₁₉-substituted or unsubstituted aryl, or R₁₉-substituted or unsubstituted heteroaryl, wherein n is an integer from 0 to 2;
R₃₆ is —NR₃₇R₃₈;
R₄ is halogen, —OR₂₀, or —NR₂₂R₂₃;
R₅, R₆, R₇, R₈, R₉, R₁₀, R₁₁, R₁₂, R₁₃, R₁₄, R₁₅, R₁₆, and R₁₇ are independently hydrogen, R₃₅-substituted or unsubstituted alkyl, R₃₅-substituted or unsubstituted heteroalkyl, unsubstituted cycloalkyl, R₃₅-substituted or unsubstituted heterocycloalkyl, R₃₅-substituted or unsubstituted aryl, or R₃₅-substituted or unsubstituted heteroaryl;
R₂₀, R₂₂, and R₂₃ are hydrogen;
R₁₉ and R₃₅ are independently hydrogen, halogen, unsubstituted alkyl, unsubstituted heteroalkyl, unsubstituted cycloalkyl, unsubstituted heterocycloalkyl, unsubstituted aryl, or unsubstituted heteroaryl; and
R₃₇ and R₃₈ are hydrogen or C₁-C₆alkyl.

16. The method of claim 1, wherein the selective PIK3CD inhibitor is a compound of Formula VII: 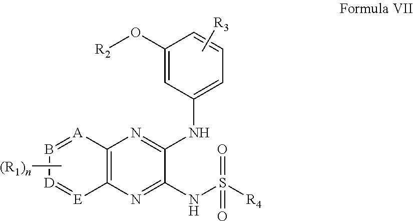
or a pharmaceutically acceptable salt thereof, wherein:
A, B, D and E are independently selected from C and N;
R₁ is selected from H, halogen, nitro, C₁-C₆alkyl, C₂-C₆alkenyl, and C₂-C₆alkynyl;
R₂ is selected from H, C₁-C₆-alkyl, C₂-C₆alkenyl, and C₂-C₆alkynyl;
R₃ is selected from H, halo, C₁-C₆alkyl, C₂-C₆alkenyl, C₂-C₆alkynyl, alkoxy, aryl, and hetero aryl;
R₄ is selected from C₁-C₆alkyl, C₂-C₆alkenyl, C₂-C₆alkynyl, aryl, heteroaryl, C₃-C₈cycloalkyl, heterocycloalkyl, arylC₁-C₆-alkyl, heteroarylC₁-C₆-alkyl, C₃-C₈cycloalkyl C₁-C₆alkyl, heterocycloalkylC₁-C₆alkyl, arylC₂-C₆alkenyl and heteroarylC₂-C₆alkenyl; and
n is an integer selected from 0, 1, 2, 3, and 4.

17. The method of claim 1, wherein the selective PIK3CD inhibitor is a compound of Formula VII or Formula VIII: 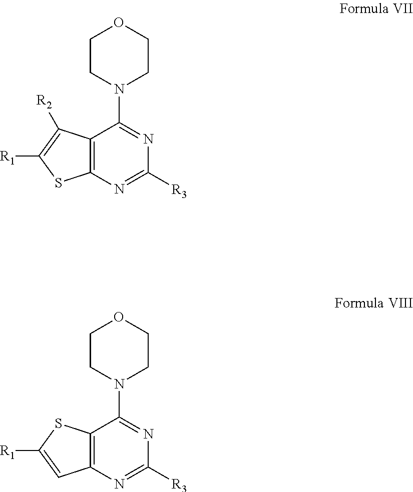
or a pharmaceutically acceptable salt thereof, wherein:
R₁ is —CH₂N(R₄)(R₅);
R₂ is H, halo or C₁-C₆alkyl;
R₃ is an indole group which is unsubstituted or substituted;
R₄ and R₅ form, together with the N atom to which they are attached, a group selected from piperazine, piperidine and pyrrolidine, which group is unsubstituted or substituted by one or more groups selected from C₁-C₆alkyl, —S(O)₂R₁₀, —S(O)₂—
(alk)_{q}-NR₁₁R₁₂, oxo (=0), -alk-OR₁₀, -(alk)_{q}-Het, a heterocyclyl group and —NR₁₃R₁₄; or one of R₄ and R₅ is C₁-C₆=alkyl and the other is a piperazine, piperidine, or pyrrolidine group, which group is unsubstituted or substituted;
R₁₀ is H or C₁-C₆ alkyl which is unsubstituted;
R₁₁ and R₁₂ are each independently selected from H and C₁-C₆alkyl, or R₁₁ and R₁₂ together form, with the N atom to which they are attached, a 5- or 6-membered saturated heterocyclic group;
R₁₃ and R₁₄ are each independently selected from C₁-C₆alkyl, —S(O)₂R₁₀, alk-OR₁₀, -(alk)_{q}-Ph and -(alk)_{q}-Het;
Ph is phenyl;
q is 0 or 1;
Het is a thiazole, imidazole, pyrrole, pyridine or pyrimidine group, which group is unsubstituted or substituted; and
alk is C₁-C₆alkylene.

18-27. (canceled)

28. A method of determining treatment response of a patient with a CNS disorder to a PIK3CD inhibitor, comprising
determining in a biological sample from a patient with a CNS disorder an expression level of a gene that is greater than expression level of the gene determined for a control population lacking the CNS disorder, wherein the gene is PIK3CD or ErbB4 or
determining in the biological sample a level of NRG1-induced phosphatidylinositol-3,4,5-triphosphate ([PI(3,4,5)P3] production or NRG1-induced cell migration that is smaller than the level for the control population lacking the CNS disorder; and
determining that the patient is likely to respond to treatment with an effective amount of a selective PIK3CD inhibitor.

29-36. (canceled)

37. A method of determining risk for a CNS disorder in a human, comprising determining in a nucleic acid sample from a human
a nucleotide base at the polymorphic site rs6540991 (position 201 of SEQ ID NO: 3) is a thymine (T),
a nucleotide base at the polymorphic site rs9430220 (position 401 of SEQ ID NO: 10) is a thymine (T);
a nucleotide base at the polymorphic site rs12567553 (position 501 of SEQ ID NO: 4) is an adenine(A),
a nucleotide base at the polymorphic site rs9694151 (position 900 of SEQ ID NO: 9) is an adenine(A);
a nucleotide base at the polymorphic site rs6660363 (position 1437 of SEQ ID NO: 6) is an adenine(A),
a nucleotide base at the polymorphic site rs4601595 (position 301 of SEQ ID NO: 7) is a guanine (G),
a nucleotide base at the polymorphic site rs12037599 (position 401 of SEQ ID NO: 12) is a guanine (G),
a nucleotide base at the polymorphic site rs1135427, (position 401 of SEQ ID NO: 13) is a thymine (T),
a nucleotide base at the polymorphic site rs1141402, (position 201 of SEQ ID NO: 14) is a guanine (G), or
a genotype at the polymorphic site rs11589267 (position 401 of SEQ ID NO: 11) is TC; or
determining in a nucleic acid sample from a human the genotype of each polymorphic site in a pair of polymorphic sites, wherein the determined genotypes in the pair of polymorphic site is
AA at the polymorphic site rs707284 (position 559 of SEQ ID NO: 17) and TT at the polymorphic site rs4601595 (position 301 of SEQ ID NO: 7),
G carrier at the polymorphic site rs839539 (position 451 of SEQ ID NO: 18) and A carrier at the polymorphic site rs11801864 (position 501 of SEQ ID NO: 8);
T carrier at the polymorphic site rs1098059 (position 1773 of SEQ ID NO: 19) and A carrier at the polymorphic site rs11801864 (position 501 of SEQ ID NO: 8),
AA at the polymorphic site rs7598440 (position 301 of SEQ ID NO: 15) and GG at the polymorphic site rs4601595 (position 301 of SEQ ID NO: 7),
G carrier at the polymorphic site rs839539 (position 451 of SEQ ID NO: 18) and A carrier at the polymorphic site rs7518793 (position 976 of SEQ ID NO: 2),
TT at the polymorphic site rs839541 (position 401 of SEQ ID NO: 16) and GG at the polymorphic site rs12037599 (position 401 of SEQ ID NO: 17),
T carrier at the polymorphic site rs1098059 (position 1773 of SEQ ID NO: 19) and G carrier at the polymorphic site rs12567553 (position 501 of SEQ ID NO: 4),
C carrier at the polymorphic site rs62185768 (position 251 of SEQ ID NO: 21) and CC at the polymorphic site rs9430635 (position 251 of SEQ ID NO: 5), or
C carrier at the polymorphic site rs62185768 (position 251 of SEQ ID NO: 21) and AA at the polymorphic site rs6660363 (position 1437 of SEQ ID NO: 6); and
determining that the human having a T at rs6540991 or rs9430220 has an increased risk for a CNS disorder;
determining that the human determined to have
AA at rs707284 and TT at rs4601595,
G carrier at rs839539 and A carrier at rs11801864,
T carrier at rs1098059 and A carrier at rs11801864,
AA at rs7598440 and GG at rs4601595,
G carrier at rs839539 and A carrier at rs7518793,
TT at rs839541 and GG at rs12037599,
T carrier at t rs1098059 and G carrier at rs12567553,
C carrier at rs62185768 and CC at rs9430635, or
C carrier at rs62185768 and AA at rs6660363 has an increased risk for a CNS disorder;
determining that the human having an A at rs12567553 or rs9694151 has an increased risk for a CNS disorder if the human is an African-American; or
determining that the human having an A at rs6660363, a G at rs4601595, a G at rs12037599, a T at rs1135427; a G at rs1141402, or a TC at rs11589267 has an increased risk for a CNS disorder if the human is a Caucasian.

38. A method of determining treatment response of a patient with a CNS disorder to a PIK3CD inhibitor, comprising
determining in a nucleic acid sample from a patient with a CNS disorder
a nucleotide base at the polymorphic site rs6540991 (position 201 of SEQ ID NO: 3) is a thymine (T),
a nucleotide base at the polymorphic site rs9430220 (position 401 of SEQ ID NO: 10) is a thymine (T);
a nucleotide base at the polymorphic site rs12567553 (position 501 of SEQ ID NO: 4) is an adenine(A),
a nucleotide base at the polymorphic site rs9694151 (position 900 of SEQ ID NO: 9) is an adenine(A);
a nucleotide base at the polymorphic site rs6660363 (position 1437 of SEQ ID NO: 6) is an adenine(A),
a nucleotide base at the polymorphic site rs4601595 (position 301 of SEQ ID NO: 7) is a guanine (G),
a nucleotide base at the polymorphic site rs12037599 (position 401 of SEQ ID NO: 12) is a guanine (G),
a nucleotide base at the polymorphic site rs1135427, (position 401 of SEQ ID NO: 13) is a thymine (T),
a nucleotide base at the polymorphic site rs1141402, (position 201 of SEQ ID NO: 14) is a guanine (G), or
a genotype at the polymorphic site rs11589267 (position 401 of SEQ ID NO: 11) is TC; or
determining in a nucleic acid sample from a patient with a CNS disorder the genotype of each polymorphic site in a pair of polymorphic sites, wherein the determined genotype s in the pair of polymorphic site is
AA at the polymorphic site rs707284 (position 559 of SEQ ID NO: 17) and TT at the polymorphic site rs4601595 (position 301 of SEQ ID NO: 7),
G carrier at the polymorphic site rs839539 (position 451 of SEQ ID NO: 18) and A carrier at the polymorphic site rs11801864 (position 501 of SEQ ID NO: 8);
T carrier at the polymorphic site rs1098059 (position 1773 of SEQ ID NO: 19) and A carrier at the polymorphic site rs11801864 (position 501 of SEQ ID NO: 8),
AA at the polymorphic site rs7598440 (position 301 of SEQ ID NO: 15) and GG at the polymorphic site rs4601595 (position 301 of SEQ ID NO: 7),
G carrier at the polymorphic site rs839539 (position 451 of SEQ ID NO: 18) and A carrier at the polymorphic site rs7518793 (position 976 of SEQ ID NO: 2),
TT at the polymorphic site rs839541 (position 401 of SEQ ID NO: 16) and GG at the polymorphic site rs12037599 (position 401 of SEQ ID NO: 17),
T carrier at the polymorphic site rs1098059 (position 1773 of SEQ ID NO: 19) and G carrier at the polymorphic site rs12567553 (position 501 of SEQ ID NO: 4),
C carrier at the polymorphic site rs62185768 (position 251 of SEQ ID NO: 21) and CC at the polymorphic site rs9430635 (position 251 of SEQ ID NO: 5), or
C carrier at the polymorphic site rs62185768 (position 251 of SEQ ID NO: 21) and AA at the polymorphic site rs6660363 (position 1437 of SEQ ID NO: 6); and
determining that the patient determined to have T at rs6540991 or rs9430220 is likely to respond to treatment with an effective amount of a selective PIK3CD inhibitor,
determining that the patient determined to have
AA at t rs707284 and TT at rs4601595,
G carrier at rs839539 and A carrier at rs11801864;
T carrier at rs1098059 and A carrier at rs11801864,
AA at rs7598440 and GG at rs4601595,
G carrier at rs839539 and A carrier at rs7518793,
TT at rs839541 and GG at rs12037599,
T carrier at rs1098059 and G carrier at rs12567553,
C carrier at rs62185768 and CC at rs9430635, or
C carrier at rs62185768 and AA at rs6660363 is likely to respond to treatment with an effective amount of a selective PIK3CD inhibitor,
determining that the patient having A at rs12567553 or rs9694151 is likely to respond to treatment with an effective amount of a selective PIK3CD inhibitor if the patient is African-American, or
determining that the patient having A at rs6660363, G at rs4601595, G at rs12037599, T at rs1135427, G at rs1141402, or TC at rs11589267 is likely to respond to treatment with an effective amount of a selective PIK3CD inhibitor if the patient is Caucasian.

39. The method of claim 5, wherein in the structure of formula I, A is ring of the formula 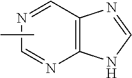
and Y is NH.
